(19) United States
(12) Patent Application Publication
(10) Pub. No.: US 2004/0002524 A1
(43) Pub. Date: Jan. 1, 2004

(54) Benzimidazole compounds and their use as estrogen agonists/antagonists

(76) Inventors: Richard Chesworth, Mystic, CT (US); Laura D. Gegnas, Killingworth, CT (US)

Correspondence Address: PFIZER INC., PATENT DEPARTMENT, MS8260-1611, EASTERN POINT ROAD, GROTON, CT, 06340, US

(21) Appl. No.: 10/460,565

(22) Filed: Jun. 11, 2003

Related U.S. Application Data
(60) Provisional application No. 60/391,337, filed on Jun. 24, 2002.

(51) Int. Cl. A61K 31/433; A61K 31/425; A61K 31/42; A61K 31/4184; C07D 417/02; C07D 413/02; C 07D 4 3/02

(52) U.S. Cl. 514/361; 514/372; 514/378; 514/394; 548/127; 548/206; 548/240; 548/304.7; 548/306.1

(57) ABSTRACT

This invention relates to compounds, in particular benzimidazoles, that are useful as estrogen agonists and/or antagonists and pharmaceutical uses thereof. The present invention also relates to benzimidazoles that are selective for the ERβ receptor and pharmaceutical uses thereof.

### FIELD OF THE INVENTION

This invention relates to compounds, in particular benzimidazoles, that are useful as estrogen agonists/antagonists and pharmaceutical uses thereof. The present invention also relates to benzimidazoles that are selective for the ERβ receptor and pharmaceutical uses thereof.

### BACKGROUND OF THE INVENTION

As a mediator of the actions of estrogenic hormones, the estrogen receptor (ER) plays a central role in regulating a diverse array of normal physiological processes involved in the development and function of the reproductive system, as well as many other aspects of health, such as bone density, cardiovascular health, etc.

It is known that compounds that bind to a ER are potentially useful in the treatment of a wide range of disease states. These include estrogen agonists for treatment of disease linked to estrogen deficiency, such as osteoporosis, cardiovascular and neurodegenerative diseases in post menopausal women; and estrogen antagonists for treatment of breast and uterine cancer. Furthermore, it is known that certain ligands, such as tamoxifen display mixed agonist/antagonist action; that is they are either estrogen agonists, estrogen antagonists or a partial estrogen antagonist when binding to the estrogen receptors of different tissues.

Estrogen is the agent of choice in preventing osteoporosis or post menopausal bone loss in women, it is the only treatment that unequivocally reduces fractures. However, estrogen stimulates the uterus and is associated with an increased risk of endometrial cancer. Although the risk of endometrial cancer is thought to be reduced by concurrent use of a progestin, there remains concern about possible increased risk of breast cancer with the use of an estrogen.

It would be desirable to be able to produce ligands which are recognizable by and able to bind to the estrogen receptor. Further, it would be desirable to produce ligands having estrogen-like function, but which are devoid of unwanted side-effects of estrogenic compounds. For example, osteoporosis is greatly ameliorated by the use of fully active estrogens; however, due to the recognized risk of uterine cancer in patients treated chronically with active estrogens, it is not clinically advisable to treat osteoporosis with fully active estrogens for prolonged periods.

Until recently, it has been assumed that estrogen binds to a single estrogen receptor (ER) in cells, causing conformational changes that result in release from heat shock proteins and binding of the receptor as a dimer to the so-called estrogen response element in the promoter region of a variety of genes.

Recently, a second estrogen receptor, ERβ, has been identified and cloned (Katzenellenbogen and Korach Endocrinology 138, 861-2 (1997). ERβ, and the classical ER, renamed ERα, have significantly different amino acid sequences in the ligand binding domain and carboxy-terminal transactivation domains (˜56% amino acid identity) and only 20% homology in their amino-terminal transactivation domain. This suggests that some ligands may have higher affinity to one receptor over the other. Further, ligand-dependent conformational changes of the two receptors, and interaction with co-factors, will result in very different biological actions of a single ligand. In other words, a ligand that acts as an agonist on ERα may very well serve as an antagonist on ERβ. An example of such behavior has been described by Paech et al. (Science 277,1508-1510, 1997).

In addition, it has been found that there are differences in the proportion of expression of ERβ and ERα in different organs. For example, organs in which there is a high proportion of ERα receptors include the uterus and the hypothalmus. ERβ is highly expressed in large amounts in ovaries and bone.

With the recent identification of ERβ, and the recognition that ERβ and ERα have different tissue distributions, ER-selective modulators would possess significant clinical utility. Further, ER-selective modulators that have the capacity to selectively bind or activate the ER subtypes, ERβ and ERα, would be useful in elucidating the biology of the two receptors and will assist in the development of estrogen pharmaceuticals with improved tissue selectivity.

### SUMMARY OF THE INVENTION

In a first aspect, the invention relates to a compound of formula (I):

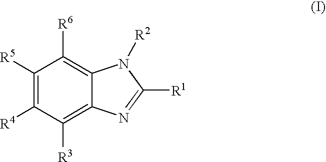

or the pharmaceutically acceptable salts thereof; wherein:

R¹ and R² are each independently selected from the group consisting of (C₁-C₆)alkyl; phenyl; (C₂-C₆)heteroaryl; (C₃-C₈)cycloalkyl; and (C₄-C₈)cycloalkenyl;

wherein the (C₁-C₆)alkyl; phenyl; (C₂-C₆)heteroaryl; (C₃-C₈)cycloalkyl; or (C₄-C₈)cycloalkenyl groups of R¹ or R² are optionally substituted by from 1 to 3 substituents independently selected from the group consisting of:

halogen; (C₁-C₆)alkyl; (C₃-C₈)cycloalkyl; (C₄-C₈)cycloalkenyl; (C₁-C₆)alkoxy; hydroxy; R¹² CO₂, R¹²R¹³NCO, R¹²R¹³N; (C₁-C₆)alkylcarbonyl, —CHO, cyano, thio; (C₁-C₆)alkylthio; (C₁-C₆)alkylsulfonyl; (C₁-C₆)alkylsulfinyl; hydroxy(C₁-C₆)alkyl; (C₁-C₆)alkoxycarbonylamino; (C₁-C₆)alkylcarbonylamino; (C₁-C₆)alkenylcarbonylamino; (C₁-C₆)alkoxycarbonyloxy; R¹² R¹³N(C₁-C₆); R¹²R¹³N(C₁-C₆)alkoxy; R¹²R¹³N(C₁-C₆alkyl)S; N-morpholino(CH₂)ₙO; or —R¹²R¹³N(CH₂)ₙS(O)ₓ; wherein the (C₁-C₆)alkyl; (C₃-C₈)cycloalkyl; (C₄-C₈)cycloalkenyl; (C₁-C₆)alkoxy; (C₁-C₆)alkylcarbonyl; (C₁-C₆)alkylthio; (C₁-C₆)alkylsulfonyl; (C₁-C₆)alkylsulfinyl; (C₁-C₆)alkoxycarbonylamino; (C₁-C₆)alkylcarbonylamino; (C₁-C₆)alkenylcarbonylamino; or (C₁-C₆)alkoxycarbonyloxy groups are each optionally further substituted by from 1 to 3 substituents independently selected from the group consisting of:

halogen, (C₁-C₆)alkyl; (C₃-C₈)cycloalkyl; (C₄-C₈)cycloalkenyl; (C₁-C₆)alkoxy, hydroxy, R¹² CO₂, R¹²R¹³NCO, R¹²R¹³N;(C₁-C₆)alkylcarbonyl, —CHO, cyano, thio; R¹²SO₂(C₁-C₆)alkyl; R¹² CO₂(C₁-C₆)alkyl; R¹²R¹³NCO(C₁-C₆)alkyl; R¹² CO(C₁-C₆)alkyl; R¹² SO₂(C₁-C₆)alkoxy; R¹² CO₂(C₁-C₆)alkoxy; R¹²R¹³NCO(C₁-C₆)alkoxy; R¹²CO(C₁-C₆)alkoxy; R¹²R¹³ N SO₂(C₁-C₆)alkyl; and R¹²R¹³N SO₂(C₁-C₆) alkoxy; wherein:

R¹² and R¹³ are each independently selected from the group consisting of hydrogen; (C₁-C₇)alkyl; (C₃-C₈)cycloalkyl; (C₄-C₈)cycloalkenyl;(C₆-C₁₀) aryl; (C₂-C₁₀)alkenyl, (C₂-C₁₀)alkynyl; (C₂-C₄)heteroaryl; (C₁-C₆)alkylaryl; (C₁-C₆) alkyl(C₂-C₆)heteroaryl; (C₂-C₆)alkoxyaryl; (C₂-C₆) alkoxy(C₂-C₆)heteroaryl; or R¹² and R¹³ taken together form a three to eight membered heterocyclic ring having 1 to 3 heteroatoms; n is from 0 to 5; and x is 1 or 2;

or R¹ and R² are each independently a group of the formula:

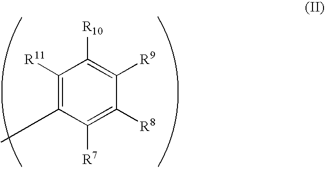

wherein R⁷, R⁸, R¹⁰ and R¹¹ are each independently hydrogen; hydroxy; (C₁-C₆) alkyl; (C₁-C₆)alkoxy; or halogen;

R⁹ is hydroxy; (C₁-C₆) alkoxy; (C₁-C₆)alkoxycarbonyloxy; (C₁-C₆)alkylcarbonyloxy; (C₃-C₈)cycloalkoxy; (C₄-C₈)cycloalkenyloxy; or (C₆-C₁₂) aryloxy; and

R³, R⁴, R⁵ and R⁶ are each independently hydrogen, hydroxy; (C₁-C₆)alkyl; (C₁-C₆)alkoxy; or halogen

with the proviso that at least one of R¹ or R² must be the group of formula (II)

Throughout the application, where publications (including, but not limited to, U.S. patents) are referenced, the disclosures of these publications in their entireties are hereby incorporated by reference.

In the specification and claims that follow, reference will be made to a number of terms which shall be defined to have the following meaning.

The singular forms “a”, “an” and “the” include plural referents unless the context clearly dictates otherwise.

“Optional” or “optionally” means that the subsequently described event or circumstance may or may not occur, and that the description includes instances where the event or circumstances occurs and instances where it does not.

The term “alkyl” refers to straight or branched, monovalent, saturated aliphatic chains having the number of carbon atoms designated and includes, but is not limited to methyl, ethyl, propyl, isopropyl, butyl, isobutyl, t-butyl, pentyl, isopentyl, and hexyl.

The term “alkenyl” refers to straight or branched chain hydrocarbon groups of 2 to 10 carbon atoms having at least one double bond.

The term “alkynyl” refers to straight of branched chain hydrocarbon groups of 2 to 10 carbon atoms having at least one triple bond.

The term “aryl” refers to monocylic and polycyclic aromatic groups, or fused ring systems having at least one aromatic ring, having from 5 to 14 backbone atoms. Examples of aryl groups include, without limitation, phenyl, naphthyl, dihydronaphthyl, tetrahydronapthyl, and the like.

“Cycloalkyl” groups means a cyclic hydrocarbon. Examples of cycloalkyl groups include cyclopropyl, cyclobutyl, cyclopentyl, cyclohexyl and cycloheptyl. Preferred cycloalkyl groups are (C₃-C₈)cycloalkyl. It is also possible for the cycloalkyl group to have one or more double bonds, but is not aromatic. Cycloalkyl having at least one double bond are herein referred to as “cycloalkenyl” groups. Examples of cycloalkyl groups having at least one double bond include cyclopentenyl, cyclohexenyl, cyclohexadienyl, cyclobutadienyl, and the like.

“Heteroaryl” means an aryl ring containing one or more heteroatoms. If the heteroaryl group contains more than one heteroatom, the heteroatom may be the same or different. Examples of heteroaryl groups include pyridyl, pyrimidinyl, imidazolyl, thienyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, benzo[b]thienyl, isooxazolyl, isothiazolyl and thiodiazolyl.

The term “heteroatom” includes oxygen, nitrogen and sulphur. A cycloalkyl group having at least one heteroatom is a “heterocycle”.

The term “substituted” means that a hydrogen atom on a molecule has been replaced with a different atom or molecule. The atom or molecule replacing the atom is denoted as a “substituent.” The term “substituted” specifically envisions and allows for substitutions that are common in the art. However, it is generally understood by those skilled in the art that the substituents should be selected so as to not adversely affect the pharmacological characteristics or adversely interfere with the use of the medicament. Suitable substituents include halogen; (C₁-C₆)alkyl; (C₃-C₈)cycloalkyl; (C₄-C₈)cycloalkenyl; (C₁-C₆)alkoxy; hydroxy; R¹² CO₂, R¹²R¹³NCO, R¹²R¹³N; (C₁-C₆) alkylcarbonyl, CHO, cyano, thio; (C₁-C₆)alkylthio; (C₁-C₆)alkylsulfonyl; (C₁-C₆)alkylsulfinyl;CH₂OH; (C₁-C₆)alkoxycarbonylamino; (C₁-C₆)alkylcarbonylamino; (C₁-C₆)alkenylcarbonylamino; (C₁-C₆)alkoxycarbonyloxy; R¹² R¹³ N(C₁-C₆); R¹²R¹³N(C₁-C₆) O; R¹²R¹³N(C₁-C₆) S; N-morpholino(CH₂)ₙO; and —R¹²R¹³N(CH₂)S(O)ₓ. R¹² and R¹³ are as defined in Formula (I).

When the term “alkyl” is used to suffix another group, such as in “arylalkyl”, “heterocycloalkyl”, “cycloalkylalkyl,” or “heteroarylalkyl” the term defines with more specificity at least one of the groups that a substituted alkyl will contain. In other words, in these instances the specifically named groups are bonded directly through a substituted or unsubstituted alkyl chain, as defined.

An “estrogen agonist/antagonist” is a compound that acts as an agonist at some receptors and an antagonist at other receptor. Estrogen agonists/antagonists are also known as selective estrogen receptor modulators (SERMs).

The term “prodrug” refers to compounds that are drug precursors which, following administration, release the drug in vivo via some chemical or physiological process (e.g. a prodrug on being brought to the physiological pH or through enzyme action is converted to the desired drug form).

The term “Estrogen Receptor” as used herein refers to ERβ and/or the ERα.

“Estrogen Receptor Modulators” are compounds that bind to the ERPβ and/or the ERα receptors and function as estrogen agonists/estrogen antagonists.

An “ERβ selective estrogen receptor modulator” is a compound that selectively binds to the ERβ receptor. By “selective” it is meant that the compound exhibits at least 5 times the binding affinity for the ERβ than the ERα receptor as indicated by IC₅₀ in a competitive binding assay. By “more selective” it is meant that the compound exhibits at least 50 times the binding affinity for the ERβ than the ERα receptor as indicated by IC₅₀ in a competitive binding assay. By “selectively antagonizing or agonizing” as used in the present specification, it is meant that the compound is selective or more selective for the ERβ receptor and exhibits agonist and/or antagonist activity.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The phrase “pharmaceutically acceptable” indicates that the substance or composition must be compatible chemically and/or toxicologically, with the other ingredients comprising a formulation, and/or the mammal being treated therewith.

The expression “pharmaceutically-acceptable salt” refers to nontoxic anionic salts containing anions such as (but not limited to) chloride, bromide, iodide, sulfate, bisulfate, phosphate, acetate, maleate, fumarate, oxalate, lactate, tartrate, citrate, gluconate, methanesulfonate and 4-toluene-sulfonate. Where more than one basic moiety exists the expression includes multiple salts (e.g., di-salt). The expression also refers to nontoxic cationic salts such as (but not limited to) sodium, potassium, calcium, magnesium, ammonium or protonated benzathine (N,N′-dibenzylethylenediamine), choline, ethanolamine, diethanolamine, ethylenediamine, meglamine (N-methyl-glucamine), benethamine (N-benzylphenethylamine), piperazine or tromethamine (2-amino-2-hydroxymethyl-1,3-propanediol).

The term “female sexual dysfunction” as used herein includes hypoactive sexual desire disorder, sexual anhedonia and dyspareunia. Hypoactive sexual desire disorder is a disorder in which sexual fantasies and desire for sexual activity are persistently or recurrently diminished or absent, causing marked distress or interpersonal difficulties. Hypoactive sexual desire disorder may be lifelong or acquired, generalized (global) or situational (partner-specific). Sexual desire is a complex psychosomatic process based on brain activity (the “generator” or “motor” running in a rheostatic cyclic fashion), a poorly defined hormonal milieu, and cognitive scripting that includes sexual aspiration and motivation. Desynchronization of these components results in hypoactive sexual desire disorder.

Sexual anhedonia (decreased or absent pleasure in sexual activity) is not an official diagnosis. It is almost always classified under hypoactive sexual desire disorder, because loss of pleasure almost always results in loss of desire (although loss of desire may occur first). The cause is likely to be depression or drugs if anhedonia is acquired and global (with all partners in all situations); interpersonal factors if anhedonia is confined to one partner or one situation; or repressive factors (eg. guilt, shame) due to family dysfunction or childhood trauma if anhedonia is lifelong. Sexual aversion is the probable diagnosis in lifelong cases.

Dyspareunia is painful coitus or attempted coitus. Dyspareunia is usually introital but may also occur before, during, or after intercourse. Causes include menopausal involution with dryness and thinning of the mucosa. Pain during or after coitus is the chief complaint.

A chemist of ordinary skill will recognize that certain compounds of this invention will contain atoms which may be in a particular optical or geometric configuration, including but not limited to stereoisomers, diastereomers and mixtures thereof. All such isomers are included in this invention in reference to compounds of formula (I) Similarly, the chemist will recognize that various pharmaceutically acceptable esters and salts may be prepared from certain compounds of this invention. All such esters and salts are included in this invention in reference to compounds of formula (I).

In a further embodiment of the first aspect, the invention relates to a compound of formula (I), wherein R¹ is phenyl or (C₂-C₆) heteroaryl. In yet another embodiment of the first aspect of the invention, the (C₂-C₆) heteroaryl is thienyl; furyl; pyrrolyl; isoxazolyl; isothiazolyl or thiodiazolyl.

In a further embodiment of the first aspect, the invention relates to a compound according to formula (I), wherein R¹ is phenyl optionally substituted by R¹²CO₂ or R¹²R¹³NC(O).

In a further embodiment of the first aspect, the invention relates to a compound according to formula(I), wherein R² is a group of formula (II) In an even further embodiment of the first aspect, the invention relates to a compound according to formula (I) wherein R² is a group of formula (II),wherein R⁷, R⁸, R¹⁰ and R¹¹ are hydrogen arid R⁹ is hydroxy or (C₁-C₆)alkoxy.

In a further embodiment of the first aspect, the invention relates to a compound according to formula (I), wherein R³, R⁴, R⁵ and R⁶ are hydrogen.

In a further embodiment of the first aspect, the invention relates to a compound according to formula (I) wherein R¹ is phenyl or (C₂-C₆)heteroaryl; R²is a group of formula (II); and R³, R⁴, R⁵ and R⁶ are hydrogen. In a further refinement of this embodiment, the (C₂-C₆)heteroaryl is thienyl; furyl; pyrrolyl; isoxazolyl; isothiazoyl or thiodiazolyl, R⁷, R⁸, R¹⁰ and R¹¹ are hydrogen; and R⁹ is hydroxy or (C₁-C₆)alkoxy.

In a further embodiment, the invention relates to a compound of Formula (I), wherein R¹ is phenyl optionally substituted by R ²CO₂ or R¹²R¹³NC(O); R² is a group of formula (II); and R³, R⁴, R⁵ and R⁶ are hydrogen.

In a further embodiment, the invention relates to a compound of formula (I) or the pharmaceutically accepted salts thereof, wherein the compound of formula (I) is selected from the group consisting of:

(±)-4-(2-sec-butyl-benzoimidazol-1-yl)-phenol;

4-(2-y-clopropyl-benzoimidazol-1-yl)-phenol 4-[2-(4-iodo-phenyl)-benzoimidazol-1-yl]-phenol;

4-(2-thiophen-3-yl-benzoimidazol-1-yl-phenol;

4-(2-thiophen-2-yl-benzoimidazol-1-yl)phenol;

4-[2-(1-methyl-1H-pyrrol-2-yl)-benzoimidazol-1-yl]-phenol;

4-[2-(3,5-dimethyl-isoxazol-4-yl)-benzoimidazol-1-yl)-phenol;

4-[2-(3-bromo-thiophen-2-yl)-benzoimidazol-1-yl]-phenol;

4-(2-isothiazol-4-yl-benzoimidazol-1-yl)-phenol;

4-[2-(4-methyl-isothiazol-5-yl)-benzoimidazol-1-yl]-phenol;

4-[2-(4-methyl-[1,2,3]thiadiazol-5-yl)-benzoimidazol-1-yl]-phenol;

4-[2-(3-chloro-thiophen-2-yl)-benzoimidazol-1-yl]-phenol;

4-[2-(1-ethyl-1H-pyrrol-2-yl)-benzoimidazol-1-yl]-phenol;

4-(2-furan-3-yl-benzoimidazol-1-yl-phenol;

4-[2-(3-methyl-furan-2-yl)-benzoimidazol-1-yl]-phenol;

4-(2-furan-2-yl-benzoimidazol-1-yl)-phenol;

4-[2-(3-ethyl-isoxazol-4-yl)-benzoimidazol-1-yl-phenol;

4-[2-(3-cyclopropyl-isoxazol4-yl)-benzoimidazol-1-yl]-phenol;

4-[2-(3-ethyl-5-methyl-isoxazol-4-yl)-benzoimidazol-1-yl]-phenol;

4-[2-(5-methyl-isoxazol-4-yl)-benzoimidazol-1-yl]-phenol;

4-[2-(3-methyl-isoxazol-4-yl]-phenol;

4-[2-(2-methyl-thiophen-3-yl)-benzoimidazol-1-yl]-phenol;

4-[2-(2-methyl-furan-3-yl)-benzoimidazol-1-yl]-phenol;

4-[2-(2,5-dimethyl-furan-3-yl)benzoimidazol-1-yl]-phenol;

4-[2-(2,5-dimethyl-furan-3-yl)benzoimidazol-1-yl]-phenol;

4-[2-(1-propyl-1H-pyrrol-2-yl)-benzoimidzol-1-yl]-phenol;

4-[2-(1-isopropyl-1H-pyrrol-2-yl)-benzoimidazol-1-yl]-phenol;

3-methyl-4-[2-(1-methyl-1H-pyrrol-2-yl)-benzoimidazol-1-yl]-phenol;

4-[2-(3,5-dimethyl-isoxazol-4-yl)-benzoimidazol-1-yl]-3-methyl-phenol;

4-[2-(3-methyl-thiophen-2-yl)-benzoimidazol-1-yl]-phenol;

4-(2-isothiazol-5-yl-benzoimidazol-1-yl)-phenol;

4-[i -(4-Hydroxy-phenyl)-1H-benzoimidazol-2-yl]-benzoic acid methyl ester;

4-[1-(4-Hydroxy-phenyl)-1H-benzoimidazol-2-yl]-benzoic acid ethyl ester;

4-[1-(4-Hydroxy-phenyl)-1H-benzoimidazol-2-yl]-benzoic acid isopropyl ester;

4-[1-(4-Hydroxy-phenyl)-1H-benzoimidazol-2-yl]-N-isopropyl-benzamide;

4-[1-(4-Hydroxy-phenyl)-1H-benzoimidazol-2-yl]-N-(1-phenyl-ethyl)-benzamide;

4-[1-(4-Hydroxy-phenyl)-1H-benzoimidazol-2-yl]-N-(1-phenyl-ethyl)-benzamide; and

4-[1-(4-Hydroxy-phenyl)-1H-benzoimidazol-2-yl]-N-thiophen-2-ylmethyl-benzamide.

These compounds are effective as estrogen receptor modulators.

In a second aspect, the invention relates to a pharmaceutical composition for antagonizing or agonizing an estrogen receptor in a mammal comprising an estrogen receptor antagonizing or agonizing effective amount of a compound of formula (I) according to claim 1, or a pharmaceutically accepted salt thereof, and a pharmaceutically acceptable carrier.

In a third aspect, the invention relates to a pharmaceutical composition for selectively antagonizing or agonizing an ERβ estrogen receptor in a mammal comprising an ERβ estrogen receptor antagonizing or agonizing effective amount of a compound of formula (I) or the pharmaceutically acceptable salts thereof and a pharmaceutically acceptable carrier.

In a fourth aspect, the invention relates to a pharmaceutical composition comprising an agent selected from the group consisting of an anabolic agent; a growth hormone; a growth hormone secretagogue; a prostaglandin agonist/antagonist; a parathyroid hormone; sodium fluoride; and a mixture thereof; the pharmaceutical composition further comprising a compound of formula (I).

In a fifth aspect, the invention relates to a method of treating a condition which presents with low bone mass in a mammal comprising administering to the mammal a compound of formula (I), a prodrug thereof or a pharmaceutically acceptable salt, or a stereoisomeric mixture of said compound, salt or prodrug. In one embodiment of the fifth aspect, the condition is osteoporosis.

In a sixth aspect, the invention relates to a kit comprising: a) an amount of a compound of Formula (I) as defined in claim 1; b) an amount of a second compound comprising an anabolic agent; a growth hormone; a growth hormone secretagogue; a prostaglandin agonist/antagonist; a parathyroid hormone; sodium fluoride; or a mixture thereof; and c) a container.

In a seventh aspect, the invention relates to a method of treating a disease mediated by the estrogen receptor in a mammal, comprising administering to the mammal a therapeutically effective amount of a compound of formula (I) according to claim 1 in a pharmaceutically effective carrier.

In one embodiment of the seventh aspect, the disease is selected from the group consisting of perimenopausal or postmenopausal syndrome, osteoporosis, atrophy of skin or vagina, elevated serum cholesterol levels, cardiovascular disease, Alzheimer's disease, estrogen dependent cancers, including breast or uterine cancer, a prostatic disease, benign prostatic hyperplasia, prostate cancer, obesity, endometriosis, bone loss, uterine fibrosis, aortal smooth muscle cell proliferation, acne, hirsutism, dysfunctional uterine bleeding, dysmenorrehea, male infertility, male erectile dysfunction (MED), psychological and behavioral symptoms during menstruation, ulcerative mucositis, uterine fibroid disease, restenosis, atherosclerosis, musculoaponeurotic fibromatosis, alopecia, autoimmune disease, cartilage degeneration, delayed puberty, demyelinating disease, dysmyelinating disease, hypoglycemia, lupus erythematosus, myocardial infection, ischemia, thromboembolic disorder, obsessive compulsive disorder, ovarian dysgenesis, post menopausal central nervous system (CNS) disorder, pulmonary hypertension, reperfusion damage, resistant neoplasm, rheumatoid arthritis, seborrhea, sexual precocity, thyroiditis, Turner's syndrome, and hyperlipidemia and female sexual dysfunction.

In an eight aspect, the invention relates to a method for selectively antagonizing or agonizing an ERβ estrogen receptor in a mammal comprising an ERβ estrogen receptor antagonizing or agonizing effective amount of a compound of formula (I).

The present invention relates to compounds that have activity as estrogen receptor modulators, as well as pharmaceutical compositions containing one or more of such compounds and methods of use related to the same. As estrogen receptor modulators, the compounds of this invention have utility in the treatment of a wide range of estrogen-related conditions. Thus, the compounds of this invention may be administered as a therapeutic and/or prophylactic agent. Certain compounds within the class of estrogen receptor modulators as described herein were found to be selective for the ERβ receptor, and certain compounds within the class of ERβ selective compounds were found to be more selective for the ERβ receptor.

It is preferable that the compounds of the invention have an IC₅₀ with respect to ERβ and/or ERα of no more than 500 nanomolar.

As an even further aspect of the invention, it was unexpectedly found that some compounds of formula (I) were selective for the ERβ receptor. In order to determine whether a compound is selective or more selective for the ERβ receptor, an assay may be performed as described in the present specification in the section entitled “assay for estrogen receptor binding activity”. Preferred compounds compounds of formula (I) that are selective for the ERβ receptor are compounds of formula (I) in which R₁ represents a five membered heteroaryl ring having up to 3 heteroatoms independently selected from N, O and S. Suitable five membered heteroaryl rings include, but are not limited to, thienyl ; furyl; pyrrolyl; isoxazolyl; and thiodazolyl groups; preferably thienyl, furyl, pyrrolyl and isoxazolyl groups . As described, the five membered heteroaryl ring selected as R₁ may optionally be substituted. Examples of the substituted R₁ groups include, but are not limited to, 1-methyl-1H-pyrrol-2-yl; 3,5-dimethyl-isoxazol-4-yl; 3-bromo-thiopen-2-yl; and 1-ethyl-1H-pyrrol-2-yl. In one embodiment of the invention, R₁ represents a five membered heteroaryl ring having up to 3 heteroatoms independently selected from N, O, and S; and R₉ represents OH.

Compounds of formula (I) that are selective for the ERβ receptor include, but are not limited to:

4-(2-thiophen-3-yl-benzoimidazol-1-yl-phenol;

4-(2-thiophen-2-yl-benzoimidazol-1-yl)phenol; 4-[2-(1-methyl-1H-pyrrol-2-yl)-benzoimidazol-1-yl]-phenol;

4-[2-(3,5-dimethyl-isoxazol-4-yl)-benzoimidazol-1-yl)-phenol;

4-[2-(3-bromo-thiophen-2-yl)-benzoimidazol-1-yl]-phenol;

4-(2-isothiazol4-yl-benzoimidazol-1-yl)-phenol;

4-[2-[(4-methyl-isothiazol-5-yl)-benzoimidazol-1-yl]-phenol;

4-[2-(4-methyl-[1,2,3]thiadiazol-5-yl)-benzoimidazol-1-yl]-phenol;

4-[2-(3-chloro-thiophen-2-yl)-benzoimidazol-1-yl]-phenol;

4-[2-(1-ethyl-1H-pyrrol-2-yl)-benzoimidazol-1-yl]-phenol;

4-(2-furan-3-yl-benzoimidazol-1-yl-phenol;

4-[2-(3-methyl-furan-2-yl)-benzoimidazol-1-yl]-phenol;

4-(2-furan-2-yl-benzoimidazol-1-yl)-phenol;

4-[2-(3-ethyl-isoxazol4-yl)-benzoimidazol-1-yl-phenol;

4-[2-(3-cyclopropyl-isoxazol4-yl)-benzoimidazol-1-yl]-phenol;

4-[2-(3-ethyl-5-methyl-isoxazol-4-yl)-benzoimidazol-1-yl]-phenol;

4-[2-(5-methyl-isoxazol-4-yl)-benzoimidazol-1-yl]-phenol;

4-[2-(3-methyl-isoxazol4-yl]-phenol;

4-[2-(2-methyl-thiophen-3-yl)-benzoimidazol-1-yl]-phenol;

4-[2-(2-methyl-furan-3-yl)-benzoimidazol-1-yl]-phenol;

4-[2-(2,5-dimethyl-furan-3-yl)benzoimidazol-1-yl]-phenol;

4-[2-(1-propyl-1H-pyrrol-2-yl)-benzoimidzol-1-yl]-phenol;

4-[2-(1-isopropyl-1H-pyrrol-2-yl)-benzoimidazol-1-yl]-phenol;

3-methyl4-[2-(1-methyl-1H-pyrrol-2-yl)-benzoimidazol-1-yl]-phenol;

4-[2-(3,5-dimethyl-isoxazol-4-yl)-benzoimidazol-1-yl]-3-methyl-phenol;

4-[2-(3-methyl-thiophen-2-yl)-benzoimidazol-1-yl]-phenol;

4-(2-isothiazol-5-yl-benzoimidazol-1-yl)-phenol and N-benzhydryl-4-[1-(4-hydroxy-phenyl)-1H-benzoimidazol-2-yl]-benzamide, and the pharmaceutically accepted salts thereof.

Compounds that are selective or more selective for the ERβ receptor have the advantage that they can be used in treatments specifically designed to target certain tissues containing ERβ receptors. This would avoid unnecessarily agonizing or, antagonizing other receptors in tissue, for example ERα receptors, and would thus avoid potential problems.

The compounds of this invention may be administered to mammals (including humans) orally or parenterally in the conventional form of preparations, such as capsules, microcapsules, tablets, granules, powder, troches, pills, suppositories, injections, suspensions and syrups. Suitable formulations may be prepared by methods commonly employed using conventional organic or inorganic additives, such as excipients, binders, disintegrators, lubricants, flavoring agents, stabilizers, dispersing agents, diluents, preservatives, and a base wax. The amount of the active ingredient in the preparation may be at a level that will exhibit the desired therapeutic effect. The active ingredient may be usually administered once to four times a day with a unit dosage of 0.1 mg to 50 mg in human patients, but the above dosage may be properly varied depending on the age, body weight and medical condition of the patient and the type of administration.

In addition to the compounds as described above, the following compounds were found to have activity as estrogen agonists/antagonists: 4-(5-phenyl-2-trifluoromethyl-3H-imidazol-4-yl)-phenol; 4-[5-[(4-hydroxy-phenyl)2-trifluoromethyl-3-H-imidazol-4-yl]-phenol; 4-[5-[(4-methoxy-phenyl)-2-trifluoromethyl-1H-imidazol-4-yl]-phenol; and 4-(4-phenyl-5-trifluoromethyl-isoxazol-3-yl)-phenol. The 4-(5-phenyl-2-trifluoromethyl-3H-imidazol-4-yl)--phenol, 4-[5[(4-hydroxy-phenyl)2-trifluoromethyl-3-H-imidazol-4-yl]-phenol; 4[5-[(4-methoxy-phenyl)-2-trifluoromethyl-1H-imidazol4-yl]-phenol compounds may be prepared by the methods described in “Preparation and Anti-inflammatory Activity of Some Nonacidic Trisubstituted Imidazoles”, Journal of Medicinal Chemistry, 1974, Vol. 17, No.11. The 4-(4-phenyl-5-trifluoromethyl-isoxazol-3-yl)-phenol compound may be prepared by the methods described in Example 62.

The compounds of the present invention may also be used in combination with other agents to provide sustained therapeutic and prophylactic effects. The compounds of the present invention may be used with other agents including, but not limited to, an anabolic agent; a growth hormone; a growth hormone secretagogue; a prostaglandin agonist/antagonist; a parathyroid hormone; sodium fluoride; or a mixture thereof.

Any prostaglandin agonist/antagonist may be used in combination with the compounds of this invention. The term prostaglandin agonist/antagonist refers to compounds which bind to prostaglandin receptors (e.g., An S. et al., Cloning and Expression of the EP₂ Subtype of Human Receptors for Prostaglandin E₂, Biochemical and Biophysical Research Communicafions, 1993, 197(1):263-270) and mimic the action of prostaglandin in vivo (e.g., stimulate bone formation and increase bone mass). Such actions are readily determined by those skilled in the art of standard assays. Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296. A variety of these compounds are described and referenced below. However, other prostaglandin agonists/antagonists will be known to those skilled in the art. Exemplary prostaglandin agonists/antagonists are disclosed as follows.

Commonly assigned U.S. Pat. No. 3,932,389, the disclosure of which is incorporated herein by reference, discloses 2-descarboxy-2-(tetrazol-5-yl)-11-desoxy-15-substituted-omega-pentanorprostaglandins useful for bone formation activity.

Commonly assigned U.S. Pat. No. 4,018,892, the disclosure of which is incorporated herein by reference, discloses 16-aryl-13,14-dihydro-PGE₂ p-biphenyl esters useful for bone formation activity.

Commonly assigned U.S. Pat. No. 4,219,483, the disclosure of which is incorporated herein by reference, discloses 2,3,6-substituted-4-pyrones useful for bone formation activity.

Commonly assigned U.S. Pat. No. 4,132,847, the disclosure of which is incorporated herein by reference, discloses 2,3,6-substituted-4-pyrones useful for bone formation activity.

U.S. Pat. No. 4,000,309, the disclosure of which is incorporated herein by reference, discloses 16-aryl-13,14-dihydro-PGE₂ p-biphenyl esters useful for bone formation activity.

U.S. Pat. No. 3,982,016, the disclosure of which is incorporated herein by reference, discloses 16-aryl-13,14-dihydro-PGE₂ p-biphenyl esters useful for bone formation activity.

U.S. Pat. No. 4,621,100, the disclosure of which is incorporated herein by reference, discloses substituted cyclopentanes useful for bone formation activity.

U.S. Pat. No. 5,216,183, the disclosure of which is incorporated herein by reference, discloses cyclopentanones useful for bone formation activity.

Sodium fluoride may be used in combination with the compounds of this invention. The term “sodium fluoride” refers to sodium fluoride in all its forms (e.g., slow release sodium fluoride, sustained release sodium fluoride). Sustained release sodium fluoride is disclosed in U.S. Pat. No. 4,904,478, the disclosure of which is incorporated herein by reference. The activity of sodium fluoride is readily determined by those skilled in the art of biological protocols (e.g., see Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-2; Wahner H. W. and Fogelman I., The Evaluation of Osteoporosis: Dual Energy X-Ray Absorptiometry in Clinical Practice., Martin Dunitz Ltd., London 1994, pages 1-296).

Any parathyroid hormone (PTH) may be used in combination with the compounds of this invention. The term parathyroid hormone refers to parathyroid hormone, fragments or metabolites thereof and structural analogs thereof which can stimulate bone formation and increase bone mass. Also included are parathyroid hormone related peptides and active fragments and analogs of parathyroid related peptides (see PCT publication no. WO 94/01460). Such bone anabolic functional activity is readily determined by those skilled in the art of standard assays (e.g., see Eriksen E. F. et al., Bone Histomorphometry, Raven Press, New York, 1994, pages 1-74; Grier S. J. et. al., The Use of Dual-Energy X-Ray Absorptiometry In Animals, Inv. Radiol., 1996, 31(1):50-62; Wahner H. W. and Fogelman I., The Evaluation of Osteoporosis: Dual Energy X-Ray Absorptiometry in Clinical Practice., Martin Dunitz Ltd., London 1994, pages 1-296). A variety of these compounds are described and referenced below. However, other parathyroid hormones will be known to those skilled in the art. Exemplary parathyroid hormones are disclosed in the following references.

“Human Parathyroid Peptide Treatment of Vertebral Osteoporosis”, Osteoporosis Int., 3, (Supp 1):199-203.

“PTH 1-34 Treatment of Osteoporosis with Added Hormone Replacement Therapy: Biochemical, Kinetic and Histological Responses” Osteoporosis Int. 1:162-170.

Any growth hormone or growth hormone secretagogue may be used in combination with the compounds of this invention. The term “growth hormone secretagogue” refers to a compound which stimulates the release of growth hormone or mimics the action of growth hormone (e.g., increases bone formation leading to increased bone mass). Such actions are readily determined by those skilled in the art of standard assays well known to those of skill in the art. A variety of these compounds are disclosed in the following published PCT patent applications: WO 95/14666; WO 95/13069; WO 94/19367; WO 94/13696; and WO 95/34311. However, other growth hormones or growth hormone secretagogues will be known to those skilled in the art.

In particular a preferred growth hormone secretagogue is N-[1(R)-[1,2-Dihydro-1-methanesulfonylspiro[3H-indole-3,4′-piperidin]-1′-yl)carbonyl]-2-(phenylmethyloxy)ethyl]-2-amino-2-methylpropanamide:MK-677.

Other preferred growth hormone secretagogues include

2-amino-N-(2-(3a-(R)-benzyl-2-methyl-3-oxo-2,3,3a ,4,6,7-hexahydro-pyrazolo-[4,3-c]pyridin-5-yl)-1-(R)-benzyloxymethyl-2-oxo-ethyl)-isobutyramide or its L-tartaric acid salt;

2-amino-N-(1-(R)-benzyoxymethyl-2-(3a-(R)-(4-fluoro-benzyl)-2-methyl-3-oxo-2,3,3a,4,6,7-hexahydro-pyrazolo[4,3-c]pyridin 5-yl)-2-oxo-ethyl)isobutyramide;

2-amino-N-(2-(3a-(R)-benzyl-3-oxo-2,3,3a,4,6,7-hexahydro-pyrazolo[4,3-c]pyridin-5-yl)-1-(R)benzyloxymethyl-2-oxo-ethyl)isobutyramide; and

2-amino-N-(1-(2,4-difluoro-benzyloxymethyl)-2-oxo-2-(3-oxo-3a-pyridin-2-ylmethyl-2-(2,2,2-trifluoro-ethyl)-2,3,3a,4,6,7-hexahydro-pyrazolo[4,3-c]pyridin-5-yl)-ethyl)-2-methyl-propionamide.

In a further aspect this invention relates to a kit comprising:

a. an amount of a compound of formula (I) a prodrug thereof or a pharmaceutically acceptable salt of said compound or said prodrug, or a steroisomer or diastereomeric mixture of a compound of formula (I), prodrug or salt and a pharmaceutically acceptable carrier or diluent in a first unit dosage form; p

b. an amount of a second compound an anabolic agent; a growth hormone; a growth hormone secretagogue; a prostaglandin agonist/antagonist, a parathyroid hormone; sodium fluoride; or a mixture thereof; and

c. a container.

Suitable second compounds for use in the kit as defined above are described in the specification above.

It will be recognized that prodrugs and pharmaceutically acceptable salts may be formed from the compounds used as the second compounds in the combinations and kits of the invention. All of such prodrugs and pharmaceutically acceptable salts so formed are within the scope of this invention.

### DETAILED DESCRIPTION OF THE INVENTION

The following reaction schemes illustrate the preparation of the compounds of the present invention. Unless otherwise indicated, the substitutents in the reaction scheme and the discussion that follows are defined as above. The reactants in the following scheme have been renumbered for clarity of discussion.

Before the present compositions and methods are disclosed and described, it is to be understood that this invention is not limited to specific systemic methods or to particular formulations, as such may, of course, vary. It is also to be understood that the terminology used herein is for the purpose of describing particular embodiments only, and is not intended to be limiting.

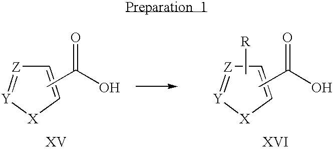

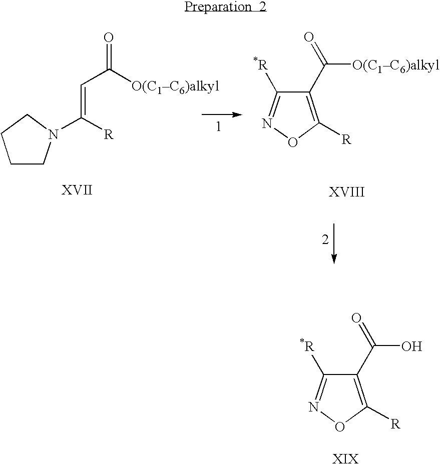

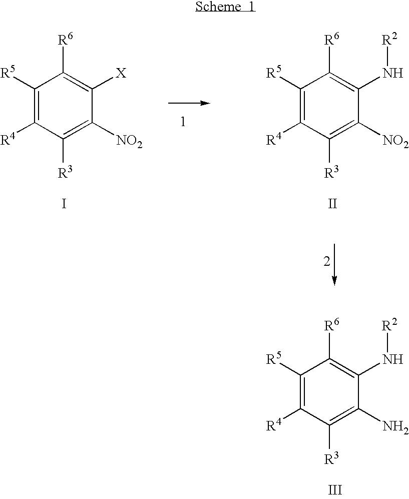

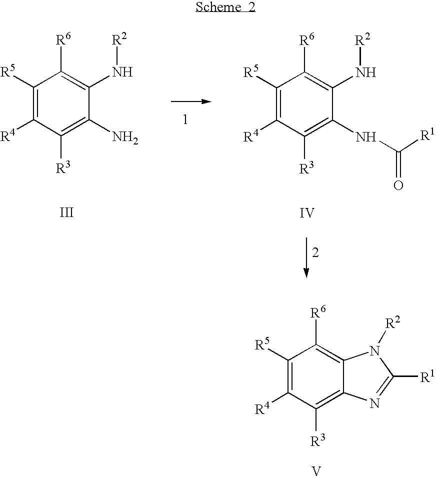

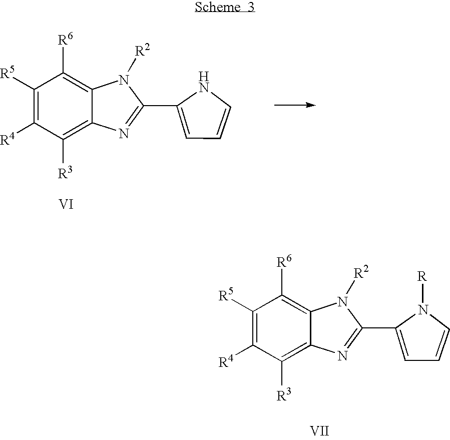

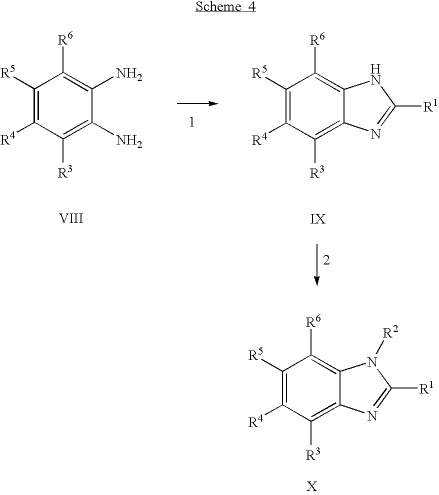

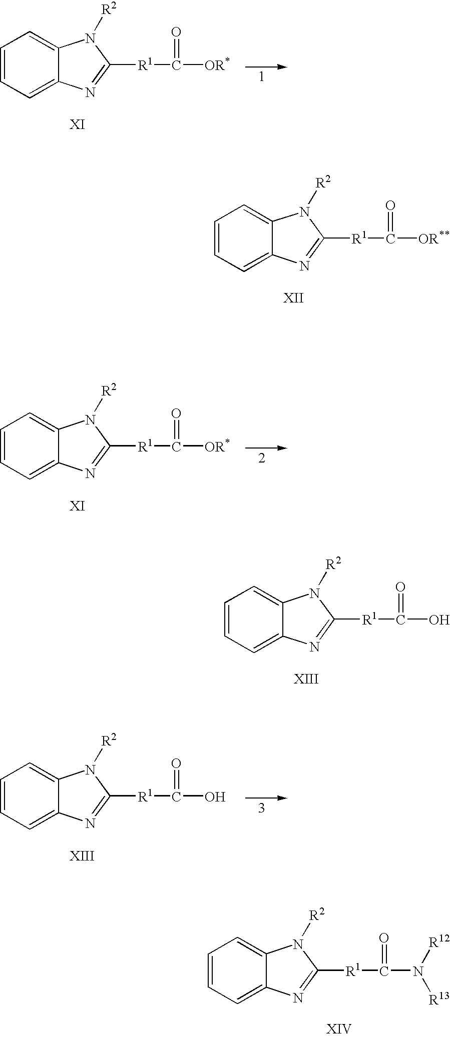

Preparations 1, 2 and 3 describe the preparation of materials that are used to prepare compounds according to the present invention.

As shown in Preparation 1 carboxylic acids which are not commercially available may be synthesized. In the carboxylic acid compound of Formula (XV), the variables X,Y, and Z are each independently nitrogen, oxygen or sulfur. The carboxylic acid compound of Formula (XV) is treated with an excess amount of a strong base, such as lithium diisopropylamide (LDA) or butyl lithium, in an inert solvent, such as tetrahydrofuran (THF), dimethyl ether (DME), dioxane, or a mixture thereof at a temperature of from about −78° C. to about 100° C., preferably about room temperature, for a period between about 1 hour to about 24 hours, preferably about 12 hours and is then alkylated with an alkyl halide, at a temperature of from about −78° C. to about 100° C., preferably about room temperature, for a time period of between about 1 hour to about 24 hours, preferably about 12 hours, to give carboxylic acid of Formula (XVI).

As shown in preparation 2, the 3,5 disubstituted carboxy isoxazoles may be prepared by treatment of a vinylogous carbamate with a nitrile oxide compound such as acetonitrile oxide, propionitrile or cyclopropane carbonitrile to give esters (XVIII). Nitrile oxides may be synthesized by methods known to those of skill in the art and as described in Journal of the American Chemical Society 1960, 82, 533942; and Journal of Medicinal Chemistry, 1976, 19, 562-565, which are incorporated by reference in their entirety. The ester may be converted to the corresponding carboxylic acid compound of Formula (XIX) by treatment with lithium hydroxide (LiOH), sodium hydroxide (NaOH) or potassium hydroxide (KOH) in a solvent such as methanol, water, ethanol, or a THF/water mixture, at a temperature of from about 0° C. to about 100° C., preferably at about room temperature.

As shown in preparation 3, reaction 1, the nitrobenzene compound of Formula (I) wherein X is a halogen (including chlorine, fluorine, or bromine, preferably fluorine), is reacted with an amine compound having the Formula NH₂R² to produce a nitroaniline compound of Formula (II). For example if R² is a phenyl substituent, the amine could be chosen to be an aniline compound. The reaction is conducted in the presence of a base, such as potassium carbonate, potassium tert-butoxide, powdered sodium hydroxide, or powdered potassium hydroxide to give the corresponding nitroaniline compound of Formula (II). The reaction may be conducted at a temperature between about room temperature to about 200° C., preferably at about 160° C., for a time period from between about 2 to 24 hours, preferably about 12 hours. The reaction may be conducted neat or in a solvent. Suitable solvents include dimethyl sulphoxide (DMSO), dimethylformamide (DMF) or a mixture thereof.

As shown in reaction 2 of preparation 3, the nitroaniline compound of Formula (II) is reduced to the amine functionality upon treatment with hydrogen gas in the presence of a metal such as palladium, platinum or nickel to give aniline compounds of Formula (Ill). Either pure metal or metal on carbon, such as palladium on carbon, nickel on carbon, or platinum on carbon may be used. The reaction is conducted at a temperature between about 0° C. to about 100° C., preferably room temperature, for a time period between about 1 to about 24 hours, preferably about 12 hours.

As shown in reaction scheme 1, the aniline compound of Formula (III), prepared according to the method of Preparation 3, is coupled with carboxylic acid compounds having an appropriate R¹ substituent in the presence of an appropriate coupling agent, such as 1-propanephosphonic acid cyclic anhydride (PPM), 1-(3-dimethylaminopropyl)-3-ethylcarbodiimide hydrochloride (EDC), or a mixture thereof, and a catalytic amount of an additive such as 1-hydroxybenzotriazole (HOBt) or 4-dimethylaminopyridine (DMAP) at a temperature between about 0° C. to about 60° C., preferably about room temperature, for a time period between about 1 to about 36 hours, preferably about 12 hours. Carboxylic acids may be obtained commercially or may be prepared analogously according to the methods described in Preparation 1. Alternatively, the aniline compounds of Formula (III) may be treated with an acid chloride compound or an acid anhydride compound of the corresponding carboxylic compounds having the appropriate R¹ substituent, the reaction with the acid chloride or acid anhydride being conducted in the presence of a tertiary amine base such as triethylamine or 4-dimethylaminopyridine (DMAP) to give amide compounds of Formula (IV). Suitable acid chloride and acid anhydrides are commercially available or can be prepared from corresponding carboxylic acids by procedures analogous to those described in reference to Preparation 1. Any unreacted aniline compounds of Formula (III) from the aforementioned reactions may be optionally removed by treatment with a scavenger reagent, such as polymer supported isocyanate.

The amide compound of Formula (IV) is cyclodehydrated upon treatment with an acid, such as acetic acid or hydrochloric acid, at temperature from about room temperature to about 100° C., preferably at about 75° C., to give the benzimidazole compounds (V).

In the reaction schemes above, if R¹ and/or R² have a hydroxyl substituent, or if R³, R⁴, R⁵ or R⁶ are hydroxyl, it is preferable to protect the hydroxyl substituents through the use of protecting groups for all hydroxyls. Protection may be effected by treatment of the compound containing the hydroxyl substituent with a strong base such as sodium hydride (NaH), sodium hexamethyldisilazide (NaHMDS) or potassium hexamethyldisilazide (KHMDS) and reaction with an electrophile such as an alkyl halide, such as methyl iodide or benzyl bromide. The reaction may take place in an inert solvent, such as diethyl ether, dimethylformamide (DMF), tetrahydrofuran (THF), toluene or a mixture thereof, at a temperature of from about 0° C. to about 100° C., preferably at room temperature. Removal of the benzyl protecting groups may be effected by treatment with hydrogen, in the presence of a metal catalyst, such as platinum, nickel or palladium, preferably palladium, in an inert solvent, such as tetrahydrofuran (THF), ethanol (EtOH) or methanol (MeOH), preferably EtOH at a temperature of room temperature to 100° C., preferably at room temperature. Methyl ether protecting groups can be removed by treatment with boron tribromide in an inert solvent such as methylene chloride or 1,2 dichloroethane, preferably methylene chloride at a temperature of between about −78° C. to reflux, preferably at about 0° C. Preferred protecting groups are methyl and benzyl ethers. Tetrahydropyranyl (THP) protecting groups may also be used. The THP protecting group may be introduced using dihydropyran with a suitable acid catalyst, such as sulphuric acid, para-toluene sulfonic acid (TsOH) or pyridinium para-toluene sulphonate (PPTS), in an inert solvent, such as methylene chloride, THF or 1,2 dichlorethane, preferably methylene chloride. The reaction can be run at a temperature of from about 0 to about 85° C., preferably at room temperature. The THP group may be removed by treatment with and acid such as acetic acid, trifluoroacetic acid (TFA), hydrochloric acid (HCl), para-toluene sulfonic acid (TsOH), PPTS or magnesium bromide (MgBr₂) in the presence of a protic solvent such as trifluouroacetic acid, water, methanol or ethanol. Triethylsilane may optionally be added to the reaction. Greene, T. W.; Wuts, P. G. M. Protective Groups in Organic Synthesis, 2^{nd} edition, John Wiley and Sons, Inc. New York, 1991, incorporated herein by reference in its entirety, provides a general description of protecting groups and their uses.

As shown in reaction scheme 2, the pyrrolyl compound of Formula (VII) is prepared from the pyrrole compound of Formula (VI) by treatment with a strong base, such as potassium tert-butoxide or potassium hexamethyldisilazide (KHDMS), in the presence of a suitable crown ether, such as 18-crown-6 for potassium bases, 15-crown-5 for sodium bases, and 12-crown4 for lithium bases, at a temperature of from about −78° C. to room temperature, preferably about 0° C. for a time period of from about 30 minutes to about 24 hours, preferably about 1 hour. This is followed by treatment with an alkyl halide having the desired alkyl substituent, such as methyl iodide in an inert solvent such as THF, DMF, dioxane, dimethoxy ethane (DME) or a mixture thereof, at a temperature of from about −78° C. to about 100° C., preferably at about room temperature, for a time period from about 1 hour to about 72 hours, preferably about 24 hours. Alternatively, pyrrolyl compounds may be analogously prepared by the methods described in schemes 1 and 2, above.

As shown in reaction scheme 3, reaction 1, the aniline compound of Formula (VIII) may be treated with an acid chloride or acid anhydride in the presence of a tertiary amine base such as triethylamine or dimethylamino pyridine (DMAP) with subsequent cyclodehydration to give a benzimidazole (IX). The reaction is conducted in an inert solvent such as methylene chloride, THF, DMF or a mixture thereof, preferably methylene chloride. Alternatively, the aniline compound may be treated with a carboxylic acid and an appropriate coupling agent, as described in reference to reaction scheme 1 with subsequent cyclodehydration. Carboxylic acids which are not available commercially may be prepared according to preparation 1. Suitable acid chloride and acid anhydrides are commercially available or can be prepared from corresponding carboxylic acids by procedures analogous to those described in reference to Preparation 1. The reaction is conducted at a temperature of from about 0° C. to about 100° C. at a time of from about 1 to about 48 hours, preferably about 12 hours. As shown in reaction scheme 3, reaction 2, the benzimidazole compound may be arylated by treatment with an aromatic or heteroaromatic halide in the presence of a suitable metal catalyst such as tris(dibenzylideneacetone)dipalladium(0) with the appropriate additives, such as 1,10 phenanthroline, copper(l)trifluoromethane sulfonate benzene, cesium carbonate, or a mixture thereof to produce a compound of Formula (X). By “arylated” it is meant that the R² substituent in the compound of Formula (X) is an aryl or heteroaryl groups, such as phenyl or thienyl. The reaction may be conducted in a suitable solvent, such as xylene, DMF, or a mixture thereof, at a temperature of from about 0 to about 165° C., preferably about 135° C. for a time period of between about 1 to about 72 hours, preferably about 48 hours.

Alternatively, the benzimidazole compound of Formula (IX) may be arylated by treatment with a boronic acid in the presence of a suitable catalyst, such as copper(II) acetate to give an arylated compound of Formula (X). The reaction may be conducted in the presence of a base, such as pyridine, Et₃N or 1,4 diazobicylo[2.2.2]octane, in an inert solvent, such as toluene, methylene chloride or a mixture thereof, at a temperature of from about 0° C. to about 100° C., preferably about room temperature. The reaction may be conducted for a time period of from about 1 hour to about 72 hours, preferably 36 hours.

As shown in reaction scheme 4, the benzimidazole ester of Formula (XII) may be prepared from a benzimidazole alkyl ester compound of Formula (XI) by transesterification and -subsequent deprotection. In one embodiment, methyl esters are the benzimidazole alkyl ester compounds of Formula (XI). Transesterification may be accomplished by treatment with boron tribromide (BBr₃) in a solvent, such as methylene chloride, chloroform, 1,2 dichloroethane or a mixture thereof, followed by the addition of the alcohol having the desired alkyl substituent, for the transesterification.

Alternatively, the benzimidazole alkyl ester compound of Formula (XI) may be hydrolyzed to a carboxylic acid compound of Formula (XIII) and coupled with the appropriate alcohol under conditions as described in reference to the coupling reaction as described in reference to scheme 1, reaction 1, except using the appropriate alcohol in place of the aniline.

The benzimidazole amide compound of Formula (XIV) may be prepared from the carboxylic acid compound of Formula (XIII) by coupling with an appropriate amine compound under conditions as described in reference to the coupling reaction as described in reference to reaction scheme 1.

The subject invention also includes isotopically-labelled compounds, which are identical to those recited in Formulas (I) but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H,¹³C, ¹⁴C, ¹⁵N, ¹⁸O, ¹⁷O, ³¹P, ³²P, ³⁵S, ¹⁸F, and ³⁶Cl, respectively. Compounds of the present invention (including the prodrugs thereof and the pharmaceutically acceptable salts of the compounds and the prodrugs) which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be preferred in some circumstances. Isotopically labelled compounds of Formula (I) of this invention and prodrugs thereof can generally be prepared by carrying out the procedures disclosed in the Schemes and/or in the Examples below, by substituting a readily available isotopically labelled reagent for a non-isotopically labelled reagent.

As mentioned, compounds of the present invention may act as antagonists or agonists. The antagonist/agonist activity of the compounds may be determined by any method known in the art. For example, estrogenic activity in human breast cancer MCF7 cells and primary rat granulosa cells may be assessed by transient transfection of an estrogen responsive ERE3-TK-lux luciferase reporter vector essentially as has been described previously in other cell backgrounds, as in Petersen D N, Tkalcevic G T, Koza-Taylor P H, Turi T G & Brown T A (1998) Identification of estrogen receptor β2, a functional variant of estrogen receptor expressed in normal rat tissue. Endocrinology 139: 1082-1092, incorporated herein by reference in its entirety. The MCF7 cell activity was considered to be mediated through ERα and the granulosa activity was considered to be mediated through ERβ.MCF7 cells may be obtained from ATCC (Manassas, Va.) and transfected with Lipofectamine Plus (Gibco/BRL, Rockville, Md.) as described by the manufacturers. Luciferase may be measured 24 hours after compound addition. Primary rat granulosa cells may be isolated and transfected with ERE3-TK-lux as described in O'Brien M L, Park K, In Y, & Park-Sarge O -K (1999) Characterization of estrogen receptor-β (ERβ) messenger ribonucleic acid and protein expression in rat granulosa cells. Endocrinology 140: 4530-4541, incorporated herein by reference in its entirety.

The invention has been described in detail with particular reference to specific embodiments thereof, but it will be understood that various modifications can be effected within the scope of the invention.

Other features and advantages will be apparent from this description and claims that describe the invention.

### EXAMPLES

The following examples are set forth to provide those of ordinary skill in the art with a complete description of how the compositions of matter and methods claimed herein are made and evaluated, and are not intended to limit the scope of what the inventors regard as their invention. The activity of these compounds as receptor. antagonists for ERα and ERβ may be demonstrated by the assay for receptor binding activity.

Assay for Estrogen Receptor Binding Activity

cDNA cloning of human ERα and ERβ: The coding region of human ERα was cloned by reverse transcriptase polymerase chain reaction (RT-PCR) from human breast cancer cell mRNA using EXPAND High Fidelity PCR System according to manufacturer's instructions (Boehringer-Mannheim,. Indianapolis, Ind.). The coding region of human ERβ was cloned by RT-PCR from human testes and pituitary mRNA using EXPAND High Fidelity PCR System according to manufacturer's instructions (Boehringer-Mannheim, Indianapolis, Ind.). PCR products were cloned into pCR2.1 TA Cloning Kit (Invitrogen, Carlsbad, Calif.) and sequenced. Each receptor coding region was subcloned into the mammalian expression vector pcDNA3 ((Invitrogen, Carlsbad, Calif.).

Mammalian cell expression. Receptor proteins were overexpressed in 293T cells. These cells, derived from HEK293 cells (ATCC, Manassas, Va.), have been engineered to stably express large T antigen and can therefore replicate plasmids containing a SV40 origin of replication to high copy numbers. 293T cells were transfected with either hERα-pcDNA3 or hERP-pcDNA3 using lipofectamine as described by the manufacturer (Gibco/BRL, Bethesda, Md.). Cells were harvested in phosphate buffered saline (PBS) with 0.5 mM EDTA at 48 h post-transfection. Cell pellets were washed once with PBS/EDTA. Whole cell lysates were prepared by homogenization in TEG buffer (50 mM Tris pH 7.4, 1.5 mM EDTA, 50 mM NaCl, 10% glycerol, 5 mM DTT, 5 μg/ml aprotinin, 10 μg/ml leupeptin, 0.1 mg/ml Pefabloc) using a dounce homogenizor. Extracts were centrifuged at 100,000×g for 2 h at 4C. and supernatants were collected. Total protein concentrations were determined using BioRad reagent (BioRad, Hercules, Calif.).

Competition binding assay. The ability of various compounds to inhibit [³H]-estradiol binding was measured by a competition binding assay using dextran-coated charcoal as has been described (Leake R E, Habib F 1987 Steroid hormone receptors: assay and characterization. In: B. Green and R. E. Leake (eds)., Steroid Hormones a Practical Approach. IRL Press Ltd, Oxford. 67-92.) 293T cell extracts expressing either hERα or hERβ were incubated in the presence of increasing concentrations of competitor and a fixed concentration of [³H]-estradiol (141 Ci/mmol, New England Nuclear, Boston, Mass.) in 50 mM TrisHCl pH 7.4, 1.5 mM EDTA, 50 mM NaCl, 10% glycerol, 5 mM DTT, 0.5 mg/mL β-lactoglobulin in a final volume of 0.2 mL. All competitors were dissolved in dimethylsulfoxide. The final concentration of receptor was 50 pM with 0.5 nM [³H]-estradiol. After 16 h at 4C., dextran-coated charcoal (20 μL) was added. After 15 min at room temperature the charcoal was removed by centrifugation and the radioactive ligand present in the supernatant was measured by scintillation counting. All reagents were obtained from Sigma (St. Louis, Mo.) unless otherwise indicated. Binding assay results are IC₅₀ values and are reported in nanomoles (nmol) below each compound in the examples that follow.

General Experimental Procedures

NMR spectra were recorded on a Varian Unity 400 spectrometer (Varian Co., Palo Alto, Calif.) at about 23° C. at 400 MHz for proton nuclei. Chemical shifts are expressed in parts per million. The peak shapes are denoted as follows: s, singlet; d, doublet; t, triplet; q, quartet; m, multiplet; bs, broad singlet. Atmospheric pressure chemical ionization (APCI) mass spectra were obtained on a Fisons Platform II Spectrometer (Micromass Inc., Beverly, Mass.). Where the intensity of chlorine or bromine-containing ions are described the expected intensity ratio was observed (approximately 3.1 for ³⁵Cl/³⁷C₁-containing ions) and 1:1 for ⁷⁹Br/⁸¹Br-containing ions) and the intensity of only the lower mass ion is given (except where stated).

Medium pressure chromatography was performed using a Biotage purification system (Biotage, Dyax Corporation, Charlottesville, Va.) under nitrogen pressure. Flash chromatography was performed with either Baker Silica Gel (40 μm) (J. T. Baker, Phillipsburg, N.J.) or Silica Gel 60 (EM Sciences, Gibbstown, N.J.) in glass columns under low nitrogen pressure. Radial Chromatography was performed using a Chromatotron (model 7924T, Harrison Research, Palo Alto, Calif.). Preparative Chromatography was performed using Analtech Uniplates Silica Gel GF (20×20 cm) (Analtech, Inc. Newark, Del.). Dimethylformamide (DMF), tetrahydrofuran (THF), and dichloromethane (CH₂Cl₂) used as reaction solvents were the anhydrous grade supplied by Aldrich Chemical Company (Milwaukee, Wis.). The term “concentrated” refers to removal of solvent at water aspirator pressure on a rotary evaporator. The term “EtOAc” means ethyl acetate. The abbreviation ‘h’ stands for hours. The term “TBAF” refers to tetrabutylammonium fluoride. The term “DMAP” refers to dimethylaminopyridine. The terms “dichloromethane” and “methylene chloride” are synonymous and are used interchangeably throughout this description and in the Examples and Preparations.

Abbreviations

Abbreviations used in the following examples and preparations include:

1,2 DCE 1,2-Dichloroethane

d Doublet

dd Double Doublet

cat. catalytic

DMAP 4-Dimethylamino Pyridine

DMSO dimethyl sulphoxide

EDC 1-(3-Dimethylaminopropyl)-3-ethylcarbodiimide Hydrochloride

EtOAc Ethyl Acetate

EtOH Ethyl Alcohol or Ethanol

Et₂O Ethyl Ether

Et₃N Triethylamine

HOBt 1-Hydroxybenzotriazole

HOLC High Pressure Liquid Chromatography

h or hr Hour(s)

m Multiplet

KHMDS Potassium hexamethylsilazide

LDA Lithium Di-isopropylamide

MeOH Methyl Alcohol or Methanol

min Minute(s)

MS Mass Spectrometry

n-BuLi n-Butyl Lithium

NCS N-Chlorosuccinimde

NMR Nuclear Magnetic Resonance

PLC Preparative thin layer chromatography

PPAA 1-Propanephosphonic Acid Cyclic Anhydride

p.s.i. pounds per square inch

q Quartet

RT (or rt) room temperature (about 20-25° C.)

s Singlet

sat. Saturated

t Triplet

TBAF Tetrabutyl Ammonium Fluoride

TLC Thin Layer Chromatography

TFA Trifluoroacetic Acid

THF Tetrahydrofuran

All receptor binding data is in nM except where stated.

### Example 1

4-(2-Thiophen-3-yl-benzoimidazol-1-yl)-phenol

Step A

(4-Methoxy-phenyl)-(2-nitro-phenyl)-amine

1-Fluoro-2-nitrobenzene (37.6 g, 28.0 ml, 0.266 mmol), p-anisidine (32.8 g, 0.266) and K₂CO₃ (55.0 g, 0.399 mol) were combined in flask and heated at 160° C. overnight under an atmosphere of N₂. The mixture was cooled to ca. 90° C. and water (200 ml) was added slowly to the reaction. The mixture was partitioned with EtOAc (1L). The remaining solid was stirred for 45 minutes in EtOAc (200 ml), MeOH (200 ml) and water (200 ml) until complete dissolution occurred. The mixture was extracted with EtOAc (3×400 ml), the aqueous washings were combined and further extracted with EtOAc (2×200 ml). All of the organic extracts were combined and washed with brine (600 ml), dried (MgSO₄), filtered and concentrated by vacuum. The solid residue was recrystallised from hot hexanes (1L) to give (4-methoxy-phenyl)-(2-nitro-phenyl)-amine (46.07 g, 0.253 mol, 95%). MS (M+1) 245. ¹H NMR (CDCl₃) δ_{H} 9.40 (1H, br. s), 8.18 (1H, m), 7.23 (3H, overlapping m), 6.97 (3H, overlapping m), 6.70 (1H, m) and 3.84 (3H, s).

Step B

N-(4-Methoxy-phenyl)-benzene-1,2-diamine di-hydrochloride salt

A mixture of (4-methoxy-phenyl)-(2-nitro-phenyl)-amine (55.79 g, 0.23 mol), 4M HCl in dioxane (250 ml, 1 mol), and 10% Pd/C (6 g) in MeOH (800 ml) was hydrogenated on a Parr shaker at 50 p.s.i. for 3 hours. The mixture was filtered through diatomaceous earth and the pad was washed with hot MeOH (2×200 ml). The filtrate was concentrated to ca. 400 ml in volume and the resulting solid was filtered. A second crop of solid was obtained from the filtrate, which was then washed with Et₂O. The filtrate was neutralized with sat. NaHCO₃ and extracted with CH₂Cl₂ (3×200 ml), the organic extracts were combined, dried (MgSO₄) and concentrated by vacuum. The resultant oil was dissolved in Et₂O and 1M HCl in Et₂O (100 ml) was added and the solid was filtered off. The solids were combined to give N-(4-methoxy-phenyl)-benzene-1,2-diamine di-hydrochloride salt (55.1 g, 0.192 mol, 83%). MS (M+1) 215. ¹H NMR (CD₃OD) δ_{H} 7.33 (2H, m), 7.17 (1H, dd), 7.02 (1H, dt), 6.95 (2H, m), 6.88 (2H, m) and 3.76 (3H, s).

Step C

Thiophene-3-carboxylic acid [2-(4-methoxy-phenylamino)-phenyl]-amide

To a solution of N-(4-,methoxy-phenyl)-benzene-1,2-diamine di-hydrochloride (0.15 g, 0.52 mmol), 3-thiophenecarboxylic acid (0.10 g, 0.78 mmol), Et₃N (0.264 g, 0.36 ml, 2.615 mmol) and DMAP (cat.) was added PPAA as a 50% solution in EtOAc (0.332 g, 0.314 ml, 1.046 mmol). The reaction was stirred at room temperature overnight, then sat NaHCO₃ was added and the mixture was extracted with CH₂Cl₂ (3×1 ml). The organics were combined, dried (Na₂SO₄), filtered and concentrated under a stream of nitrogen to give thiophene-3-carboxylic acid [2-(4-methoxy-phenylamino)-phenyl]. MS (M+1) 325.

Step D

1-(4-Methoxy-phenyl)-2-thiophen-3-yl-1H-benzoimidazole

A solution of thiophene-3-carboxylic acid [2-(4-methoxy-phenylamino)-phenyl]-amide in AcOH (3 ml) was heated at 80° C. overnight. The AcOH was removed by vacuum and the residue was taken up in CH₂Cl₂ (5 ml). Sat. NaHCO₃ was added so that the pH of the aqueous solution >7. The layers were separated and the mixture was further extracted with CH₂Cl₂ (2×5 ml). The organics were combined, filtered thorough a 20 μm filter (Alltech) and concentrated under a stream of N₂ to give 1-(4-methoxy-phenyl)-2-thiophen-3-yl-1H-benzoimidazole which was used without purification. MS (M+1) 307.

Step E 4-(2-Thiophen-3-yl-benzoimidazol-1-yl)-phenol

To a-solution of 1-(4-Methoxy-phenyl)-2-thiophen-3-yl-1H-benzoimidazole in CH₂Cl₂ (2 ml) cooled to −78° C. was added BBr₃ as a 1.OM solution in CH₂Cl₂ (2.5 ml, 2.5 mmol). The reaction stirred overnight, slowly warming to room temperature. The solution was re-cooled to −78° C. and MeOH (2 ml) was added. The solution was allowed to warm to room temperature and diluted with CH₂Cl₂ (10 ml). Saturated NaHCO₃ (20 ml) was added (ensuring the pH was between 7 and 9). The mixture was filtered through a 20 μm filter (Alltech) and the resultant solution was dried (MgSO₄), re-filtered and concentrated under a stream of N₂. The mixture was recrystallised from hot MeOH to give 4-(2-Thiophen-3-yl-benzoimidazol-1-yl)-phenol MS (M+1)⁺ 293; ¹H NMR (DMSO-d₆) δ_{H} 7.67 (d, 1H), 7.53-7.55 (m, 1H), 7.32-7.33 (m, 1H), 7.15-7.25 (m, 5H), 7.00 (d, 1H), 6.91-6.95 (m, 2H).

### Example 2

4-(2-Thiophen-2-yl-benzoimidazol-1-yl)-phenol

Step A

Thiophene-2-carboxylic acid [2-(4-methoxy-phenylamino)-phenyl]-amide

Thiophene-2-carboxylic acid [2-(4-methoxy-phenylamino)-phenyl]-amide was prepared in an analogous procedure to that as described in example 1 step C except that 2-thiophenecarboxylic acid was used instead of 3-thiophenecarboxylic acid (0.10 g, 0.78 mmol). MS (M+1) 325.

Step B

1-(4-Methoxy-phenyl)-2-thiophen-2-yl-1H-benzoimidazole

1-(4-methoxy-phenyl)-2-thiophen-2-yl-1H-benzoimidazole was prepared in an analogous procedure to that as described in example 1 step D except that thiophene-2-carboxylic acid [2-(4-methoxy-phenylamino)-phenyl]-amide was used instead of thiophene-3-carboxylic acid [2-(4-methoxy-phenylamino)-phenyl]-amide. MS (M+1) 307.

4-(2-Thiophen-2-yl-benzoimidazol-1-yl)-phenol

4-(2-Thiophen-2-yl-benzoimidazol-1-yl)-phenol was prepared in an analogous procedure to that as described in example 1 step E except that 1-(4-methoxy-phenyl)-2-thiophen-2-yl-1H-benzoimidazole was used instead of 1-(4-Methoxy-phenyl)-2-thiophen-3-yl-1H-benzoimidazole. MS (M+1)⁺ 293; ¹H NMR (CD₃OD) δ_{H} 7.68 (d, 1H), 7.53 (d, 1H), 7.22-7.31 (m, 4H), 7.06-7.10 (m, 2H), 6.99-7.02 (m, 3H).

### Example 3

4-[2-(1-Methyl-1H-pyrrol-2-yl)-benzoimidazol-1-yl]-phenol

1-Methyl-1H-pyrrole-2-carboxylic acid [2-(4-methoxy-phenylamino)-phenyl]-amide

1-Methyl-1H-pyrrole 2-carboxylic acid [2-(4-methoxy-phenylamino)-phenyl]-amide was prepared in an analogous procedure to that as described in example 1 step C except that N-methylpyrrole-2-carboxylic acid (0.098 g, 0.78 mmol) was used instead of thiophene-2-carboxylic acid. MS. (M+1) 322

1-(4-Methoxy-phenyl)-2-(1-methyl-1H-pyrrol-2-yl)-1H-benzoimidazole

1-(4-Methoxy-phenyl)-2-(1-methyl-1H-pyrrol-2-yl)-1H-benzoimidazole was prepared in an analogous procedure to that as described in example 1 step D except that 1-methyl-1H-pyrrole-2-carboxylic acid [2-(4-methoxy-phenylamino)-phenyl]-amide was used instead of thiophene-3-carboxylic acid [2-(4-methoxy-phenylamino)-phenyl]-amide. MS (M+1) 304.

4-[2-(1-Methyl-1H-pyrrol-2-yl)-benzoimidazol-1-yl]-phenol

4-[2-(1-Methyl-1H-pyrrol-2-yl)-benzoimidazol-1-yl]-phenol was prepared in an analogous procedure to that as described in example 1 step E except that 1-(4-methoxy-phenyl)-2-(1-methyl-1H-pyrrol-2-yl)-1H-benzoimidazole was used instead of 1-(4-Methoxy-phenyl)-2-thiophen-3-yl-1H-benzoimidazole. MS (M+1)+290; ¹H NMR (CD₃OD) δ_{H} 7.80 (d, 1H), 7.51-7.57 (t, 1H), 7.47-7.51 (t, 1H), 7.37-741 (m, 1H), 7.28 (m, 2H), 7.02-7.06 (m, 1H), 6.95 (m, 2H), 6.31-6.33 (m, 1H), 6.15 (m, 1H), 3.72 (s, 3H).

### Example 4

4-r2-(3,5-Dimethyl-isoxazol-4-yl)-benzoimidazol-1-yl]-phenol

Step A

3,5-Dimethyl-isoxazole-4-carboxylic acid [2-(4-methoxy-phenylamino)-phenyl]-amide

3,5-Dimethyl-isoxazole-4-carboxylic acid [2-(4-methoxy-phenylamino)-phenyl]-amide was prepared in a procedure analogous to that described in example 1 step C except that 3,5-dimethylisoxazol-4-carboxylic acid (0.083 g, 0.59 mmol) was used instead of 3-thiophenecarboxylic acid MS (M+1) 338.

2-(3,5-Dimethyl-isoxazol-4-yl)-1-(4-methoxy-phenyl)-1H-benzoimidazole

2-(3,5-Dimethyl-isoxazol-4-yl)-1-(4-methoxy-phenyl)-1H-benzoimidazole was prepared in a procedure analogous to that as described in example 1 step D except that 3,5-dimethyl-isoxazole4-carboxylic acid [2-(4-methoxy-phenylamino)-phenyl]-amide was used instead of thiophene-3-carboxylic acid [2-(4-methoxy-phenylamino)-phenyl]-amide. MS (M+1) 320.

4-[2-(3,5-Dimethyl-isoxazol-4-yl)-benzoimidazol-1-yl]-phenol

To a solution of 2-(3,5-dimethyl-isoxazol-4-yl)-1-(4-methoxy-phenyl)-1H-benzoimidazole in CH₂Cl₂ (2 ml) cooled to −78° C. was added BBr₃ as a 1.0M solution in CH₂Cl₂ (1.04 ml, 1.04 mmol). The reaction was stirred overnight slowly warming to room temperature. The solution was re-cooled to −78° C. and MeOH (3 ml) was added. The solution was allowed to warm to room temperature and was diluted was EtOAc. The organic solution was washed with sat. NaHCO₃ (2×20 ml), dried (MgSO₄), filtered and concentrated by vacuum. The residue recrystallised from hot EtOAc/hexanes to give 4-[2-(3,5-dimethyl-isoxazol-4-yl)-benzoimidazol-1-yl]-phenol (0.025 g, 0.0812 mmol). MS (M+1)306; ¹H NMR (CD₃OD) δ_{H} 7.72-7.74 (m, 1H), 7.31-7.36 (m, 3H), 7.14-7.17 (m, 2H), 6.89-6.91 (m, 2H), 2.20 (s, 3H), 2.01 (s, 3H).

### Example 5

4-[2-(3-Bromo-thiophen-2-yl)-benzoimidazol-1-yl]-phenol

Step A

3-Bromo-thiophene-2-carboxylic acid [2-(4-methoxy-phenylamino)-phenyl]-amide

To a solution of N-(4-methoxy-phenyl)-benzene-1,2-diamine di-hydrochloride (0.15 g, 0.52 mmol), (0.16, 0.78 mmol) of 3-bromothiophene-2-carboxylic acid, Et₃N (0.264 g, 0.36 ml, 2.615 mmol) and DMAP (cat) was added PPAA as a 50% solution in EtOAc (0.332 g, 0.314 ml, 1.046 mmol). The reaction was stirred at room temperature overnight. Polymer supported isocyanate (Argonaut technologies, 0.150 g, loading 1.70 mmol/g) were added and the mixture was stirred at room temperature for 6 hours. The solids were removed by filtration and the reaction was concentrated under a stream of nitrogen to give 3-bromo-thiophene-2-carboxylic acid [2-(4-methoxy-phenylamino)-phenyl]-amide, which was used without purification. MS (M+1) 402 and 404(bromine isotope pattern).

2-(3-Bromo-thiophen-2-yl)-1-(4-methoxy-phenyl)-1H-benzoimidazole

A solution of 3-bromo-thiophene-2-carboxylic acid [2-(4-methoxy-phenylamino)-phenyl]-amide in AcOH (3 ml) was heated at 80° C. overnight. The solution was cooled to room temperature and neutralized with sat. NaHCO₃. The mixture was partitioned with CH₂Cl₂, filtered through a 20 μm filter (Alltech) and concentrated under a stream of N₂ to give 2-(3-bromo-thiophen-2-yl)-1-(4-methoxy-phenyl)-1H-benzoimidazole. MS (M+1) 386 and 384 (bromine isotope pattern).

4-[2-(3-Bromo-thiophen-2-yl)-benzoimidazol-1-yl]-phenol

4-[2-(3-Bromo-thiophen-2-yl)-benzoimidazol-1-yl]-phenol was prepared in a procedure analogous to that as described in example 1 step E except that 2-(3-bromo-thiophen-2-yl)-1-(4-methoxy-phenyl)-1H-benzoimidazole was used instead of 1-(4-methoxy-phenyl)-2-thiophen-3-yl-1H-benzoimidazole. MS (M+1) 372,371 (bromine isotope pattern); ¹H NMR (CD₃OD) δ_{H} 7.75 (m, 1H), 7.62-7.63 (m, 1H), 7.29-7.34 (m, 3H), 7.13-7.15 (m, 2H), 7.03-7.04 (m, 1H), 6.82-6.84 (m, 1H).

### Example 6

4-(2-Isothiazol-4-yl-benzoimidazol-1-yl)-phenol

Isothiazole-4-carboxylic acid can be prepared according to the procedure of H. P. Benschop, A. M. Oosten, D. H. J. M. Platenburg and C. Van Hooidonk J. Med. Chem. 1970,13(6), 1208.

Isothiazole-4-carboxylic acid [2-(4-methoxy-phenylamino)-phenyl]-amide

Isothiazole-4-carboxylic acid [2-(4-methoxy-phenylamino)-phenyl]-amide was prepared in a procedure analogous to that described in example 5 step A. except that isothiazole-4-carboxylic acid (0.068 g, 0.523 mmol) was used instead of 3-bromothiophene-2-carboxylic acid.

2-Isothiazol-4-yl-1-(4-methoxy-phenyl)-1H-benzoimidazole

2-Isothiazol-4-yl-1-(4-methoxy-phenyl)-1H-benzoimidazole was prepared in a procedure analogous to that described in example 1 step D except isothiazole4-carboxylic acid [2-(4-methoxy-phenylamino)-phenyl]-amide was used instead of thiophene-3-carboxylic acid [2-(4-methoxy-phenylamino)-phenyl]-amide. MS (M+1) 308.

4-(2-lsothiazol4-yl-benzoimidazol-1-yl)-phenol

4-(2-lsothiazol-4-yl-benzoimidazol-1-yl)-phenol (0.015 g, 0.051,mmol) was prepared in a procedure analogous to that described in example 1 step E except that 2-isothiazol-4-yl-1-(4-methoxy-phenyl)-1H-benzoimidazole was used instead of 1-(4-methoxy-phenyl)-2-thiophen-3-yl-1H-benzoimidazole. MS (M+1)⁺ 294; ¹H NMR (CD₃OD) δ_{H} 9.27 (s, 1H), 8.69 (s, 1H), 7.94-7.96 (d, 1H), 7.73-7.78 (m, 1H), 7.66-7.71 (m, 1H), 7.46-7.52 (m, 3H), 7.10-7.13 (d, 2H).

### Example 7

4-[2-(4-Methyl-isothiazol-5-yl)-benzoimidazol-1-yl)-phenol

4-Methyl-isothiazole-5-carboxylic acid can be prepared according to the procedure of M. P. L. Caton, D. H. Jones, R.Slack and K. R. H. Wooldridge J. Chem. Soc. 1964, 446.

4-Methyl-isothiazole-5-carboxylic acid [2-(4-methoxy-phenylamino)-phenyl]-amide

4-Methyl-isothiazole-5-carboxylic acid [2-(4-methoxy-phenylamino)-phenyl]-amide was prepared in a procedure analogous to that described in example 5 step A except 4-methyl-isothiazole-5-carboxylic acid (0.074 g, 0.522 mmol) was used instead of 3-bromothiophene-2-carboxylic acid.

1-(4-Methoxy-phenyl)-2-(4-methyl-isothiazol-5-yl)-1H-benzoimidazole

1-(4-Methoxy-phenyl)-2-(4-methyl-isothiazol-5-yl)-1H-benzoimidazole was prepared in a procedure analogous to that as described in example 1 step D except that 4-methyl-isothiazole-5-carboxylic acid [2-(4-methoxy-phenylamino)-phenyl]-amide was used instead of thiophene-3-carboxylic acid [2-(4-methoxy-phenylamino)-phenyl]-amide. MS (M+1) 322.

4-[2-(4-Methyl-isothiazol-5-yl)-benzoimidazol-1-yl]-phenol

4-[2-(4-Methyl-isothiazol-5-yl)-benzoimidazol-1-yl]-phenol (0.015 g, 0.0489 mmol) was prepared in a procedure analogous to that as described in example 1 step E except that 1-(4-methoxy-phenyl)-2-(4-methyl-isothiazol-5-yl)-1H-benzoimidazole was used instead of 1-(4-methoxy-phenyl)-2-thiophen-3-yl-1H-benzoimidazole. MS (M+1) 308; ¹H NMR (acetone) δ_{H} 8.56 (s, 1H), 8.32-8.34 (d, 1H), 7.71-7.79 (m, 2H), 7.66-7.68 (d, 2H), 7.56-7.59 (d, 1H), 2.78 (s, 1H).

### Example 8

4-[2-(4-Methyl-[1,2,3]thiadiazol-5-yl)-benzoimidazol-1-yl]-phenol

4-Methyl-[1,2,3]thiadiazole-5-carboxylic acid [2-(4-methoxy-phenylamino)-phenyl]-amide

4-Methyl-[1,2,3]thiadiazole-5-carboxylic acid [2-(4-methoxy-phenylamino)-phenyl]-amide was prepared in a procedure analogous to that as described in example 5 step A except that 4-methyl-[1,2,3]thiadiazole-5-carboxylic acid (0.075 g, 0.521 mmol) was used instead of3-bromothiophene-2-carboxylic acid.

1-(4-Methoxy-phenyl)-2-(4-methyl-[1,2,3]thiadiazol-5-yl)-1H-benzoimidazole

1-(4-Methoxy-phenyl)-2-(4-methyl-[1,2,3]thiadiazol-5-yl)-1H-benzoimidazole was prepared in a procedure analogous to that as described in example 1 step D except that 4-methyl-[1,2,3]thiadiazole-5-carboxylic acid [2-(4-methoxy-phenylamino)-phenyl]-amide was used instead of thiophene-3-carboxylic acid [2-(4-methoxy-phenylamino)-phenyl]-amide. MS -(M+1) 323.

4-[2-(4-Methyl-[1,2,3]thiadiazol-5-yl)-benzoimidazol-1-yl]-phenol

4-[2-(4-Methyl-[1,2,3]thiadiazol-5-yl)-benzoimidazol-1-yl]-phenol (0.015 g, 0.0487 mmol) was prepared in a procedure analogous to that as described in example 1 step E except that 1-(4-methoxy-phenyl)-2-(4-methyl-[1,2,3]thiadiazol-5-yl)-1H-benzoimidazole was used instead of 1-(4-methoxy-phenyl)-2-thiophen-3-yl-1H-benzoimidazole. MS (M+1) 309; ¹H NMR (acetone) 8H 8.24-8.26 (d, 1H), 7.69-7.79 (m, 2H), 7.61-7.64 (d, 2H), 7.57-7.60 (d, 1H), 7.17-7.20 (d, 2H), 3.16 (s, 3H).

### Example 9

4-f2-(3-Chloro-thiophen-2-yl)-benzoimidazol-1-yl]-phenol

3-Chloro-thiophene-2-carboxylic acid [2-(4-methoxy-phenylamino)-phenyl]-amide

3-Chloro-thiophene-2-carboxylic acid [2-(4-methoxy-phenylamino)-phenyl]-amide was prepared in a procedure analogous to that as described in example 5 step A except that 3-chloro-thiophene-2-carboxylic acid (0.094 g, 0.052 mmol) was used instead of 3-bromothiophene-2-carboxylic acid.

2-(3-Chloro-thiophen-2-yl)-1-(4-methoxy-phenyl)-1H-benzoimidazole

2-(3-Chloro-thiophen-2-yl)-1-(4-methoxy-phenyl)-1H-benzoimidazole was prepared in a procedure analogous to that as described in example 1 step D except that 3-chloro-thiophene-2-carboxylic acid [2-(4-methoxy-phenylamino)-phenyl]-amide was used instead of thiophene-3-carboxylic acid [2-(4-methoxy-phenylamino)-phenyl]-amide. MS (M+1) 341.

4-[2-(3-Chloro-thiophen-2-yl)-benzoimidazol-1-yl]-phenol

4-[2-(3-Chloro-thiophen-2-yl)-benzoimidazol-1-yl]-phenol (0.01 5 g, 0.0459 mmol) was prepared in a procedure analogous to that as described in example 1 step E except that 2-(3-chloro-thiophen-2-yl)-1-(4-methoxy-phenyl)-1H-benzoimidazole was used instead of 1-(4-methoxy-phenyl)-2-thiophen-3-yl-1H-benzoimidazole. MS (M+1) 327; ¹H NMR (acetone) δ_{H} 8.16-8.18 (d, 1H), 8.03-8.05 (d, 1H), 7.59-7.58 (m, 2H), 7.50-7.56 (m, 3H), 7.23-7.25 (d, 1H), 7.15-7.18 (d, 2H).

### Example 10

4-[2-(1-Ethyl-1H-pyrrol-2-yl)-benzoimidazol-1-yl]-phenol

Step A

1H-Pyrrole-2-carboxylic acid [2-(4-methoxy-phenylamino)-phenyl]-amide

To a solution of N-(4-methoxy-phenyl)-benzene-1,2-diamine di-hydrochloride salt (2.14 g, 7.46 mmol), pyrrole-2-carboxylic acid (1.24 g, 11.2 mmol), Et₃N (4.17 ml, 29.8 mmol) and DMAP (cat.) in CH₂Cl₂ (20 ml) was added PPAA (50% solution in EtOAc, 6.7 ml, 11.2 mmol). The reaction was stirred at room temperature overnight, diluted with CH₂Cl₂ (80 ml) and washed with sat. NaHCO₃ (2×50 ml). The combined organics were dried (MgSO₄), filtered and concentrated by vacuum to give 1H-pyrrole-2-carboxylic acid [2-(4-methoxy-phenylamino)-phenyl]-amide which was taken directly into the next step. MS (M+1) 308.

Step B

1-(4-Methoxy-phenyl)-2-(1H-pyrrol-2-yl)-1H-benzoimidazole

A solution of crude 1H-pyrrole-2-carboxylic acid [2-(4-methoxy-phenylamino)-phenyl]-amide in AcOH (20 ml) was heated at 60-65° C. overnight. The reaction was allowed to cool to room temperature and the volatiles were removed by vacuum. The residue was taken up in EtOAc (50 ml) and was washed with 10% K₂CO₃ solution (2×50 ml). The combined aqueous washings were back extracted with CH₂Cl₂/MeOH (10:1, 2×50 ml). The combined organics were dried (MgSO₄), filtered and concentrated by vacuum. The solid was subjected to flash chromatography (SiO₂, biotage, EtOAc:hexanes 1:5 to 1:1) to give 1-(4-methoxy-phenyl)-2-(1H-pyrrol-2-yl)-1H-benzoimidazole as a white solid (1.04 g, 3.59 mmol, 48% over two steps). MS (M+1) 290.

Step C

2-(1-Ethyl-1H-pyrrol-2-yl)-1-(4-methoxy-phenyl)-1H-benzoimidazole

To a solution of KO^{t}Bu (0.020 g, 0.181 mmol) and 18-crown-6 (0.048 g, 0.181 mmol) in of THF (1 ml) was added 1-(4-methoxy-phenyl)-2-(1H-pyrrol-2-yl)-l H-benzoimidazole (0.050 g, 0.172 mmol) as a solution in THF (1 ml). The reaction mixture was stirred at room temperature for 15 minutes. The temperature was decreased to 0° C. and (41 μL, 0.52 mmol) of ethyl iodide was added to the reaction. The mixture was stirred at 0° C. for 30 minutes and then at room temperature for 3 hours. An additional aliquot of ethyl iodide (14 μL, 0.181 mmol) was added and the reaction material was stirred at room temperature for an additional 2 hours. The reaction was quenched with saturated NH₄Cl (1 ml) and diluted with CH₂Cl₂ (20 ml), H₂O (2 ml) and saturated NH₄Cl (10 ml). The layers were separated and the aqueous phase was extracted with CH₂Cl₂ (20 ml). The combined organic extracts were dried (MgSO₄), filtered and concentrated by vacuum. Purification by silica gel flash chromatography, eluting with 6:1 hexanes: ethyl acetate, gave 2-(1-ethyl-1H-pyrrol-2-yl)-i-(4-methoxy-phenyl)-1H-benzoimidazole (0.039 g, 0.123 mmol). ¹H NMR (CDCl₃) δ_{H} 7.78-7.79 (d, 1H), 7.16-7.77 (m, 4H), 7.08-7.10 (d, 1H), 6.99-7.01 (d, 2H), 6.77-6.78 (m, 1H), 5.96-5.98 (m, 1H), 5.75-5.76 (dd,1H), 4.52-4.57 (m, 2H), 3.86 (s, 3H), 1.34-1.38 (t, 3H).

Step D

4-[2-(l1-Ethyl-1H-pyrrol-2-yl)-benzoimidazol-1-yl]-phenol

To a solution of 2-(1,ethyl-1H-pyrrol-2-yl)-1-(4-methoxy-phenyl)-1H-benzoimidazole (0.039 g, mmol, 0.123 mmol) CH₂Cl₂ (2 ml) cooled to −78° C. under an atmosphere of nitrogen was added BBr₃ as a 1.0 M solution (0.379 ml, 0.379 mmol). The reaction was stirred overnight while slowly warming to room temperature. Methanol (2 ml) was added to the mixture and it was diluted CH₂Cl₂ (20 ml). The mixture was washed with saturated sodium bicarbonate solution. The aqueous layer was extracted with CH₂Cl₂ (15 ml) and the combined organic extracts were dried (MgSO₄), filtered and concentrated by vacuum. Purification by silica gel flash chromatography (SiO₂, eluting with 2:1 hexane:ethyl acetate) gave 4-[2-(1-ethyl-1H-pyrrol-2-yl)-benzoimidazol-1-yl]-phenol. MS (M+1) 304; ¹H NMR(CDCl₃) δ_{H} 8.78 (s, 1H), 7.78-7.80 (d, 1H), 7.26-7.30 (t, 1H), 7.20-7.24 (t, 1H), 7.14-7.16 (d, 1H), 7.03-7.07 (m, 2H), 6.82-6.86 (m, 2H), 6.73-6.74 (t, 1H), 5.97-5.99 (m, 1H), 5.86-5.88 (m, 1H), 4.34-4.40 (m, 2H), 1.25-1.29 (t, 3H).

### Example 11

4-(2-Furan-3-yl-benzoimidazol-1-yl)-phenol

Step A

Furan-3-carboxylic acid [2-(4-methoxy-phenylamino)-phenyl]-amide

Furan-3-carboxylic acid [2-(4-methoxy-phenylamino)-phenyl]-amide was prepared in a procedure analogous to that as described in example 5 step A. except that furan-3-carboxylic acid (0.076, 0.069 mmol) was used instead of 3-bromothiophene-2-carboxylic acid. MS (M+1) 309.

Step B

2-Furan-3-yl-1-(4-methoxy-phenyl)-1H-benzoimidazole

2-Furan-3-yl-1-(4-methoxy-phenyl)-1H-benzoimidazole was prepared in a procedure analogous to that as described in example 1 step D except that furan-3-carboxylic acid [2-(4-methoxy-phenylamino)-phenyl]-amide was used instead of thiophene-3-carboxylic acid [2-(4-methoxy-phenylamino)-phenyl]-amide. MS (M+1) 291.

Step C

4-(2-Furan-3-yl-benzoimidazol-1-yl)-phenol

4-(2-Furan-3-yl-benzoimidazol-1-yl)-phenol 0.021 g, 0.0719 mmol) was prepared in a manner analogous to that as described in example 1 step E except that 2-furan-3-yl-1-(4-methoxy-phenyl)-1H-benzoimidazole was used instead 1-(4-methoxy-phenyl)-2-thiophen-3-yl-1H-benzoimidazole and the reaction temperature was 0° C. rather than −78° C. and the crude residue was triturated with acetone, filtered and washed with Et₂O. MS (M+1) 277; ¹H NMR (d₆-dmso) δ_{H} 10.05 (s, 1H), 7.65-7.69 (m, 2H), 7.14-7.30 (m, 5H), 6.93-7.01 (m, 3H), 6.55-6.56 (m,1H).

### Example 12

4-[2-(3-Methyl-furan-2-yl)-benzoimidazol-1-yl]-phenol

3-Methyl-furan-2-carboxylic acid [2-(4-methoxy-phenylamino)-phenyl]-amide

3-Methyl-furan-2-carboxylic acid [2-(4-methoxy-phenylamino)-phenyl]-amide was prepared in a procedure analogous that as described in example 5 step A except that 3-methyl-furan-2-carboxylic (0.086 g, 0.69 mmol) acid was used instead of 3-bromothiophene-2-carboxylic acid. MS (M+1) 323.

1-(4-Methoxy-phenyl)-2-(3-methyl-furan-2-yl)-1H-benzoimidazole

1-(4-Methoxy-phenyl)-2-(3-methyl-furan-2-yl)-1H-benzoimidazole was prepared in a procedure analogous to that as described in example 1 step D except that 3-methyl-furan-2-carboxylic acid [2-(4-methoxy-phenylamino)-phenyl]-amide was used instead of thiophene-3-carboxylic acid [2-(4-methoxy-phenylamino)-phenyl]-amide. MS (M+1) 305.

4-[2-(3-Methyl-furan-2-yl)-benzoimidazol-1-yl]-phenol

4-[2-(3-Methyl furan-2-yl)-benzoimidazol-1-yl]-phenol was prepared in a procedure analogous to that as described in example 1 step E, except that 1-(4-methoxy-phenyl)-2-(3-methyl-furan-2-yl)-1H-benzoimidazole (0.052 g, 0.133 mmol) was used instead of 1-(4-methoxy-phenyl)-2-thiophen-3-yl-1H-benzoimidazole, the reaction temperature was 0° C. rather than −78° C. was used and the product was purified by trituration with 10% acetone in Et₂O, filtered and washed with diethyl ether and hexanes. MS (M+)⁺290; ¹H NMR (d₆-dmso) δ_{H} 9.82 (s, 1H), 7.68-7.70 (d, 1H), 7.49 (s, 1H), 7.14-7.25 (m, 4H), 7.05-7.07 (d, 1H), 6.83-6.87 (d, 2H), 6.46 (d, 1H), 2.27 (s, 3H).

### Example 13

4-(2-Furan-2-yl-benzoimidazol-1-yl)-phenol

Furan-2-carboxylic acid [2-(4-methoxy-phenylamino)-phenyl]-amide

Furan-2-carboxylic acid [2-(4-methoxy-phenylamino)-phenyl]-amide was prepared in a procedure analogous to that as described in example 5 step A except that furan-2-carboxylic acid (0.078 g, 0.685 mmol) was used instead of 3-bromothiophene-2-carboxylic acid.

2-Furan-2-yl-1-(4-methoxy-phenyl)-1H-benzoimidazole

2-Furan-2-yl-1-(4-methoxy-phenyl)-1H-benzoimidazole was prepared in a procedure analogous to that described in example 1 step D except that furan-2-carboxylic acid [2-(4-methoxy-phenylamino)-phenyl]-amide was used instead of thiophene-3-carboxylic acid [2-(4-methoxy-phenylamino)-phenyl]-amide.

4-(2-Furan-2-yl-benzoimidazol-1-yl)-phenol

4-(2-Furan-2-yl-benzoimidazol-1-yl)-phenol was prepared in a-procedure analogous to example 1 step E except that 2-furan-2-yl-1-(4-methoxy-phenyl)-1H-benzoimidazole (0.016 g, 0.0580 mmol) was used instead of 1-(4-methoxy-phenyl)-2-thiophen-3-yl-1H-benzoimidazole, a reaction temperature of 0° C. was used and the product was purified by preparatory TLC (SiO₂ EtOAc: hexanes 1:1) and trituration with Et₂O. MS (MH)⁺277; ¹H NMR (d₆-dmso) δ_{H} 10.01 (s, 1H), 7.79-7.80 (d, 1H), 7.68-7.70 (d, 1H), 7.19-7.28 (m, 4H), 7.01-7.03 (d, 1H), 6.93-9.97 (d, 2H), 6.52-6.53 (d, 2H), 6.16-6.17 (d, 1H).

### Example 14

4-[2-(3-Ethyl-isoxazol-4-yl)-benzoimidazol-1-yl]-phenol

Step A

3-Ethyl-isoxazole4-carboxylic acid ethyl ester

A solution of ethyl trans-3-(1-pyrrolidino) acrylate (1.04 g, 6.16 mmol), 1-nitropropane (0.604 g, 0.6 ml, 6.78 mmol), phenylisocyanate (1.28 g, 1.2 ml, 10.8 mmol), and triethylamine (0.094 g, 0.13 ml, 0.924 mmol) in toluene (15 ml) were stirred at room temperature for 2 hours then heated at 60° C. overnight. The reaction was allowed to cool to room temperature, the precipitate was filtered off and washed with toluene (2×10 ml). The combined washings and filtrate were concentrated by vacuum and the residue was purified by flash chromatography (biotage, SiO₂, 10% EtOAc/hexanes) to give 3-ethyl-isoxazole-4-carboxylic acid ethyl ester as a colorless oil which solidified on standing (0.817 g, 4.83 mmol, 71%). ¹H NMR 400 MHz (CDCl₃) δ_{H} 8.80 (1H, s), 4.29 (2H, q J 7.0 Hz), 2.91 (2H, q 7.5 Hz) and 1.34-1.26 (6H overlapping m).

Step B

3-Ethyl-isoxazole-4-carboxylic acid

To a solution of 3-ethyl-isoxazole-4-carboxylic acid ethyl ester (0.538 g, 3.47 mmol) in MeOH/THF (1:1, 6 ml), was added 5N NaOH (2 ml). The reaction mixture was stirred at room temperature overnight. The mixture was then acidified to pH=3 with 6N HCl and diluted with water (15 ml). The aqueous mixture was extracted with EtOAc (3×30 ml), the organic extracts were combined and washed with brine (1×20 ml), dried (MgSO₄), filtered and concentrated by vacuum to give 3-ethyl-isoxazole4-carboxylic acid as an oil (0.382 g, 2.71 mmol, 78%). ¹H NMR 400 MHz (CDCl₃) δ_{H} 8.92 (1H, s), 2.93 (2H, q J 7.5 Hz) and 1.30 (3H, t J 7.5 Hz).

Step C

3-Ethyl-isoxazole-4-carboxylic acid [2-(4-methoxy-phenylamino)-phenyl]-amide

To N-(4-methoxy-phenyl)-benzene-1,2-diamine di-hydrochloride salt (0.324 g, 1.13mmol), 3-ethyl-isoxazole4-carboxylic acid (0.191 g, 1.35 mmol), Et3N (1.6 ml, 11.3 mmol) and DMAP (cat.) in CH₂Cl₂ (4 ml) was added PPM as a 50% solution in EtOAc (2 ml, 1.7 mmol). The reaction was stirred at room temperature overnight, diluted with EtOAc (35 ml) and washed with sat. NaHCO₃ (2×15 ml) and brine (1×15 ml), dried (MgSO₄), filtered and concentrated by vacuum to give 3-ethyl-isoxazole-4-carboxylic acid [2-(4-methoxy-phenylamino)-phenyl]-amide. The material was used without purification in the next step. ¹H NMR 400 MHz (CDCl₃) δ_{H} 8.39 (1H, s), 8.13 (1H, s), 8.03 (1H, s) 7.18−7.10.(3H, m), 6.85−6.56 (5H, m), 3.72 (3H, s), 2.85 (2H, q J 7.5 Hz) and 1.25 (3H, t J 7.5Hz).

Step D

2-(3-Ethyl-isoxazol-4-yl)-1-(4-methoxy-phenyl)-1H-benzoimidazole

A mixture of 3-ethyl-isoxazole-4-carboxylic acid [2-(4-methoxy-phenylamino)-phenyl]-amide (0.379 g, 1.12 mmol) in glacial AcOH (15 ml) was heated overnight at 80° C. The reaction was allowed to cool to room temperature and diluted with heptane (40 ml) and concentrated by vacuum. The residue was taken up in heptane and concentrated again by vacuum. The residue was taken up EtOAc (50 ml) and washed with sat. NaHCO₃ (2×20 ml) and brine (1×20 ml), dried (MgSO₄), filtered and concentrated by vacuum to give 2-(3-ethyl-isoxazol-4-yl)-1-(4-methoxy-phenyl)-1H-benzoimidazole as a yellow oil (0.354 g, 1.11 mmol, 98% yield over two steps). ¹H NMR 400 MHz (CDCl₃) δ_{H} 7.90−7.88 (2H, m), 7.37−7.06 (7H, m), 3.91 (3H,l s), 3.08 (2H, q J 7.5 Hz) and 1.31 (3H, t J 7.5 Hz). MS 320 (M+1).

Step E

4-[2-(3-Ethyl-isoxazol-4-yl)-benzoimidazol-1-yl]-phenol

To a solution of 2-(3-ethyl-isoxazol-4-yl)-1-(4-methoxy-phenyl)-1H-benzoimidazole (0.350 g, 1.10 mmol) in CH₂Cl₂ (4 ml) cooled to −78° C. was added BBr₃ as a 1.0 M, solution in CH₂Cl₂ (2.4 ml, 2.4 mmol). The reaction mixture was stirred overnight slowly warming to room temperature. The reaction was carefully quenched by the addition of MeOH (1 ml). Stirring was continued for 15 minutes, upon which the pH was adjusted pH=7 by the addition of sat. NaHCO₃ and the aqueous layer was extracted with EtOAc (3×20 ml) The combined organic extracts were washed with brine (1×20 ml), dried (MgSO₄), filtered and concentrated by vacuum. The residue was subjected to flash chromatography (SiO₂, biotage, gradient elution with 25% EtOAc/hexanes to 50% EtOAc/hexanes) to give 4-[2-(3-ethyl-isoxazol-4-yl)-benzoimidazol-1-yl]-phenol as an off white solid (0.108 g, 0.346 mmol, 32% yield). ¹H NMR 400 MHz (CD₃OD) δ_{H} 8.26 (1H, s), 77.74 (1H, d J 7.0 Hz), 7.35−7.18 (5H, m), 6.98 (2H, m), 2.89 (2H, q J 7.5 Hz) and 1.20 (3H, t J 7.5 Hz). MS 306 (M+1).

### Example 15

4-[2-(3-Cyclopropyl-isoxazol-4-yl)-benzoimidazol-1-yl]-phenol

Cyclopropanecarbaldehyde oxime

Cyclopropanecarbaldehyde oxime was prepared according to the procedure of Wu and Wang J. Org. Chem. 1994, 59, 622.

Step A

3-Cyclopropyl-isoxazole-4-carboxylic acid ethyl ester

To a solution of N-chlorosuccinimde (8.38 g, 62.7 mmol) in CHCl₃ (100 ml) was added pyridine (0.5 ml, 0.489 g, 6.20 mmol) maintaining a temperature of 25-30° C. with a water bath. Cyclopropanecarbaldehyde oxime (5.34 g, 62.7 mmol) was added to the mixture as a solution in CHCl₃ (20 ml) and stirring was continued at room temperature for 10 minutes. A solution of ethyl trans-3-(1-pyrrolidino) acrylate (5.31 g, 31.4 mmol) in CHCl₃ (15 ml) was added and stirring was continued for 5 minutes, upon which triethylamine (6.3 g, 9.0 ml, 62.7 mmol) was added as a solution in CHCl₃ (20 ml). The reaction mixture was stirred at room temperature for 30 minutes and then at a gentle reflux overnight. The mixture was allowed to cool to room temperature, diluted with water (100 ml) and extracted with Et₂O (4×150 ml). The combined Et₂O extracts were washed with 1N HCl (4×100 ml), dried (MgSO₄), filtered and concentrated by vacuum. The resultant oil was subjected to flash chromatography (biotage, SiO₂, 10% EtOAc/hexanes to 25% EtOAc/hexanes) to give 3-cyclopropyl-isoxazole4-carboxylic acid ethyl ester as an oil (0.126 g, 0.696 mmol, 2%). ¹H NMR 400 MHz (CDCl₃) δ_{H} 8.76 (1H, s), 4.30 (2H, q J 7.0 Hz), 2.41 (1H, m) and 1.40−0.86 (4H, m).

Step B

3-Cyclopropyl-isoxazole-4-carboxylic acid

To a solution of 3-cyclopropyl-isoxazole-4-carboxylic acid ethyl ester (0.099 g, 0.546 mmol) in MeOH/THF (1:1, 1 ml) was added 5N NaOH (0.32 ml). The reaction mixture was stirred at room temperature overnight. The mixture was then acidified to pH=3 with 6N HCl and diluted with water (5 ml). The aqueous mixture was extracted with EtOAc (3×15 ml), the organic extracts were combined and washed with brine (1×15 ml), dried (MgSO₄), filtered and concentrated by vacuum to give 3-cyclopropyl-isoxazole-4-carboxylic acid as an oil (0.063 g, 0.412 mmol, 75%). ¹H NMR 400 MHz (CDCl₃) δ_{H} 8.87 (1H, s), 2.41 (1H, m)and 1.07 (4H, m).

Step C

3-Cyclopropyl-isoxazole-4-carboxylic acid [2-(4-methoxy-phenylamino)-phenyl]-amide

To N-(4-methoxy-phenyl)-benzene-1,2-diamine di-hydrochloride salt (0.172 g, 0.597 mmol) 3-cyclopropyl-isoxazole4-carboxylic acid (0.069 g, 0.398 mmol), Et₃N (0.8 ml, 5.97 mmol) and DMAP (cat.) in CH₂Cl₂ (3 ml) was added PPM as a 50% solution in EtOAc (0.36 ml, 0.597 mmol). The reaction was stirred at room temperature overnight, diluted with EtOAc (35 ml) and washed with sat. NaHCO₃ (2×15 ml) and brine (1×15 ml), dried (MgSO₄), filtered and concentrated by vacuum to give 3-cyclopropyl-isoxazole4-carboxylic acid [2-(4-methoxy-phenylamino)-phenyl]-amide. The material was used without purification in the next step. ¹H NMR 400 MHz (CDCl₃) δ_{H} 8.72 (1H, s), 8.63 (1H, s), 8.05 (1H, m), 7.37−6.72 (7H, m), 5.59 (1H, s), 3.72 (3H, s), 1.95 (1H, m) and 1.49−0.86 (4H, m). MS 350 (M+1).

Step D

2-(3-Cyclopropyl-isoxazol4-yl)-1-(4-methoxy-phenyl)-1H-benzoimidazole

To a solution of 3-cyclopropyl-isoxazole4-carboxylic acid [2-(4-methoxy-phenylamino)-phenyl]-amide (0.129 g, 0.369 mmol) in MeOH (15 ml) was added HCl as a 4M solution in dioxane (1.0 ml, 4.0 mmol). The reaction was heated overnight at reflux, allowed to cool to room temperature and concentrated by vacuum. The residue was taken up EtOAc (40 ml) and washed with sat. NaHCO₃ (2×15 ml) and brine (1×15 ml), dried (MgSO₄), filtered and concentrated by vacuum to give 2-(3-Cyclopropyl-isoxazol-4-yl)-1-(4-methoxy-phenyl)-1H-benzoimidazole as a yellow oil which solidified on standing (0.97 g, 80% yield over two steps). ¹H NMR 400 MHz (CDCl₃) δ_{H} 7.88 (1H, s), 7.87 (1H, m), 7.35−7.14 (5H, overlapping m), 7.64 (2H, m), 3.88 (3H, s), 2.61 (1H, m) and 1.02 (4H, m). MS 332 (M+1).

Step E

4-[2-(3-Cyclopropyl-isoxazol4-yl)-benzoimidazol-1-yl]-phenol

To a solution of 2-(3-cyclopropyl-isoxazol4-yl)-1-(4-methoxy-phenyl)-1H-benzoimidazole (0.090 g, 0.272 mmol) in CH₂Cl₂ (1 ml) cooled to -78° C. was added BBr₃ as a 1.0 M solution in CH₂Cl₂ (0.6 ml, 0.6 mmol). The reaction mixture was stirred overnight slowly warming to room temperature. The reaction was carefully quenched by the addition of MeOH (0.5 ml). Stirring was continued for 15 minutes, upon which the pH was adjusted pH=7 by the addition of sat. NaHCO₃ and the aqueous layer was extracted with EtOAc (3×20 ml) The combined organic extracts were washed with brine (1×20 ml), dried (MgSO₄), filtered and concentrated by vacuum. The residue was subjected to flash chromatography (SiO₂, biotage, gradient elution with 25% EtOAc/hexanes to 50% EtOAc/hexanes) to give 4-[2-(3-cyclopropyl-isoxazol-4-yl)-benzoimidazol-1-yl]-phenol as an off white solid (0.069 g, 0.218 mmol, 80% yield). ¹H NMR 400 MHz (CD₃OD) δ_{H} 8.30 (1H, s), 7.72 (1H, m), ;7.34−7.27 (3H, m), 7.19 (2H, m), 6.94 (2H, m), 2.20 (1H, m) and 0.98−0.85 (4H, m). MS 318 (M+1).

### Example 16

4-[2-(3-ethyl-5-methyl-isoxazol-4-yl)-benzoimidazol-1-yl]-phenol

Step A

3-Ethyl-5-methyl-isoxazole-4-carboxylic acid

To 3-ethyl-5-methyl-isoxazole-4-carboxylic acid ethyl ester (2.82 g, 15.4 mmol) in THF (25 ml) and EtOH (20 ml) was added NaOH as a 5N solution in water (25 ml). The reaction was stirred at room temperature overnight, acidified to pH=3 with 6N HCl, diluted with water to a volume of 100 ml and extracted with EtOAc (3×150 ml). The organics were combined, washed with brine (150 ml), dried (MgSO4), filtered and concentrated by vacuum to give 3-ethyl-5-methyl-isoxazole-4-carboxylic acid as a white solid (2.1 g, 13.5 mmol, 88%). ¹H NMR 400 MHz (CDCl₃) δ_{H} 2.90 (2H, q J 7.5 Hz), 2.69 (3H, s) and 1.30 (3H, t J 7.5 Hz).

Step B

3-Ethyl-5-methyl-isoxazole4-carboxylic acid [2-(4-methoxy-phenylamino)-phenyl]-amide

To N-(4-methoxy-phenyl)-benzene-1,2-diamine di-hydrochloride salt (0.292 g, 1.02 mmol) 3-ethyl-5-methyl-isoxazole-4-carboxylic acid (0.189 g, 0.122 mmol), Et₃N (1.4 ml, 10.2 mmol) and DMAP (cat.) in CH₂Cl₂ 4 ml) was added PPM as a 50% solution in EtOAc (0.36 ml, 0.597 mmol). The reaction was stirred at room temperature overnight, diluted with EtOAc (35 ml) and washed with sat. NaHCO₃ (2×15m1) and brine (1×15 ml), dried (MgSO₄), filtered and concentrated by vacuum to give 3-ethyl-5-methyl-isoxazole4-carboxylic acid [2-(4-methoxy-phenylamino)-phenyl]-amide. The material was used without purification in the next step. ¹H NMR 400 MHz (CDCl₃) δ_{H} 8.20 (1H, m), 8.07 (1H, s), 7.22−7.09 (3H, overlapping ; m) 6.77 (2H, m), 6.74 (2H, m), 5.30 (1H, s), 3.71(3H, s), 2.68 (2H, m), 2.46 (3H, s) and 1.19 (3H, m).

Step C

2-(3-Ethyl-5-methyl-isoxazol-4-yl)-l -(4-methoxy-phenyl)-1H-benzoimidazole

A mixture of 3-ethyl-5-methyl-isoxazole4-carboxylic acid [2-(4-methoxy-phenylamino)-phenyl]-amide (0.338 g, 0.962 mmol) in glacial AcOH (15 ml) was heated overnight at 80° C. The reaction was allowed to cool to room temperature and diluted with heptane (40 ml) and concentrated by vacuum. The residue was taken up in heptane and concentrated again by vacuum. The residue was taken up EtOAc (50 ml) and washed with sat. NaHCO₃ (2×20 ml) and brine (1×20 ml), dried (MgSO₄), filtered and concentrated by vacuum to give 2-(3-Ethyl-5-methyl-isoxazol-4-yl)-1-(4-methoxy-phenyl)-1H-benzoimidazole as a yellow oil (0.290 g, 0.875 mmol, 86% yield over two steps). ¹H NMR 400 MHz (CDCl₃) δ_{H} 7.90 (1H, d J 8.0 Hz), 7.40−7.31 (4H, m), 7.15 (2H, m), 6.98 (2H, m), 3.86 (3H, s), 2.48 (2H, q J 7.5 Hz) and 1.08 (3H, t 7.5 Hz). MS 334 (M+1).

StepD

4-[2-(3-Ethyl-5-methyl-isoxazol-4-yl)-benzoimidazol-1-yl]-phenol

To a solution of 2-(3-ethyl-5-methyl-isoxazol-4-yl)-1-(4-methoxy-phenyl)-1H-benzoimidazole (0.280 g, 0.84 mmol) in CH₂Cl₂ (4 ml) cooled to −78° C. was added BBr₃ as a 1.0 M solution in CH₂Cl₂ (1.9 ml, 1.85 mmol). The reaction mixture was stirred overnight, slowly warming to room temperature. The reaction was carefully quenched by the addition of MeOH (1 ml). Stirring was continued for 15 minutes, upon which the pH was adjusted pH=7 by the addition of sat. NaHCO₃ and the aqueous layer was extracted with EtOAc (3×20 ml) The combined organic extracts were washed with brine (1×20 ml), dried (MgSO₄), filtered and concentrated by vacuum. The residue was subjected to flash chromatography (SiO₂, biotage, gradient elution with 25% EtOAc/hexanes to 50% EtOAc/hexanes) to give 4-[2-(3-ethyl-5-methyl-isoxazol-4-yl)-benzoimidazol-1-yl]-phenol as an off white solid (0.108 g, 0.346 mmol, 32% yield). ¹H NMR 400 MHz (CD₃OD) δ_{H} 7.84 (1H, d J 7.0 Hz), 7.56−7.50 (3H, m), 7.26 (2H, m), 6.98 (2H, m), 2.43 (2H, q J 7.5 Hz), 2.30 (3H, s) and 1.05 (3H, t J 7.5 Hz). MS 320 (M+1).

### Example 17

4-[2-(5-Methyl-isoxazol-4-yl)-benzoimidazol-1-yl]-phenol

Step A

5-Methyl-isoxazole-4-carboxylic acid [2-(4-methoxy-phenylamino)-phenyl]-amide

To N-(4-methoxy-phenyl)-benzene-1,2-diamine di-hydrochloride salt (0.942 g, 3.28 mmol), 5-methyl-isoxazole-4-carboxylic acid (0.500 g, 3.93 mmol), Et₃N (4.6 ml, 32.8 mmol) and DMAP. (cat.) in CH₂Cl₂ (10 ml) was added PPAA as a 50% solution in EtOAc (3.0 ml, 4.92 mmol). The reaction was stirred at room temperature overnight, diluted with EtOAc (100 ml) and washed with sat. NaHCO₃ (2×30 ml) and brine (1×30 ml), dried (MgSO₄), filtered and concentrated by vacuum. The resultant oil was subjected to flash chromatography (SiO₂, biotage, 25% EtOAc/hexanes) to give 5-methyl-isoxazole-4-carboxylic acid [2-(4-methoxy-phenylamino)-phenyl]-amide as an oil (0.729 g, 2.26 mmol, 69%). MS 324 (M+1).

Step B

1-(4-Methoxy-phenyl)-2-(5-methyl-isoxazol-4-yl)-1H-benzoimidazole

To mixture of 5-methyl-isoxazole-4-carboxylic acid [2-(4-methoxy-phenylamino)-phenyl]-amide (0.525 g, 1.62 mmol) in glacial AcOH (15 ml) was heated overnight at 80° C. The reaction was allowed to cool to room temperature and diluted with heptane (20 ml) and concentrated by vacuum. The residue was taken up in heptane and concentrated again by vacuum. The residue was taken up EtOAc (100 ml) and washed with sat. NaHCO₃ (2×30 ml) and brine (1×30 ml), dried (MgSO₄), filtered and concentrated by vacuum. The resultant oil was subjected to flash chromatography (SiO₂, biotage, 10% EtOAc/hexanes) to give 1-(4-methoxy-phenyl)-2-(5-methyl-isoxazol-4-yl)-1H-benzoimidazole as an oil (0.127 g, 0.421 mmol, 26%). ¹H NMR 400 MHz (CDCl₃) δ_{H} 7.86 (1H, d J 8.0 Hz), 7.59 (1H, s), 7.34 (1H, m), 7.29 (1H, m), 7.26 (2H, J 8.5 Hz), 7.17 (1H, d J 8.0 Hz), 7.07 (2H, J 8.5 Hz), 3.90 (3H, s) and 2.79 (3H, s). MS 306 (M+1).

Step C

4-[2-(5-Methyl-isoxazol4-yl)-benzoimidazol-1-yl]-phenol

To a solution of 2-(5-methyl-isoxazol-4-yl)-1-(4-methoxy-phenyl)-1H-benzoimidazole (0.110 g, 0.36 mmol) in CH₂Cl₂ (1.5 ml) cooled to −78° C. was added BBr₃ as a 1.0 M solution in CH₂Cl₂ (0.8 ml, 0.80 mmol). The reaction mixture was stirred overnight slowly warming to room temperature. The reaction was carefully quenched by the addition of MeOH (1 ml). Stirring was continued for 15 minutes, upon which the pH was adjusted pH=7 by the addition of sat. NaHCO₃. The mixture was diluted with EtOAc (30 ml) and washed with sat. NaHCO₃ (2×15 ml) and brine (1×15 ml)., dried (MgSO₄), filtered and concentrated by vacuum. The residue was subjected to flash chromatography (SiO₂, biotage, elution 1% MeOH/CH₂Cl₂) to give 4-[2-(5-methyl-isoxazol-4-yl)-benzoimidazol-1-yl]-phenol as an off white solid (0.056 g, 0.192 mmol, 53% yield). ¹H NMR 400 MHz (CD₃OD) δ_{H} 7.75 ( 1H, d J 8.0 Hz), 7.60 (1H, s), 7.30−7.19 ( 3H, m), 7.08 (2H, d J 8.5 Hz), 6.92 (2H, d J 8.5 Hz) and 2.65 (3H, s). MS 292 (M+1).

### Example 18

4-[2-(3-Methyl-isoxazol4-yl)-benzoimidazol-1-yl]-phenol

Step A

3-Methyl-isoxazole-4-carboxylic acid [2-(4-methoxy-phenylamino)-phenyl]-amide

To N-(4-methoxy-phenyl)-benzene-1,2-diamine di-hydrochloride salt (0.942 g, 3.28 mmol) 3-methyl-isoxazole-4-carboxylic acid (0.500 g, 3.93 mmol), Et₃N (4.6 ml, 32.8 mmol) and DIAP (cat.) in CH₂Cl₂ (10 ml) was added PPAA as a 50% solution in EtOAc (3.0 ml, 4.92 mmol). The reaction was stirred at room temperature overnight, diluted with EtOAc (100 ml) and washed with sat. NaHCO₃ (2×30 ml) and brine (1×30 ml), dried (MgSO₄), filtered and concentrated by vacuum. The resultant oil was subjected to flash chromatography (SiO₂, biotage, 25% EtOAc/hexanes) to give 3-methyl-isoxazole-4-carboxylic acid [2-(4-methoxy-phenylamino)-phenyl]-amide as an oil (0.512 g, 1.57 mmol, 48%). ¹H NMR 400 MHz (CD₃OD) δ_{H} 8.91 (1H, s), 7.43 (1H, d J 7.5 Hz), 7.11−7.07 (5H, m), 6.80 (2H, m), 3.72 (3H, s) and 2.44 (3H, s). MS 324 (M+1).

1-(4-Methoxy-phenyl)-2-(3-methyl-isoxazol-4-yl)-1H-benzoimidazole

To mixture of 3-methyl-isoxazole-4-carboxylic acid [2-(4-methoxy-phenylamino)-phenyl]-amide (0.497 g, 1.54 mmol) in glacial AcOH (15 ml) was heated overnight at 80° C. The reaction was allowed to cool to room temperature and diluted with, heptane (20 ml) and concentrated by vacuum. The residue was taken up in heptane and concentrated again by vacuum. The residue was taken up EtOAc (100 ml) and washed with sat. NaHCO₃ (2×40 ml) and brine (1×40 ml), dried (MgSO₄), filtered and concentrated by vacuum. The resultant oil was subjected to flash chromatography (SiO₂, biotage, 20% EtOAc/hexanes) to give 1-(4-methoxy-phenyl)-2-(3-methyl-isoxazol-4-yl)-1H-benzoimidazole as an oil (0.319 g, 0.421 mmol, 68%). ¹H NMR 400 MHz (CDCl₃) δ_{H} 7.87−7.84 (2H, m), 7.36−7.23 (4H, m), 7.06 (1H, m), 3.89 (3H, s) and 2.59 (3H, s). MS 306 (M+1).

4-[2-(3-Methyl-isoxazol4-yl)-benzoimidazol-1-yl]-phenol

To a solution of 2-(3-methyl-isoxazol-4-yl)-1-(4-methoxy-phenyl)-1H-benzoimidazole (0.225 g, 0.737 mmol) in CH₂Cl₂ (2.0 ml) cooled to −78° C. was added BBr₃ as a 1.0 M solution in CH₂Cl₂ (1.5 ml, 1.50 mmol). The reaction mixture was stirred overnight slowly warming to room temperature. The reaction was carefully quenched by the addition of MeOH (1 ml). Stirring was continued for 15 minutes, upon which the pH was adjusted pH=7 by the addition of sat. NaHCO₃. The mixture was diluted with EtOAc (30 ml) and washed with sat. NaHCO₃ (2×15 ml) and brine (1×15 ml), dried (MgSO₄), filtered and concentrated by vacuum. The residue was subjected to flash chromatography (SiO₂, biotage, elution 4% MeOH/CH₂Cl₂) to 4-[2-(3-methyl-isoxazol-4-yl)-benzoimidazol-1-yl]-phenol as an off white solid (0.067 g, 0.230 mmol, 31% yield). ¹H NMR 400 MHz (CDCl₃) δ_{H} 7.84 (1H, s), 7.75 (1H, d J 8.0 Hz), 7.28−7.18 (2H, m), 7.09−7.04 (3H, m), 6.91 (2H, m) and 2.45 (3H, s). MS 292 (M+1).

### Example 19

4-[2-(2-Methyl-thiophen-3-yl)-benzoimidazol-1-yl]-phenol

2-Methyl-thiophene-3-carboxylic acid

2-Methyl-thiophene-3-carboxylic acid was prepared according to the procedure of D. W. Knight, and A. P. Nott J. Chem. Soc. Trans. 1, 1983, 791-794.

Step A

2-Methyl-thiophene-3-carboxylic acid [2-(4-methoxy-phenylamino)-phenyl]-amide

To a solution of N-(4-methoxy-phenyl)-benzene-1,2-diamine di-hydrochloride salt (0.1490 g, 0.519 mmol) and 2-methyl-thiophene-3-carboxylic acid (0.100 g, 0.70 mmol) and Et₃N (0.37 ml, 2.68 mmol) in CH₂Cl₂ 1.5 ml) was added PPAA (50% solution in EtOAc, 0.31 ml, 0.52 mmol) and DMAP (cat. amount). The reaction was stirred at room temperature for overnight, then polymer supported isocyanate beads (Argonaut technologies 1.7 mmol/g loading, 0.200 g) was added. The reaction mixture was stirred for 4 hours, filtered, washing with CH₂Cl₂ (10 ml) and concentrated by vacuum to give. 2-methyl-thiophene-3-carboxylic acid [2-(4-methoxy-phenylamino)-phenyl]-amide. The material was used directly in the next step. MS 339 (M+1).

1-(4-Methoxy-phenyl)-2-(2-methyl-thiophen-3-yl)-1H-benzoimidazole

A solution of 2-methyl-thiophene-3-carboxylic acid [2-(4-methoxy-phenylamino)-phenyl]-amide in AcOH (1.5 ml) was heated at 60° C. overnight. The reaction was allowed to cool to room temperature and quenched with sat. NaHCO₃ (15 ml). The mixture was extracted with CH₂Cl₂ (3×15 ml), the organics were combined, dried (Na₂SO₄), filtered and concentrated by vacuum. The residue was subjected to flash chromatography (SiO₂, biotage, 20% EtOAc/hexanes) to 1-(4-methoxy-phenyl)-2-(2-methyl-thiophen-3-yl)-1H-benzoimidazole (0.087 g, 0.71 mmol, 52% over two steps). ¹H NMR 300 MHz (CDCl₃) δ_{H} 7.84 (1H, dd J 8.0 and 1.0 Hz), 7.31−7.27 (3H, m), 7.15 (2H, d, J 8.0 Hz), 6.97−6.89 (3H, m) and 2.54 (3H, s). MS 321 (M+1).

4-[2-(2-Methyl-thiophen-3-yl)-benzoimidazol-1-yl]-phenol

To a solution of 1-(4-methoxy-phenyl)-2-(2-methyl-thiophen-3-yl)-1H-benzoimidazole (0.087 g, 0.71 mmol) in CH2Cl2 (4 ml) cooled to 0° C. was added BBr₃ as a 1.0M solution in CH₂Cl₂ (1.08 ml, 1.08 mmol). The reaction was stirred for 6 hours warming to room temperature. The reaction was quenched with MeOH (2 ml) and concentrated by vacuum. The residue was subjected to preparative TLC (SiO₂, 35% EtOActhexanes) to give 4-[2-(2-methyl-thiophen-3-yl)-benzoimidazol-1-yl]-phenol (0.029 g, 94.5 μmol, 35%). ¹H NMR 300 MHz (CD₃OD) δ_{H} 7.88 (1H, d J 7.0 Hz),7.62-7.47 (3H, m), 7.30−7.27 (3H, m), 6.97−6.90 (3H, m) and 2.53 (3H, s). MS 307 (M+1).

### Example 20

4-[2-(2-Methyl-fu ran-3-yl)-benzoimidazol-1-yl]-phenol

Step A

2-Methyl-furan-3-carboxylic acid [2-(4-methoxy-phenylamino)-phenyl]-amide

To a solution of N-(4-methoxy-phenyl)-benzene-1,2-diamine di-hydrochloride salt (0.149 g, 0.519 mmol) and 2-methyl-furan-3-carboxylic acid (0.100 g, 0.70 mmol) and Et₃N (0.37 ml, 2.68 mmol) in CH₂Cl₂ 1.5 ml) was added PPAA (50% solution in EtOAc, 0.31 ml, 0.52 mmol) and DMAP (cat. amount). The reaction was stirred at room temperature overnight, then polymer supported isocyanate beads (Argonaut technologies 1.7 mmol/g loading, 0.200 g) was added. The reaction mixture was stirred for 4 hours, filtered, washing with CH₂Cl₂ (10 ml) and concentrated by vacuum to give 2-Methyl-furan-3-carboxylic acid [2-(4-methoxy-phenylamino)-phenyl]-amide. The material was used directly in the next step. MS 323 (M+1).

1-(4-Methoxy-phenyl)-2-(2-methyl-furan-3-yl)-1H-benzoimidazole

A solution of 2-methyl-furan-3-carboxylic acid [2-(4-methoxy-phenylamino)-phenyl]-amide in AcOH (1.5 ml) was heated at 60° C. overnight. The reaction was allowed to cool to room temperature and quenched with sat. NaHCO₃ (15 ml). The mixture was extracted with CH₂Cl₂ (3×15 ml), the organics were combined and concentrated by vacuum. The residue was subjected to preparative TLC (SiO₂, 35% EtOAc/hexanes) to give 1-(4-methoxy-phenyl)-2-(2-methyl-furan-3-yl)-1H-benzoimidazole (0.070 g, 0.23 mmol, 43% over two steps). ¹H NMR 300 MHz (d₆-acetone) δ_{H} 7.73 (1H, dd J 6.0 and 1.0 Hz), 7.47−7.12 (4H, m), 7.40 (2H, d J 9.0 Hz), 7.18 (2H, d J 9.0 Hz), 5.83 (1H, d J 1.5 Hz), 3.94 (3H, s) and 2.64 (3H, s). MS 305 (M+1).

4-[2-(2-Methyl-furan-3-yl)-benzoimidazol-1-yl]-phenol

To a solution of 1-(4-Methoxy-phenyl)-2-(2-methyl-furan-3-yl)-1H-benzoimidazole (0.020 g, 65.7 μmol) in CH₂Cl₂ (4 ml) was added BBr₃ as a 1.0M solution in CH₂Cl₂ (0.26 ml, 0.26 mmol). The reaction was stirred overnight at room temperature. The reaction was quenched with MeOH (2 ml) and concentrated by vacuum. The residue was subjected to preparative TLC (SiO₂, 20% EtOAc/hexanes) to give 4-[2-(2-methyl-furan-3-yl)-benzoimidazol-1-yl]-phenol (0.010 g, 34.5 [mol, 79%). ¹H NMR 300 MHz (d₆-acetone) δ_{H} 7.71 (1H, m), 7.35−7.07 (8H, m) and 2.65 (3H, s). MS 291 (M+1).

### Example 21

4-r2-(2,5-Dimethyl-furan-3-yl)-benzoimidazol-1-yl]-phenol

2,5-Dimethyl-furan-3-carboxylic acid [2-(4-methoxy-phenylamino)-phenyl]-amide

To a solution of N-(4-methoxy-phenyl)-benzene-1,2-diamine di-hydrochloride salt (0.149 g, 0.519 mmol) and 2,5-dimethyl-furan-3-carboxylic acid (0.100 g, 0.70 mmol) and Et₃N (0.37 ml, 2.68 mmol) in CH₂Cl₂ 1.5 ml) was added PPAA (50% solution in EtOAc, 0.31 ml, 0.52 mmol) and DMAP (cat. amount). The reaction was stirred at room temperature for overnight, then polymer supported isocyanate beads (Argonaut technologies 1.7 mmol/g loading, 0.200 g) was added. The reaction mixture was stirred for 4 hours, filtered, washing with CH₂Cl₂ (10 ml) and concentrated by vacuum to give 2,5-dimethyl-furan-3-carboxylic acid [2-(4-methoxy-phenylamino)-phenyl]-amide. The material was used directly in the next step. MS 337 (M+1).

2-(2,5-Dimethyl-furan-3-yl)-1-(4-methoxy-phenyl)-1H-benzoimidazole

A solution of 2,5-dimethyl-furan-3-carboxylic acid [2-(4-methoxy-phenylamino)-phenyl]-amide in AcOH (1.5 ml) was heated at 60° C. overnight. The reaction was allowed to cool to room temperature and quenched with sat. NaHCO₃ (15 ml). The mixture was extracted with CH₂Cl₂ (3×15 ml), the organics were combined and concentrated by vacuum. The residue was subjected to preparative TLC (SiO₂, 35% EtOAc/hexanes) to give 2-(2,5-dimethyl-furan-3-yl)-1-(4-methoxy-phenyl)-1H-benzoimidazole (0.069 g, 0.217 mmol, 41% over two steps). ¹H NMR 300 MHz (d₆-acetone δ_{H} 7.71 (1H, d J 8.0 Hz), 7.40−7.36 (2H, m), 7.30−7.09 (5H, m), 5.46 (1H, s), 3.93 (3H, s), 2.59 (3H, s) and 2.13 (3H, s). MS 319 (M+1).

4-[2-(2,5-Dimethyl-furan-3-yl)-benzoimidazol-1-yl]-phenol

To a solution of 2-(2,5-Dimethyl-furan-3-yl)-1-(4-methoxy-phenyl)-1H-benzoimidazole (0.020 g, 62.8 μmol) in CH₂Cl₂ (4 ml) was added BBr₃ as a 1.0M solution in CH₂Cl₂ (0.26 ml, 0.26 mmol). The reaction was stirred overnight at room temperature. The reaction was quenched with MeOH (2 ml) and concentrated by vacuum. The residue was subjected to preparative TLC (SiO₂, 20% EtOAc/hexanes) to 4-[2-(2,5-Dimethyl-furan-3-yl)-benzoimidazol-1-yl]-phenol (0.015 g, 49.3 μmol, 78%). ¹H NMR 300 MHz (d₆-acetone) δ_{H} 7.69 (1H, dd J 7.5 and 1.0 Hz), 7.30−7.06 (7H, m), 5.65 (1H, s), 2.60 (3H, s) and 2.11 (3H, s). MS 305 (M+1).

### Example 22

4-[2-(1-Propyl-1H-pyrrol-2-yl)-benzoimidazol-1-yl]-phenol

Step A

1-(4-Methoxy-phenyl)-2-(1-propyl-1H-pyrrol-2-yl)-1H-benzoimidazole

To a solution of 1-(4-methoxy-phenyl)-2-(1H-pyrrol-2-yl)-1H-benzoimidazole (0.075 g, 0.25 mmol) and 18-crown-6 (0.206 g, 0.779 mmol) in THF (3.5 ml) cooled to 0° C. was added KHMDS as a 0.5 M solution in toluene (1.6 ml, 0.8 mmol). The reaction mixture was stirred at 0° C. for 1 hour whilst slowly allowing the mixture to warm to room temperature, upon which 1-iodo propane (0.120 ml, 1.30 mmol) was added. The reaction was stirred at room temperature overnight, quenched with sat. NH₄Cl (2 ml), and diluted with CH₂Cl₂ (10 ml). The mixture was washed with sat. NH₄Cl/water (1:1 1×10 ml) and the aqueous washing was extracted with CH₂Cl₂ (1×15 ml). The combined organics were dried (MgSO₄), filtered and concentrated by vacuum to give an oil which was subjected to flash chromatography (SiO₂, biotage, 7:1 hexanes:EtOAc) to give 1-(4-methoxy-phenyl)-2-(1-propyl-1H-pyrrol-2-yl)-1H-benzoimidazole as a colorless oil (0.036 g, 0.109 mmol, 45%). ¹H NMR 400 MHz (CDCl₃) δ_{H} 7.78 (1H, d J 8.0 Hz), 7.28−7.17 (4H, m), 7.08 (1H, m), 6.99 (2H, d J 8.5 Hz), 6.75 (1H, dd J 1.5 and 1.0 Hz), 5.96 (1H, m), 5.76 (1H, m), 4.46 (2H, m), 3.86 (3H, s), 1.71 (2H, m) and 0.84 (3H, t J 7.5 Hz). MS 276 (M+1).

Step B

4-[2-(1-Propyl-1H-pyrrol-2-yl)-benzoimidazol-1-yl]-phenol

To a solution of 1-(4-methoxy-phenyl)-2-(1-propyl-1H-pyrrol-2-yl)-1H-benzoimidazole (0.035 g, 0.106 mmol) in CH₂Cl₂ (2.0 ml) cooled to −78° C. was added BBr₃ as a 1.0 M solution in CH₂Cl₂ (0.32 ml, 0.32 mmol). The reaction mixture was stirred overnight slowly warming to room temperature. The reaction re-cooled to −78° C. and was carefully quenched by the addition of MeOH, (2 ml). The mixture was diluted with CH₂Cl₂ (20 ml) and washed with sat. NaHCO₃ (1×20 ml) and the aqueous layer was back extracted with CH₂Cl₂/MeOH (9:1, 1×20 ml). The organics were combined, dried (MgSO₄), filtered and concentrated by vacuum. The residue was subjected to flash chromatography (SiO₂, biotage, elution 3:2 if hexanes:EtOAc) to give 4-[2-(1-propyl-1H-pyrrol-2-yl)-benzoimidazol-1-yl]-phenol as an oil (0.013 g, 41.0 μmol, 39% yield). ¹H NMR 400 MHz (CD₃OD) δ_{H} 7.67 (1H, m), 7.29−7.20 (2H, m), 7.15−7.07 (3H, m), 6.91−6.83 (3H, m), 5.98 (1H, m), 5.93 (1H, m), 4.21 (2H, q J 7.0Hz), 1.56 (2H, m) and 0.73 (3H, t J 7.5 Hz). MS 318 (M+1).

### Example 23

4-[2-(1-Isopropyl-1H-pyrrol-2-yl)-benzoimidazol-1-yl]-phenol

Step A

4-[2-(1H-Pyrrol-2-yl)-benzoimidazol-1-yl]-phenol

To a solution of 1-(4-methoxy-phenyl)-2-(1H-pyrrol-2-yl)-1H-benzoimidazole (0.644 g, 2.23 mmol) in CH₂Cl₂ (20 ml) cooled to −78° C. was added BBr₃ as a 1.0M solution in CH₂Cl₂ (6.7 ml, 6.7 mmol). The reaction was stirred overnight, slowly warming to room temperature, re-cooled to −78° C. and quenched with MeOH (8 ml). The reaction mixture was diluted with CH₂Cl₂ (60 ml) and MeOH (20 ml) and washed with sat. NaHCO₃ (1×80 ml). The aqueous washing was back extracted with 10% MeOH/CH₂Cl₂ (1×70 ml). The combined organics were dried (MgSO₄) and concentrated by vacuum to give 4-[2-(1H-pyrrol-2-yl)-benzoimidazol-1-yl]-phenol which was used without purification. ¹H NMR 400 MHz (CD₃OD) δ_{H} 7.78 (1H, m) 7.60−7.24 (8H, m), 7.06 (2H, m), 6.40 (1H, m), 6.30 (1H, m) and 5.46 (1H, m).

Step B

1-(4-Benzyloxy-phenyl)-2-(1H-pyrrol-2-yl)-1H-benzoimidazole

To a solution of 4-[2-(1H-pyrrol-2-yl)-benzoimidazol-1-yl]-phenol (0.377 g, 1.37 mmol) and 18-crown-6 (0.398 g, 0.1.51 mmol) in THF (20 ml) added KHMDS as a 0.5 M solution in toluene (3.0 ml, 1.5 mmol). The reaction mixture was stirred room temperature for 1 hour, upon which benzyl bromide (0.245 ml, 2.06 mmol) was added. The reaction was stirred at room temperature for 2 hours, quenched with sat. NH₄Cl (5 ml), and diluted with CH₂Cl₂ (100 ml). The mixture was washed with sat. NH₄Cl (1×25 ml) and the aqueous washing was extracted with CH₂Cl₂ (1×30 ml). The combined organics were dried (MgSO₄), filtered and concentrated by vacuum to give an oil which was subjected to flash chromatography (SiO₂, biotage, 2:1 hexanes:EtOAc) to give 1-(4-benzyloxy-phenyl)-2-(1H-pyrrol-2-yl)-1H-benzoimidazole as a colorless oil (0.365 g, 1.00 mmol, 73%). ¹H NMR 400 MHz (CDCl₃) δ_{H} 10.86 (1H, s), 7.74 (1H, d J 8.0 Hz), 7.51−7.26 (5H, m), 7.25−7.17 (4H, m), 7.04 (1H, d J 8.04 Hz), 6.94 (1H, m), 6.08 (1H, m), 5.67 (1H, m) and 5.18 (2H, s). MS 366 (M+1).

Step C

1-(4-Benzyloxy-phenyl)-2-(1-isopropyl-1H-pyrrol-2-yl)-1H-benzoimidazole

To a solution of 1-(4-benzyloxy-phenyl)-2-(1H-pyrrol-2-yl)-1H-benzoimidazole (0.100 g, 0.273 mmol) and 18-crown-6 (0.144 g, 0.546 mmol) in THF (2 ml) was added KHMDS as a 0;5 M solution in-toluene (1.1 ml, 0.55 mmol). The reaction mixture was stirred at room temperature for 30 minutes, upon which iso-propyl iodide (0.134 ml, 1.37 mmol) was added. The reaction was stirred at room temperature overnight, then further 18-crown-6 (0.145 g, mmol) and KHMDS as a 0.5 M solution in toluene (1.1 ml, 0.55 mmol) was added. The reaction was stirred at room temperature for 60 hours, quenched with sat. NH₄Cl (1 ml), and diluted with CH₂Cl₂ (20 ml). The mixture was washed with sat. NH₄Cl (1×20 ml) and the aqueous washing was extracted with CH₂Cl₂ (1×20 ml). The combined organics were dried (MgSO₄), filtered and concentrated by vacuum to give an oil which was subjected to flash chromatography (SiO2, biotage, 5:1 hexanes:EtOAc) to give 1-(4-benzyloxy-phenyl)-2-(1H-pyrrol-2-yl)-1H-benzoimidazole as a colorless oil (0.030 g, 73.7 μmol, 27%). ¹H NMR 400 MHz (CDCl₃) δ_{H} 7.78 (1H, m), 7.46−7.13 (9H, m), 7.05 (2H, m), 6.90 (1H, m), 6.03 (1H, m), 5.85 (1H, m), 5.49 (1H, septet J 6.5 Hz) and 1.35 (6H, d J 6.5 Hz). MS 408 (M+1).

Step D

4-[2-(1-Isopropyl-1H-pyrrol-2-yl)-benzoimidazol-1-yl]-phenol

To a solution of 1-(4-benzyloxy-phenyl)-2-(1-isopropyl-1H-pyrrol-2-yl)-1H-benzoimidazole (0.028 g, 68.8 μmol) and NH₄HCO₂ (0.092 g, 1.47 mmol) in MeOH (4 ml) was added a catalytic amount of Pd black. The mixture was heated at reflux for 3 hours, diluted with CH₂Cl₂ (10 ml) and filtered through diatomaceous earth. The filtrate was washed with water (1×20 ml) and the aqueous layer was back extracted with CH₂Cl₂ (1×20 ml). The combined organics were dried (MgSO₄), filtered and concentrated. The residue was subjected to flash chromatography (SiO₂, biotage, EtOAc:hexanes 2:3) to give the 4-[2-(1-isopropyl-1H-pyrrol-2-yl)-benzoimidazol-1-yl]-phenol (0.012 g, 37.9 μmol, 55%). ¹H NMR 400 MHz (CD₃OD) δ_{H} 7.68 (1H, m), 7.31−7.19 (3H, m), 7.09 (2H, m), 6.97 (1H, m), 6.05 (2H, m), 4.79 (1H, septet J 6.5 Hz) and 1.24 (6H, d J 6.5 Hz). MS 318 (M+1).

### Example 24

3-Methyl-4-[2-(1-methyl-1H-pyrrol-2-yl)-benzoimidazol-1-yl]-phenol

Step A

(4-Methoxy-2-methyl-phenyl)-(2-nitro-phenyl)-amine

A mixture of 1-fluoro-2-nitrobenzene (5.46 g, 0.0387 mol), 4-methoxy-2-methyl aniline (5.31 g, 0.0387 mol), and potassium carbonate (8.02 g, 0.058 mol) was heated to 150° C. for 72 h. The residue was allowed to cool to room temperature, taken up with water and extracted with ethyl acetate. The combined organic extracts were washed with brine, dried over magnesium sulfate, and concentrated. The crude residue was purified by silica gel flash chromatography, eluting with 33% CH₂Cl₂ in hexane, to give (4-methoxy-2-methyl-phenyl)-(2-nitro-phenyl)-amine (8.65 g, 0.0332 mol, 86%). MS (M+1) 261; ¹H NMR (acetone) δ_{H} 9.21 (s, 1H), 8.13-8.15 (d, 1H), 7.38-7.42 (t, 1H), 7.18-7.20 (d, 1H), 6.93-6.94 (d, 1H), 6.84-6.87 (d, 1H), 6.73-6.77 (t, 1H), 6.61-6.64 (d, 1H), 3.80(s, 3H), 2.18 (s, 3H).

Step B

N-(4-Methoxy-2-methyl-phenyl)-benzene-1,2-diamine di-hydrochloride

To a solution of (4-methoxy-2-methyl-phenyl)-(2-nitro-phenyl)-amine (8.35 g, 0.0323 mol) and palladium black (0.62 g) in of methanol (95 ml) was added 4N hydrochloric acid in 1,4-dioxane (16.2 ml). The solution, kept under 50 p.s.i. H₂, was shaken on a Parr shaker over night. The reaction mixture was filtered through a plug of diatomaceous earth and concentrated by vacuum to give N-(4-methoxy-2-methyl-phenyl)-benzene-1,2-diamine as its bis-hydrochloride salt. (MS (M+1) 229; ¹H NMR (CDCl₃) δ_{H} 7.40 (s, 1H), 7.01-7.03 (t, 1H), 6.88-6.90 (d, 1H), 6.56-6.67 (m, 3H), 6.46-6.48 (d, 1H), 3.74 (s, 3H), 2.01 (s, 3H).

Step C

1-Methyl-1H-pyrrole-2-carboxylic acid [2-(4-methoxy-2-methyl-phenylamino)-phenyl]-amide

To a solution of N-(4-methoxy-2-methyl-phenyl)-benzene-1,2-diamine di-hydrochloride (0.17 g , 0.56 mol) 1-methyl-2-pyrrole-carboxylic acid (0.106 g, 0.85 mmol), triethylamine (0.286 g, 0.39 ml, 2.833 mmol), and a catalytic amount of 4-dimethylaminopyridine in CH₂Cl₂ was added 50% 1-propanephosphonic acid cyclic anhydride in ethyl acetate (0.340 mL, 1.13 mmol)) and the mixture was stirred over night at room temperature. Isocyanate scavenger beads (Argonaut Technologies) were added to the reaction mixture to remove excess N-(4-methoxy-2-methyl-phenyl)-benzene-1,2-diamine and the suspension was stirred for several hours. The beads were removed by filtration, and the filtrate was concentrated by vacuum to give 1-methyl-1H-pyrrole-2-carboxylic acid [2-(4-methoxy-2-methyl-phenylamino)-phenyl]-amide which was taken on directly into the next. MS (M+1)⁺ 336.

Step D

1-(4-Methoxy-2-methyl-phenyl)-2-(1-methyl-1H-pyrrol-2-yl)-1H-benzoimidazole

To the crude 1-methyl-1H-pyrrole-2-carboxylic acid [2-(4-methoxy-2-methyl-phenylamino)-phenyl]-amide was added glacial acetic acid (2 mL). The reaction mixture was stirred at 60° C. for two days and then at 80° C. over night. The reaction mixture was neutralized with saturated sodium bicarbonate and extracted with CH₂Cl₂. Combined organic extracts were dried over MgSO₄ and concentrated. The crude reside was purified by silica gel chromatography (EtOAc 1:8 hexanes), to give 1-(4-methoxy-2-methyl-phenyl)-2-(1-methyl-1H-pyrrol-2-yl)-1H-benzoimidazole (0.040 g, 1.26 mmol). MS (M+1) 318; ¹H NMR (acetone) SH 7.69-7.70 (d, 1H), 7.15-7.28 (m, 3H), 7.04-7.05 (d, 1H), 6.98-7.01 (m, 1H), 6.87-6.91 (m, 2H), 5.89-5.91 (m, 1H), 5.63-5.65 (m, 1H), 4.15 (s, 3H), 3.90 (s, 3H), 1.83 (s, 3H).

Step E

3-Methyl4-[2-(1-methyl-1H-pyrrol-2-yl)-benzoimidazol-1-yl]-phenol

To a solution of 1-(4-Methoxy-2-methyl-phenyl)-2-(i -methyl-1H-pyrrol-2-yl)-1H-benzoimidazole (0.040 g, 1.26 mmol in CH₂ Cl₂ (1 ml) cooled to −78° C. was added BBr₃ as a 1.0M solution in CH₂Cl₂ (0.40 ml, 0.40 mmol). The reaction mixture was stirred overnight, slowly warming to room temperature. The mixture was re-cooled to −78° C. and quenched by the addition of MeOH (0.5 ml). The mixture was diluted with CH₂Cl₂ (20 ml) and neutralized with sat. NaHCO₃. The layers were separated and the aqueous layer was extracted with CH₂Cl₂. The combined organics were dried (MgSO₄), filtered and concentrated by vacuum to give 3-methyl-4-[2-(1-methyl-1H-pyrrol-2-yl)-benzoimidazol-1-yl]-phenol (0.034 g, 0.112 mmol). MS (M+1) 304; ¹H NMR (CD₃OD) δ_{H} 7.62-7.70 (d, 1H), 7.18-7.28 (m, 2H),7.07-7.09 (d, 1H), 6.93-6.95 (d, 1H), 6.75-6.82 (m, 3H), 5.92-5.94 (m, 1H), 5.76-5.77 (m, 1H), 3.95 (s, 3H), 1.71 (s, 3H).

### Example 25

4-[2-(3,5-Dimethyl-isoxazol4-yl)-benzoimidazol-1-yl]-3-methyl-phenol

Step A

3,5-Dimethyl-isoxazole4-carboxylic acid [2-(4-methoxy-2-methyl-phenylamino)-phenyl]-amide

To a solution of N-(4-methoxy-2-methyl-phenyl)-benzene-1,2-diamine (0.200 g, 0.877 mmol), 3,5-dimethylisoxazole4-carboxylic acid (0.186 9, 1.3155 mmol), triethylamine ( 0.613 mL, 4.385 mmol), and a catalytic amount of 4-Dimethylaminopyridine in CH₂Cl₂ (² ml), was added 50%1-propanephosphonic acid cyclic anhydride added (1.05 mL, 1.754 mmol) in ethyl acetate and stirred overnight at room temperature. The reaction material was diluted with CH₂Cl₂, washed with saturated sodium bicarbonate and extracted into CH₂Cl₂. The combined organic material was dried (MgSO₄), filtered, and concentrated, giving 3,5-dimethyl-isoxazole-4-carboxylic acid [2-(4-methoxy-2-methyl-phenylamino)-phenyl]-amide. MS (M+1) 352.

Step B

2-(3,5-Dimethyl-isoxazol-4-yl)-1-(4-methoxy-2-methyl-phenyl)-1H-benzoimidazole

To a solution of 3,5-dimethyl-isoxazole-4-carboxylic acid [2-(4-methoxy-2-methyl-phenylamino)-phenyl]-amide in anhydrous methanol (7 ml), was added HCl as a 4.0 M solution in 1,4-dioxane (1.1 ml, 4.385 mmol) and the solution was stirred at 60° C. overnight. Additional HCl in dioxane(1 mL, 4.0 mmol) and MeOH (1 mL) were added and the solution was stirred at 60° C. overnight again. The reaction material was neutralized with saturated sodium bicarbonate and extracted into CH₂Cl₂. The organic extract was dried (MgSO₄), filtered and concentrated. Purification by Biotage flash chromatography (SiO₂), eluting with CH₂Cl₂, gave 2-(3,5-Dimethyl-isoxazol4-yl)-1-(4-methoxy-2-methyl-phenyl)-1H-benzoimidazole (0.151 g, 0.452 mmol, 52% yield over two steps). MS (M+1) 334; ¹H NMR (CDCl₃) δH 7.82-7.84 (d, 1H), 7.28-7.32 (t, 1H), 7.21-7.25 (t, 1H), 7.02-7.05 (m, 2H), 6.76-7.81 (m, 2H), 3.79 (s, 3H), 2.16 (s, 3H), 2.08 (s, 3H), 1.87 (s, 3H).

Step C

4-[2-(3,5-Dimethyl-isoxazol4-yl)-benzoimidazol-1-yl]-3-methyl-phenol

To a solution of 2-(3,5-Dimethyl-isoxazol4-yl)-1-(4-methoxy-2-methyl-phenyl)-1H-benzoimidazole (0.143 g, 0.447 mmol) in CH₂Cl₂ (2 ml) cooled to -78° C. under an atmosphere of nitrogen was added BBr₃ as a 1.0 M solution in CH₂Cl₂ (1.34 mL, 1.34 mmol) and the reaction was stirred as the solution warmed to room temperature overnight. The mixture was re-cooled to −78° C. and the reaction was quenched with methanol (0.3 ml). The mixture was allowed to warm to room temperature upon which it was neutralized with saturated sodium bicarbonate and extracted with CH₂Cl₂. The combined organic material was dried over MgSO₄, filtered, and concentrated, giving 4-[2-(3,5-dimethyl-isoxazol-4-yl)-benzoimidazol-1-yl]-3-methyl-pheno (0.2725 g, 0.854 mmol). MS (M+1)⁺ 320; ¹H NMR (CD₃OD) δ_{H} 7.98-8.00 (d, 1H), 7.71-7.75 (t, 1H), 7.65-7.69 (t, 1H), 7.42-7.44 (d, 1H), 7.28-7.30 (d, 1H), 6.90-6.91 (s, 1H), 6.81-6.84 (dd, 1H), 2.37 (s, 3H), 2.12 (s, 3H), 1.98 (s, 3H).

### Example 26

4-[2-(3-Methyl-thiophen-2-yl)-benzoimidazol-1-yl]-phenol

Step A

3-Methyl-thiophene-2-carboxylic acid [2-(4-methoxy-phenylamino)-phenyl]-amide

To a solution of N-(4-methoxy-phenyl)-benzene-1,2-diamine di-hydrochloride (0.10 g, 0.329 mmol), 3-methyl-thiophene-2-carboxylic acid (0.074 g, 0.523 mmol), Et₃N (0.176 g, 0.24 ml, 1.743 mmol) and DMAP (cat.) in 1,2 dichloroethane (2 ml) was added PPM (0.209 m, 0.697 mmol) as a 50% solution in EtOAc. The reaction was stirred overnight at room temperature and polymer supported isocyanate beads (Argonaut Technologies) (0.200 g, 1.7 mmol/g loading). The reaction was stirred for 4 hours, then the beads were removed by filtration and the mixture was concentrated under stream of N₂ to give 3-methyl-thiophene-2-carboxylic acid [2-(4-methoxy-phenylamino)-phenyl]-amide, which was used without further purification. MS (M+1) 339.

Step B

1-(4-Methoxy-phenyl)-2-(3-methyl-thiophen-2-yl)-1H-benzoimidazole

1-(4-Methoxy-phenyl)-2-(3-methyl-thiophen-2-yl)-1H-benzoimidazole was prepared in a procedure analogous to that as described in example 1step D except that 3-methyl-thiophene-2-carboxylic acid [2-(4-methoxy-phenylamino)-phenyl]-amide was used instead of thiophene-3-carboxylic acid [2-(4-methoxy-phenylamino)-phenyl]-amide. MS (M+1) 321.

Step C

4-[2-(3-Methyl-thiophen-2-yl)-benzoimidazol-1-yl]-phenol

4-[2-(3-Methyl-thiophen-2-yl)-benzoimidazol-1-yl]-phenol was prepared in a procedure analogous to that described in example 1 step E except that 1-(4-methoxy-phenyl)-2-(3-methyl-thiophen-2-yl)-1H-benzoimidazole was used instead of 1-(4-methoxy-phenyl)-2-thiophen-3-yl-1H-benzoimidazole and the product was purified by reverse phase HPLC. NMR (CD₃OD) δ_{H} (1H, m), 7.45 (1H, d, J 5.0 Hz), 7.36−7.27 (3H, m), 7.05 (2H, m), 6.90 (1H, J 5.0 Hz), 6.80 (2H, m) and 2.19 (3H, s).

### Example 27

4-(2-Isothiazol-5-yl-benzoimidazol-1-yl)-phenol

Isothiazole-5-carboxylic acid can be prepared according to the procedure of M. P. L. Caton, D. H. Jones, R.Slack and K. R. H. Wooldridge J. Chem. Soc. 1964, 446.

Step A

Isothiazole-5-carboxylic acid [2-(4-methoxy-phenylamino)-phenyl]-amide

Isothiazole-5-carboxylic acid [2-(4-methoxy-phenylamino)-phenyl]-amide was prepared in a procedure analogous to that as described in example 5 step A except that isothiazole-5-carboxylic acid (0.067 g, 0.521 mmol) was used instead of 3-bromothiophene-2-carboxylic acid.

Step B

2-lsothiazol-5-yl-1-(4-methoxy-phenyl)-1H-benzoimidazole

2-Isothiazol-5-yl-1-(4-methoxy-phenyl)-1H-benzoimidazole was prepared in a procedure analogous to that as described in example 1 step D except thatisothiazole-5-carboxylic acid [2-(4-methoxy-phenylamino)-phenyl]-amide was used instead of thiophene-3-carboxylic acid [2-(4-methoxy-phenylamino)-phenyl]-amide. MS (M+1) 308.

Step C

4-(2-Isothiazol-5-yl-benzoimidazol-1-yl)-phenol

4-(2-lsothiazol-5-yl-benzoimidazol-1-yl)-phenol was prepared in a procedure analogous to that as described in example 1 step E except that 2-isothiazol-5-yl-1-(4-methoxy-phenyl)-1H-benzoimidazole was used instead of 1-(4-methoxy-phenyl)-2-thiophen-3-yl-1H-benzoimidazole. NMR (CD₃OD) δ_{H.} 8.65 (1H, d, J 2.0 Hz), 8.05 (1H, d, J 2.0 Hz), 7.93 (1H, d, J 8.5 Hz), 7.71 (1H, m), 7.64 (1H, m), 7.48 (2H, m), 7.41 (1H, d, J 8.5Hz) and 7.13 (2H, m). MS (M+1) 294.

### Example 28

4-(2-Phenyl-benzoimidazol-1-yl)-phenol

Step A

1-(4-Methoxy-phenyl)-2-phenyl-1H-benzoimidazole

To a suspension of 2-phenyl benzimidazole (0.300 g, 1.55 mmol), p-methoxy phenylboronic acid (0.476 g, 3.10 mmol), copper,(II) acetate,(0.421 g, 2.32 mmol) and molecular sieves (ca. 2 g) in CH₂Cl₂ (10 ml) was added pyridine (0.244 g, 0.250 ml, 3.10 mmol). The reaction mixture was stirred overnight at room temperature, diluted with EtOAc (100 ml) and filtered through diatomaceous earth. The organic solution was washed with 10% K₂CO₃ (2×60 ml), dried (MgSO₄), filtered and concentrated by vacuum. The residue was subjected to flash chromatography (SiO₂ 2:1 hexanes:EtOAc) to give 1-(4-methoxy-phenyl)-2-phenyl-1H-benzoimidazole as a colorless oil (0.059 g, 0.195 mmol, 13%). MS (M+1)+301; ¹H NMR (CD₃OD) δ_{H} 7.70-7.73 (m, 1H), 7.48-7.50 (m, 2H), 7.14-7.35 (m, 8H), 6.99-7.01 (m, 2H), 3.80 (s, 3H),

StepB

4-(2-Phenyl-benzoimidazol-1-yl)-phenol

4-(2-Phenyl-benzoimidazol-1-yl)-phenol was prepared from 1-(4-methoxy-phenyl)-2-phenyl-1H-benzoimidazole in a procedure analogous to that as described in example 1 step E. MS (MH)⁺ 287; ¹H NMR (CD₃OD) δ_{H} 7.71 (d, 1H), 7.52-7.54 (m, 2H), 7.24-7.40 (m, 5H), 7.18-7.20 (m, 1H), 7.12 (d, 2H), 6.86-6.90 (m, 2H).

### Example 29

4-[2-(4-hydroxyl-phenyl)-benzoimidazol-1-yl]-phenol

Step A

4-Methoxy-N-[2-(4-methoxy-phenylamino)-phenyl]-benzamide

To a solution of (4-methoxy-phenyl)-(2-nitro-phenyl)-amine (0.17 g, 0.58 mmol) THF (5 ml) was added of triethylamine (0.49 mL, 3.48 mmol). The solution was cooled to 0° C. and 0.098 g (0.58 mmol) of p-anisoyl chloride and a catalytic amount of 4-dimethylaminopyridine was added to the reaction mixture. The mixture was stirred for 10 min, the ice bath was removed and the mixture was allowed to warm to room temperature over 17 h. The reaction mixture was diluted with EtOAc (20 mL) and washed with saturated aqueous bicarbonate (2×20mL) and water (1×10 mL). The organic phase was dried (MgSO₄) and concentrated by vacuum to give of 4-methoxy-N-[2-(4-methoxy-phenylamino)-phenyl]-benzamide, (0.17, mmol). MS (M+1)⁺ 349; ¹H NMR (CDCl₃) δ_{H} 8.28 (bs, 1H), 8.00-8.03 (m, 1H), 7.65-7.69 (m, 2H), 7.08-7.16 (m, 3H), 6.86-6.89 (m, 2H), 6.78-6.83 (m, 3H), 3.82 (s, 3H), 3.74 (s, 3H).

StepB

1,2-Bis-(4-methoxy-phenyl)-1H-benzoimidazole

A solution of 4-methoxy-N-[2-(4-methoxy-phenylamino)-phenyl]-benzamide (0.17 g, 0.49 mmol) was in glacial acetic acid (5 ml) was heated at 80° C. for 17 h. The reaction mixture was diluted in ethyl acetate and the mixture was washed with saturated aqueous bicarbonate (4×20mL). The aqueous phase was extracted with ethyl acetate and the combined organic extracts were dried (MgSO₄) and concentrated by vacuum to give 1,2-bis-(4-methoxy-phenyl)-1H-benzoimidazole (0.16 g, mmol). MS (MH)⁺ 331; ¹H NMR (CDCl₃) δ_{H} 7.84 (d, 1H), 7.52 (d, 2H), 7.13-7.31 (m, 5H), 6.96-7.00 (m, 2H), 6.78-6.82 (m, 2H), 3.85 (s, 3H), 3.77 (s, 3H).

StepC

4-[2-(4-hydroxyl-phenyl)-benzoimidazol-1-yl]-phenol

4-[2-(4-hydroxyl-phenyl)-benzoimidazol-1-yl]-phenol (0.108 g, mmol) was prepared from 1,2-bis-(4-methoxy-phenyl)-1H-benzoimidazole in a procedure analogous to that as described in example 1 step E except that the crude residue was purified by silica gel chromatography eluting with EtOAc:Hexane, (3:2). MS (M+1)⁺ 303; ¹H NMR (CD₃OD) δ_{H} 7.67 (d, 1H), 7.36-7.40 (m, 2H), 7.24-7.36 (m, 2H), 7.13-7.19 (m, 3H), 6.90-6.93 (m, 2H), 6.72-6.75 (m, 2H).

General Procedure

Step 1

To a solution of acid (1.5 eq.), Et₃N (5.0 eq.), DMAP and N-(4-methoxy-phenyl)-benzene-1,2-diamine di-hydrochloride (1.0 eq) in CH₂Cl₂ (1.5 ml) was added PPAA (2.0 eq.). The reaction was stirred overnight at room temperature. Unreacted N-(4-methoxy-phenyl)-benzene-1,2-diamine was scavenged with polymer supported isocyanate (Argonaut Technologies). The polymer beads are removed via filtration and the volatiles are removed under a stream of N₂.

Step 2

The residue is then taken up in AcOH (3 ml) and heated at 80° C. overnight. The AcOH is removed by vacuum and the residue is taken up in CH₂Cl₂ (5 ml) and washed with sat. NaHCO₃ (until pH of aqueous >7). The aqueous layer is further extracted with CH₂Cl₂ (1×5 ml). The combined organic layers are dried (MgSO₄ or Alltech spice filter, Alltech Associates Inc. 2051 Waukegan Road, Deerfield, Ill. 60015) and the volatiles are removed under a stream of N₂.

Step 3

The residue is dissolved in CH₂Cl₂ (2 ml) and cooled to −78° C., upon which BBr₃ is added a 1.0M solution in CH₂Cl₂ (ca. 4.0 eq.) is added. The reaction is stirred overnight slowly warming to room temperature, upon which it is quenched with MeOH (5 ml). Saturated NaHCO₃ was added adjusting the pH of the solution to pH=8. The mixture is extracted with CH₂Cl₂ (1×10 ml). The organic layer is dried (MgSO₄ or Alltech spice filter) sand concentrated under a stream of N₂. Purification is achieved via recrystallization (hot MeOH or hot MeOH/EtOAc), preparative TLC or reverse phase HPLC to give the desired product.

Examples 30-45 were prepared according to the above procedure using the appropriate carboxylic acid as defined in each example.

### Example 30

4-12-[4-(2-Pyrrolidin-1-yl-ethoxy)-phenyl]-benzoimidazol-1-yl)-phenol

4-(2-pyrrolidin-1-yl-ethoxy)-benzoic acid can be prepared according to the reference Jones, Charles D.; Jevnikar, Mary G.; Pike, Andrew J.; Peters, Mary K.; Black, Larry J.; et al. J.Med.Chem.1984, 27 (8); 1057-1066.

4-{2-[4-(2-Pyrrolidin-1-yl-ethoxy)-phenyl]-benzoimidazol-1-yl}-phenol was prepared in according to the general procedure using 4-(2-pyrrolidin-1-yl-ethoxy)-benzoic acid as the carboxylic acid and the final product was purified by recrystallisation from hot MeOH. MS (M+1) 400.

### Example 31

4-[2-(2-Chloro-phenyl)-benzoimidazol-1-yl]-phenol

4-[2-(2-Chloro-phenyl)-benzoimidazol-1-yl]-phenol was prepared according to the general procedure using o-chlorobenzoic acid except that no purification of the final product was needed and polymer supported isocyanate beads were added during step 1.

MS (M+1) 321; ¹H NMR (CD₃OD) δ_{H} 7.88 (d, 1H),-7.44-7.68 (m, 7H), 7.24-7.27 (m, 2H), 6.83-6.86 (m, 2H).

### Example 32

4-(2-Isopropyl-benzoimidazol-1-yl)-phenol

4-(2-Isopropyl-benzoimidazol-1-yl)-phenol was prepared according to the general procedure using isobutyric acid except no purification of the final product was needed. MS (M+1) 253; ¹H NMR (CD₃OD) δ_{H} 7.80 (d, 1H), 7.56-7.60 (t, 1H), 7.49-7.53 (t,1H), 7.40-7.44 (m, 2H), 7.28 (d, 1H), 7.04-7.08 (m, 2H), 1.42 (d, 6H).

### Example 33

(+)-4-(2-sec-Butyl-benzoimidazol-1-yl)-phenol

4-(2-sec-Butyl-benzoimidazol-1-yl)-phenol was prepared according to the general procedure using (±)-2-methylbutyric acid, except no purification of the final product was needed. MS (M+1) 267; ¹H NMR (CD₃OD) δ_{H} 7.78 (d, 1H), 7.46-7.51 (t, 1H), 7.39-7.44 (t, 1H), 7.31-7.38 (m, 2H), 7.18-7.22 (d, 1H), 7.02-7.07 (m, 2H), 2.97-3.04 (m, 1H), 2.83-2.92 (m, 1H), 1.67-1.77 (m, 1H), 1.39 (d, 3H), 0.90-0.94 (t, 3H).

### Example 34

4-[2-(4-Iodo-phenyl)-benzoimidazol-1-yl]-phenol

4-[2-(4-Iodo-phenyl)-benzoimidazol-1-yl]-phenol was prepared according to the general procedure using 4-iodobenzoic acid, except that the final product was purified by recrystallisation from hot MeOH/EtOAc . MS (M+1)413; ¹H NMR (CDCl₃/CD₃OD) δ_{H} 7.61-7.70 (m, 2H), 7.55-7.59 (m, 2H), 7.09-7.26 (m, 4H), 6.97 (m, 2H), 6.80-6.82 (m, 2H).

### Example 35

4-(2-Cyclopropyl-benzoimidazol-1-yl)-phenol

4-(2-Cyclopropyl-benzoimidazol-1-yl)-phenol was prepared according to the general procedure using cyclopropanecarboxylic acid except the final product was purified by recrystallisation from hot MeOH. MS (M+1) 251; ¹H NMR (CD₃COCD₃) δ_{H} 8.97 (bs, 1H), 7.53-7.56 (m, 1H), 7.38-7.42 (m, 2H), 7.07-7.21 (m, 5H), 1.83-1.91 (m, 1H), 1.17-1.22 (m, 2H), 0.99-1.04 (m, 2H).

### Example 36

4-(2-Cyclopentyl-benzoimidazol-1-yl)-phenol 4-(2-Cyclopentyl-benzoimidazol-1-yl)-phenol was prepared according to the general procedure using.,cyclopentanecarboxylic acid except no purification of the final product was required and all solutions were dried using Alltech spice filters. MS (M+1) 279; ¹H NMR (CD₃OD) δ_{H} 7.82 (d, 1H), 7.51-7.63 (m, 2H), 7.42-7.46 (d, 2H), 7.30-7.33 (m, 1H), 7.08-7.12 (d, 2H), 3.32-3.43 (m, 1H), 2.12-2.21 (m, 2H), 1.88-2.03 (m, 4H), 1.72-1.81 (m, 2H).

### Example 37

4-[2-(5-Bromo-thiophen-2-yl)-benzoimidazol-1-yl]-phenol

4-[2-(5-Bromo-thiophen-2-yl)-benzoimidazol-1-yl]-phenol was prepared according to the general procedure using 5-bromo-thiophene-2-carboxylic acid except that the final product was purified by recrystallisation from hot MeOH and Alltech spice filters were used to dry solutions.

### Example 38

4-(2-Ethyl-benzoimidazol-1-yl)-phenol

4-(2-Ethyl-benzoimidazol-1-yl)-phenol

4-(2-Ethyl-benzoimidazol-1-yl)-phenol was prepared according to the general procedure except that propionyl chloride was used instead of a carboxylic acid, all solutions were died using Alltech spice filters and the final product was purified using reverse phase HPLC. MS (MH)⁺ 239; ¹H NMR (acetone) δ_{H} 7.63-7.66 (d, 1H), 7.32-7.35 (d, 2H), 7.15-7.24 (m, 2H), 7.06-7.12 (m, 3H), 2.73-2.81 (m, 2H), 1.28-1.33 (m, 3H).

### Example 39

4-(2-Trifluoromethyl-benzoimidazol-1-yl)-phenol

4-(2-Trifluoromethyl-benzoimidazol-1-yl)-phenol was prepared according to the general procedure using trifluoroacetic acid except that the final product was purified by reverse phase HPLC and MgSO₄ was used to dry all solutions. MS (MH)⁺ 279; ¹H NMR (acetone) δ_{H} 9.03 (s, 1H), 7.86-7.89 (m, 1H), 7.42-7.46 (m, 4H), 7.20-7.23 (m, 1H), 7.10-7.13 (d, 2H).

### Example 40

4-(2-Cyclopent-1-enyl-benzoimidazol-1-yl)-phenol

4-(2-Cyclopent-i-enyl-benzoimidazol-1-yl)-phenol was prepared according to the general procedure using cyclopent-1-enecarboxylic acid except that MgSO₄ was used to dry all solutions and the final product was purified by reverse phase HPLC. MS (MH)⁺ 277; ¹H NMR (CD₃OD) δ_{H} 7.67-7.70 (d, 1H), 7.20-7.32 (m, 4H), 7.00-7.07 (m, 3H), 5.82-5.85 (m, 1H), 2.76-2.83 (m, 2H), 2.42-2.50 (m, 2H), 1.90-2.00 (m, 2H).

### Example 41

4-(2-Cyclobutyl-benzoimidazol-1-yl)-phenol

4-(2-Cyclobutyl-benzoimidazol-1-yl)-phenol was prepared according to the general procedure using cyclobutanecarboxylic acid except that the final product crystallized from CH₂Cl₂ and required no further purification. MS (MH)⁺ 265; ¹H NMR (CD₃OD) δ_{H} 7.67-7.69 (d, 1H), 7.19-7.30 (m, 4H), 7.07-7.10 (d, 1H), 6.99-7.02 (d, 2H), 3.61-3.73 (m, 1H), 2.46-2.59 (m, 2H), 2.18-2.27 (m, 2H), 1.94-2.13 (m, 2H).

### Example 42

4-[2-(4-trifluoromethoxy-phenyl)-benzoimidazol-1-yl]-phenol

Step B

1-(4-Methoxy-phenyl)-2-(4-trifluoromethoxy-phenyl)-1H-benzoimidazole

4-[2-(4-trifluoromethoxy-phenyl)-benzoimidazol-1-yl]-phenol was prepared according to the general procedure using 4-(trifluoromethoxy) benzoic acid except that no purification of the final product was required. MS (MH)⁺ 371; ¹H NMR (CD₃OD) δ_{H} 7.74-7.76 (d, 1H), 7.64-7.66 (m, 2H), 7.32-7.38 (m, 2H), 7.28-7.30 (d, 2H), 7.23-7.25 (d, 1H), 7.17-7.19 (m, 2H), 6.91-6.93 (m, 2H).

### Example 43

1-(4-[l-(4-Hydroxy-phenyl)-1H-benzoimidazol-2-yl]-phenyl}-ethanone

1-{4-[1-(4-Hydroxy-phenyl)-1H-benzoimidazol-2-yl]-phenyl}-ethanone was prepared according to the general procedure using 4-acetyl benzoic acid, except that the final product was purified using flash chromatography (SiO₂). MS (MH)⁺ 329; ¹H NMR (CD₃OD) δ_{H} 7.94-7.97 (d, 2H), 7.74-7.76 (d, 1H), 7.67-7.69 (d, 2H), 7.28-7.36 (m, 2H), 7.22-7.24 (d, 1H), 7.15-7.17 (m, 2H), 6.90-6.92 (m, 2H), 2.57 (s, 3H).

### Example 44

4-[1-(4-Hyroxy-phenyl)-1H-benzoimidazol-2-yl]-benzonitrile

4-[1-(4-Hyroxy-phenyl)-1H-benzoimidazol-2-yl]-benzonitrile was prepared according to the general procedure using 4-cyano benzoic acid, except that the product of step 2 was purified by preparative TLC, all solutions were dried using Na₂SO₄ and the final product crystallized upon standing requiring-no further purification. MS (MH)⁺ 312; ¹H NMR (d₆-dmso) δ_{H} 9.98 (s, 1H), 7.83-7.85 (m, 2H), 7.77-7.79 (d, 1H), 7.68-7.70 (m, 2H), 7.25-7.32 (m, 2H), 7.22-7.24 (m, 2H), 7.14-7.16 (d, 1H), 6.88-6.91 (d, 2H).

### Example 45

4-[2-(4-Vinyl-phenyl)-benzoimidazol-1-yl]-phenol

4-[2-(4-Vinyl-phenyl)-benzoimidazol-1-yl]-phenol was prepared according to the general procedure using 4-vinyl benzoic acid except that the product of step 2 was purified by preparative TLC, all solutions were dried using Na₂SO₄ and the final product did not require purification. MS (MH)⁺ 313; ¹H NMR (d₆-dmso) δ_{H} 9.95 (s, 1H), 7.73-7.75 (d, 1H), 7.47-7.56 (m, 4H), 7.22-7.29 (m, 4H), 7.09-7.11 (d, 1H), 6.89-6.91 (d, 2H), 5.49-5.53 (m, 1H), 4.33-4.38 (t, 1H), 4.19-4.23 (m, 1H).

### Example 46

4-[2-(2-Chloro-phenyl)-benzoimidazol-1-yl]-3-methyl-phenol

2-Chloro-N-[2-(4-methoxy-2-methyl-phenylamino)-phenyl]-benzamide

To a solution of N-(4-methoxy-2-methyl-phenyl)-benzene-1,2-diamine (0.200 g, 0.877 mmol), 2-chlorobenzoic acid (0.205 g, 1.32 mmol), triethylamine (0.613 mL, 4.39 mmol), and a catalytic amount of 4-dimethylaminopyridine in of methylene chloride (2 mL), was added 1-propanephosphonic acid cyclic anhydride (1.05 mL, 1.75 mmol) as a 50% solution in ethyl acetate. The reaction mixture was stirred overnight at room temperature. To remove any unreacted amine, of PS-Isocyanate (150 mg) scavenger beads were added and the mixture was stirred for 1 hour before filtering off the beads. Additional isocyanate beads (300 mg) were added and stirring continued overnight. The beads were again filtered. The reaction material was diluted with methylene chloride, washed with saturated sodium bicarbonate and extracted into methylene chloride. The combined organic material was dried over magnesium sulfate, filtered, and concentrated, giving 2-chloro-N-[2-(4-methoxy-2-methyl-phenylamino)-phenyl]-benzamide. MS (MH)⁺ 367.

2-(2-Chloro-phenyl)-1-(4-methoxy-2-methyl-phenyl)-1H-benzoimidazole

To a solution of 2-chloro-N-[2-(4-methoxy-2-methyl-phenylamino)-phenyl]-benzamide in methanol (7 mL) was added 4N HCl as a solution in 1,4-dioxane (1.1 mL, 4.39 moles) and the solution was stirred at 60° C. overnight. Additional HCl (1 mL) and MeOH (1 mL) were added and the solution was stirred at 60° C. overnight again. The reaction material was neutralized with saturated sodium bicarbonate and extracted into methylene chloride. The organic extract was dried over magnesium sulfate, filtered and concentrated. Purification by Biotage flash chromatography (SiO₂), eluting with methylene chloride, gave 2-(2-chloro-phenyl) 1-(4-methoxy-2-methyl-phenyl)-1H-benzoimidazole (0.044 g, 14% yield over two steps). MS (MH)⁺ 349; ¹H NMR (CDCl₃) δ_{H} 7.86-7.88 (d, 1H), 7.25-7.33 (t, 1H), 7.18-7.24 (m, 3H), 6.99-7.11 (m, 4H), 6.70-6.74 (m, 2H), 3.76 (s, 3H), 2.38 (s, 3H), 1.89 (s, 3H).

4-[2-(2-Chloro-phenyl)-benzoimidazol-1-yl]-3-methyl-phenol

To a solution of 2-(2-chloro-phenyl)-1-(4-methoxy-2-methyl-phenyl)-1H-benzoimidazole (0.044 g, 0.131 mmol) in methylene chloride (2 ml) at −78° C. under nitrogen was added 1 M boron tribromide (1.34 mL, 1.34 mmol) as a solution in CH₂Cl₂ and the reaction was stirred as the solution warmed to room temperature overnight. The temperature was again brought to −78° C. and the reaction was quenched with methanol(0.3 mL). Upon warming to room temperature, the reaction was neutralized with saturated sodium bicarbonate and extracted with methylene chloride. The combined organic material was dried over magnesium sulfate, filtered, and concentrated, yielding 4-[2-(2-Chloro-phenyl)-benzoimidazol-1-yl]-3-methyl-phenol (0.091 g, 0.272 mmol). MS (MH)⁺ 335; ¹H NMR (CD₃OD) δ_{H} 7.97-7.99 (d, 1H), 7.62-7.78 (m, 5H), 7.41-7.49 (m, 2H), 7.30-7.32 (d, 1H), 6.78-6.79 (d, 1H), 6.71-6.74 (d, 1H), 1.96 (s, 1H).

### Example 47

3-Methyl-4-(2-o-tolyl-benzoimidazol-1-yl)-phenol

N-[2-(4-Methoxy-2-methyl-phenylamino)-phenyl]-2-methyl-benzamide

To a solution of N-(4-methoxy-2-methyl-phenyl)-benzene-1,2-diamine (0.200 g, 0.877 mmol), o-toluic acid (0.178 g, 1.3155 mmol), triethylamine (0.613 mL, 4.385 mmol), and a catalytic amount of 4-dimethylaminopyridine in methylene chloride (2 mL), was added 1-propanephosphonic acid cyclic anhydride (1.05 mL, 1.754 mmol) as a 50% solution in ethyl acetate. The reaction mixture was stirred overnight at room temperature. To remove any unreacted amine, PS-Isocyanate (0.150 g) scavenger beads were added and the mixture was stirred for 1 hour before filtering off the beads. Additional isocyanate beads (0.300 g) were added and stirring continued overnight. The beads were again filtered. The reaction material was diluted with methylene chloride, washed with saturated sodium bicarbonate, and extracted into methylene chloride. The combined organic material was dried over magnesium sulfate, filtered, and concentrated, giving N-[2-(4-methoxy-2-methyl-phenylamino)-phenyl]-2-methyl-benzamide. MS (MH)⁺ 347.

1-(4-Methoxy-2-methyl-phenyl)-2-o-tolyl-1H-benzoimidazole

To the crude N-[2-(4-methoxy-2-methyl-phenylamino)-phenyl]-2-methyl-benzamide dissolved in of anhydrous methanol (7 mL) was added 4 M HCl in 1,4-dioxane (1.1 mL, 4.385 moles) and the solution was stirred at 60° C. overnight. Additional HCl (1 mL) and MeOH (1 mL) were added and the solution was stirred at 60° C. overnight again. The reaction material was neutralized with saturated sodium bicarbonate and extracted into methylene chloride. The organic extract was dried over magnesium sulfate, filtered and concentrated. Purification by Biotage flash chromatography (SiO₂), eluting with 50% hexane in methylene chloride followed by methylene chloride then 50% ethyl acetate in methylene chloride, gave of 1-(4-methoxy-2-methyl-phenyl)-2-o-tolyl-1H-benzoimidazole (0.150 g, 49%/o yield over two steps). MS (MH)⁺ 329; ¹H NMR (CDCl₃) δ_{H} 7.86-7.88 (d, 1H), 7.25-7.33 (t, 1H), 7.18-7.24 (m, 3H), 6.99-7.11 (m, 4H), 6.70-6.74 (m, 2H), 3.76 (s, 3H), 2.38 (s, 3H), 1.89 (s, 3H).

3-Methyl-4-(2-o-tolyl-benzoimidazol-1-yl)-phenol

To of 1-(4-methoxy-2-methyl-phenyl)-2-o-tolyl-1H-benzoimidazole (0.155 g 0.473 mmol) in methylene chloride (2 ml) at −78° C. under nitrogen was added 1 M boron tribromide (1.34 mL, 1.34 mmol) and the reaction was stirred as the solution warmed to room temperature overnight. The temperature was again brought to −78° C. and the reaction was quenched with 0.3 mL of methanol. At room temperature, the reaction was neutralized with saturated sodium, bicarbonate and extracted with methylene chloride. The combined organic material was dried over magnesium sulfate, filtered, and concentrated, yielding 3-methyl4-(2-o-tolyl-benzoimidazol-1-yl)-phenol (0.143 g). MS (MH)⁺ 315; ¹H NMR (CD₃OD) δ_{H} 7.95-7.98 (d, 1H), 7.71-7.75 (t, 1H), 7.64-7.68 (t, 1H), 7.49-7.53 (t, 1H), 7.42-7.45 (d, 2H), 7.37-7.40 (d, 1H), 7.25-7.30 (m, 2H), 6.78-6.79 (d, 1H), 6.71-6.74 (d, 1H), 2.39 (s, 3H), 1.94 (s, 3H).

### Example 48

4-[2-(1-Methyl-cyclopropyl)-benzoimidazol-1-yl]-phenol

Step A

1-Methyl-cyclopropanecarboxylic acid [2-(4-methoxy-phenylamino)-phenyl]-amide

To a solution of N-(4-methoxy-phenyl)-benzene-1,2-diamine di hydrochloride salt (0.286 g, 1.00 mmol), 1-methyl-cyclopropanecarboxylic acid (0.200 g, 2.0 mmol), Et₃N (0.7 ml, 5.0 mmol) and DMAP (cat.) in CH₂Cl₂ (5 ml) was added PPM (50% solution in EtOAc, 1.32 ml, 2.2 mmol). The reaction was stirred at RT for 48 hours. The reaction mixture was concentrated under a stream of N₂ and was used directly in the next step. MS 297 (M+1).

1-(4-Methoxy-phenyl)-2-(1-methyl-cyclopropyl)-1H-benzoimidazole

To a solution of crude 1-methyl-cyclopropanecarboxylic acid [2-(4-methoxy-phenylamino)-phenyl]-amide in THF (10 ml) was added HCl as a 4.0M solution in 1,4 dioxane (2.5 ml, 2.5 mmol) and was heated at 60° C. overnight, upon which a precipitate formed. MeOH (10 ml) was added to the mixture, the precipitate dissolved and heating at 60 C. was continued for 24 hours. The reaction was allowed to cool to RT, diluted with CH₂Cl₂ (20 ml) and washed with 10% K₂CO₃ until pH of aqueous solutions remained ca.10-12. The organic layer was dried (MgSO₄), filtered and concentrated under a stream of N₂. The residue was subjected to flash chromatography (SiO₂, biotage, EtOAc:hexanes 1:5) to give the desired product as an oil (0.270 g, 0.967 mmol, 97% over two steps). ¹H NMR 400 MHz (CDCl₃) δ_{H} 7.73 (1H, m), 7.32 (2H, m), 7.24-7.13 (2H, m), 7.07-7.00 (3H, m), 3.88 (3H, s), 1.23 (2H, m), 1.14 (3H, s) and 0.68 (2H, m). MS 279 (M+1).

4-[2-(1-Methyl-cyclopropyl)-benzoimidazol-1-yl]-phenol

To a solution of 1-(4-methoxy-phenyl)-2-(1-methyl-cyclopropyl)-1H-benzoimidazole (0.280 g, 1.00 mmol) in CH₂Cl₂ (5 ml) cooled to −78 ° C. was added BBr₃ as a 1.0M solution in CH₂Cl₂ (3.0 ml, 3.0 mmol ). The reaction was stirred overnight, slowly warming to RT, re-cooled to −78° C., quenched with MeOH (5 ml), diluted with CH₂Cl₂ (20 ml) and washed with sat. NaHCO₃ (1×10 ml). The aqueous washing was back extracted with CH₂Cl₂ (1×20 ml). The combined organics were dried (MgSO₄), filtered and concentrated by vacuum. The residue was subjected to flash chromatography (SiO₂, biotage, EtOAc:hexanes 2:3) to give the desired product. ¹H NMR 400 MHz (CD₃OD) δ_{H} 7.58 (1H, m), 7.30−7.18 (4H, m), 7.04−6.97 (3H, m), 1.20 (3H, s), 1.17 (2H, m) and 0.68 (2H, m). MS 265 (M+1).

### Example 49

4-(2-Cyclopropvlmethyl-benzoimidazol-1-yl)-phenol

Step A

2-Cyclopropyl-N-[2-(4-methoxy-phenylamino)-phenyl]-acetamide

To a solution of N-(4-methoxy-phenyl)-benzene-1,2-diamine di hydrochloride salt (0.286 g, 1.00 mmol), cyclopropyl-acetic acid (0.200 g, 2.0 mmol), Et3N (0.7 ml, 5.0 mmol) and DMAP (cat.) in CH2Cl2 (5 ml) was added PPM (50% solution in EtOAc, 1.32 ml, 2.2 mmol). The reaction was stirred at RT for 48 hours, then polymer supported isocyanate beads (Argonaut technologies 1.7 mmol/g loading, 0.200 g) was added. The reaction mixture was stirred for a overnight, filtered, concentrated under a stream of N₂ and was used directly in the next step.

Step B

2-Cyclopropylmethyl-1-(4-methoxy-phenyl)-1H-benzoimidazole

To a solution of crude 2-cyclopropyl-N-[2-(4-methoxy-phenylamino)-phenyl]-acetamide in THF (10 ml) was added HCl as a 4.0M solution in 1,4 dioxane (2.5 ml, 2.5 mmol) and was heated at 60° C. overnight, upon which a precipitate formed. MeOH (10 ml) was added to the mixture, the precipitate dissolved and heating at 60° C. was continued for 24 hours. The reaction was allowed to cool to RT, diluted with CH₂Cl₂ (20 ml) and washed with 10% K₂CO₃ until pH of aqueous solutions remained ca.10-12. The organic layer was dried (MgSO₄), filtered and concentrated under a stream of N₂. The residue was subjected to flash chromatography (SiO₂, biotage, EtOAc:hexanes 1:5) to give the desired product as an oil (0.215 g, 0.77 mmol, 77% over two steps). ¹H NMR 400 MHz (CDCl₃) δ_{H} 7.79 (1H, d J 8.0 Hz), 7.28−7.23 (3H, m), 7.18−7.07−7.01 (4H, m), 3.89 (3H, s), 2.70 (2H, d J 7.0 Hz), 1.09 (1H, m), 0.48 (2H, m) and 0.10 (2H, m). MS 279 (M+1).

Step C.

4-(2-Cyclopropylmethyl-benzoimidazol-1-yl)-phenol

To a solution of 2-cyclopropylmethyl-1-(4-methoxy-phenyl)-1H-benzoimidazole (0.210 g, 1.00 mmol) in CH₂Cl₂ (5 ml) cooled to -78 ° C. was added BBr₃ as a 1.0M solution in CH₂Cl₂ (2.3 ml, 2.3 mmol ). The reaction was stirred overnight, slowly warming to RT, re-cooled to −78 ° C., quenched with MeOH (5 ml), diluted with CH₂Cl₂ (20 ml) and washed with sat. NaHCO₃ (1×10 ml). The aqueous washing was back extracted with CH₂Cl₂ (1×20 ml). The combined organics were dried (MgSO₄), filtered and concentrated by vacuum. The residue was subjected to flash chromatography (SiO₂, biotage, EtOAc:hexanes 2:3) to give the desired product. ¹H NMR 400 MHz (CD₃OD) δ_{H} 7.60 (1H, m), 7.23−7.18 (4H, m), 7.05 (1H, m), 6.97 (2H, d J9.0 Hz), 2.67 (2H, d J 7.0 Hz), 0.98 (1H, m), 0.45 (2H, m) and 0.08 (2H, m).

### Example 50

4-(1-Thiophen-3-yl-1H-benzoimidazol-2-yl)-phenol

N-(2-Amino-phenyl)4-methoxy-benzamide

To a suspension of o-phenylenediamine dihydrochloride (10 g, 33.3 mmol) in methylene chloride (200 mL) was added triethylamine (16.25 mL,1 16.7 mmol) and a catalytic amount of 4-dimethylaminopyridine. The suspension was cooled to 0° C. and 5.68 (33.3 mmol) of p-anisoyl chloride was added. The reaction material was stirred at room temperature and under nitrogen over night. The solution was evaporated but the material was not purified. MS indicated the desired monoacyl compound [(MH)⁺ 243] in addition to a diacyl side product and the ring closed benzimidazole.

2-(4-Methoxy-phenyl)-1H-benzoimidazole

To the crude N-(2-amino-phenyl)-4-methoxy-benzamide was added of acetic acid (100 mL) and the reaction material was heated at 85-90° C. for 48 h. The reaction was incomplete and the, acetic acid was evaporation. To the crude residue was added H₂O (50 mL)and the solution was neutralized to pH 6 with a 1 N solution of sodium hydroxide. The organic material was extracted with ethyl acetate and the combined organic extracts were dried over magnesium sulfate and concentrated to an oil. Over 3 days, a white crystalline solid formed in the oil and the mixture was decanted. The white solid, the diacyl compound, was filtered and washed with ethyl acetate and acetone. The filtrate and decanted oil were combined. Additional white solid precipitated and was filtered. The concentrated filtrate was dissolved in a mixture of methylene chloride and ethyl acetate (30 mL, 1:1 v/v) and methanol (2 mL). The solution was cooled over night in a refrigerator and feathery white crystals (desired product) formed and were filtered. The filtrate was concentrated and the residue was dissolved in methylene chloride (10 mL) and ethyl acetate (5 mL). Methylene chloride was slowly removed on the rotary evaporator until cloudy. The material was swirled to redissolve contents and crystallization was induced by scratching the glass surface. The crystals were filtered and washed with ethyl acetate. Combining all crops gave 2-(4-methoxy-phenyl)-1H-benzoimidazole (4 g). MS (MH)⁺ 225; ¹H NMR (acetone) δ_{H} 8.14-8.16 (d, 2H), 7.52-7.54 (m, 2H), 7.15-7.18 (m, 2H), 7.07-7.09 (d, 2H), 3.88 (s, 3H).

4-(1-Thiophen-3-yl-1H-benzoimidazol-2-yl)-phenol

Step A

2-(4-Methoxy-phenyl)-1-thiophen-3-yl-1H-benzoimidazole

To a flame dried flask under nitrogen was added 2-(4-Methoxy-phenyl)-1H-benzoimidazole(0.400 g (1.78 mmol), of 3-Iodothiophene (0.18 mL, 1.63 mmol), 40 mg (0.08 mmol) of copper (I) trifluoromethane sulfonate benzene, 580 mg (1.78 mmol) of cesium carbonate, 292 mg (1.62 mmol) of 1,10-phenanthroline, 187 mg (0.80 mmol) of trans, trans-dibenzylidine acetone, and 3 mL of xylenes. The reaction mixture was heated to 125° C. for 48 h. TLC indicated mostly starting material and 450 mg of cesium carbonate from a new source was added. The material was stirred while heating at 165° C. for 4 h. No change was seen by TLC and heating continued over weekend. The solvent was stripped and purification of the crude residue by silica gel flash chromatography, eluting with diethyl ether, gave 14 mg of an impure yellow oil. Further purification be preparatory TLC (1.0 mm), eluting with diethyl ether, gave 7 mg of pure 2-(4-Methoxy-phenyl)-1-thiophen-3-yl-1H-benzoimidazole. MS (MH)⁺ 307; ¹H NMR (acetone) δ_{H} 7.64-7.72 (m, 3H), 7.56-7.58 (d, 2H), 7.20-7.26 (m, 3H), 7.10-7.12 (d, 1H), 6.90-6.92 (d, 2H), 3.81 (s, 3H). To 7.0 mg of the above product was added 2.5 mL of methylene chloride.

Step B

4-(1-Thiophen-3-yl-1H-benzoimidazol-2-yl)-phenol

To the solution of 2-(4-methoxy-phenyl)-1-thiophen-3-yl-1H-benzoimidazole in methylene chloride cooled to 0° C. was added boron tribromide (0.08 mL) as a 1.0M solution in methylene chloride dropwise by syringe. The reaction material was stirred at room temperature over night. After quenching with methanol, the solvent was stripped and the crude residue was dissolved in ethyl acetate and washed with sodium bicarbonate and sodium chloride. Purification by preparatory TLC (1.0 mm), and eluting with diethyl ether, gave 7.4 mg of pure title compound. MS (MH)⁺ 293; ¹H NMR (CD₃OD) δ_{H} 7.66-7.68 (d, 1H), 7.60-7.62 (d, 1H), 7.53-7.54 (d, 1H), 7.38-7.40 (d, 2H), 7.25-7.30 (m, 3H), 7.01-7.02 (d,1H).

### Example 51

4-(1-Thiophen-2-yl-1H-benzoimidazol-2-yl)-phenol

Step A

2-(4-Methoxy-phenyl)-1-thiophen-2-yl-1H-benzoimidazole

2-(4-Methoxy-phenyl)-1-thiophen-2-yl-1H-benzoimidazole was prepared in a manner analogous to that as described in example 50 step A except that 2-iodo thiophene was used instead of 3-Iodothiophene. MS (MH)⁺ 307; ¹H NMR (CD₃OD) δ_{H} 7.71-7.73 (d, 1H), 7.54-7.60 (m, 3H), 7.27-7.37 (m, 3H), 7.14-7.18 (m, 2H), 6.92-6.95 (d, 2H), 3.81 (s, 3H).

4-(1-Thiophen-2-yl-1H-benzoimidazol-2-yl)-phenol

Deprotection was effected following the method in Example 50 step B except that 2-(4-Methoxy-phenyl)-1-thiophen-2-yl-1H-benzoimidazole was used instead of 2-(4-methoxy-phenyl)-1-thiophen-3-yl-1H-benzoimidazole. Trituration with minimal acetone, followed by washes with diethyl ether and hexane, gave 4-(1-Thiophen-2-yl-1H-benzoimidazol-2-yl)-phenol (0.027 g, 92.5 μmol). MS (MH)⁺ 293; ¹H NMR (d₆-dmso) δ_{H} 9.92 (s, 1H), 7.65-7.69 (m, 2H), 7.42-7.45 (d, 2H), 7.21-7.28 (m, 3H), 7.14-7.17 (m, 2H), 6.71-6.73 (d, 2H).

### Example 52

4-[1-(4-Hydroxy-phenyl)-1H-benzoimidazol-2-yl]-benzoic acid methyl ester

N-[2-(4-Methoxy-phenylamino)-phenyl]-terephthalamic acid methyl ester

To a solution of N-(4-methoxy-phenyl)-benzene-1,2-diamine bis hydrochloride (23.0 g 0.08 mol), monomethylterephthalate (17.3 g, 0.096 mol), triethylamine (112 ml, 0.80 mol) and 4-dimethylaminopyridine (1.00 g, 8.29 mal) in methylene chloride (500 ml), was added) of 1-propanephosphonic acid cyclic anhydride (50% in ethyl acetate, 72 ml, 0.120 mol). The reaction mixture was stirred under nitrogen over night, and was then diluted with ethyl acetate (1.1 L) and washed twice with saturated sodium bicarbonate (2×250 ml). The pooled aqueous washes were extracted with ethyl acetate (300 ml), and the combined organic extracts were washed with brine (300 ml), dried (MgSO₄) and concentrated by vacuum. MS (MH)⁺ 377; ¹H NMR (CDCl₃) δ_{H} 8.41 (s, 1H), 8.00-8.09 (m, 3H), 7.71-7.82 (d, 1H), 7.68-7.70 (d, 2), 7.34-7.36 (m, 1H), 7.09-7.15 (m, 3H), 3.90 (s, 3H), 3.73 (s, 3H).

4-[1-(4-Methoxy-phenyl)-1H-benzoimidazol-2-yl]-benzoic acid methyl ester

N-[2-(4-methoxy-phenylamino)-phenyl]-terephthalamic acid methyl ester (28 g, 0.074 mol) was heated in glacial acetic acid (225 ml) at 80° C. under an atmosphere of nitrogen over night. Once the reaction material cooled to room temperature, heptane was added (400 ml) and some of the acetic acid/heptane mixture was evaporated. Additional heptane (2×200 ml) was added and the remaining acetic acid was evaporated. The resulting light brown solid was triturated with Et₂O and the product was filtered as a white solid (14.4 g, 40.2 mmol) of pure title compound. MS (MH)⁺ 359; ¹H NMR (CDCl₃) δ_{H} 7.94-7.96 (d, 2H), 7.85-7.87 (d, 1H), 7.63-7.66 (d, 2H), 7.18-7.34 (m, 5H), 6.97-6.99 (d, 2H), 3.88 (s, 3H), 3.86 (s, 3H).

4-[1-(4-Hydroxy-phenyl)-1H-benzoimidazol-2-yl]-benzoic acid methyl ester

To a solution of 4-[1-(4-methoxy-phenyl)-1H-benzoimidazol-2-yl]-benzoic acid methyl ester (1.1 g, 3.07 mmol) in CH₂Cl₂ (12 ml) cooled to −78° C. under an atmosphere of nitrogen, was added boron tribromide (7 ml, 7 mmol) as a 1 M solution in CH₂Cl₂. The reaction material was stirred over night while slowly warming to room temperature. Methanol (5 ml) was added to the reaction material and stirring was continued for 3 h. The reaction was neutralized to pH7 with saturated sodium bicarbonate and diluted with ethyl acetate (25 ml), forming a white solid in the process. After filtering the solid, the organic phase of the biphasic filtrate was washed with saturated sodium bicarbonate and the brine. The organic extract was then dried (MgSO₄) and concentrated by vacuum. The crude residue was triturated with diethyl ether to give the title compound (0.376 g, 1.09 mmol). MS (MH)⁺ 345; ¹H NMR (CDCl₃) δ_{H} 7.92-7.94 (d, 2H), 7.82-7.84 (d, 1H), 7.61-7.63 (d, 2H), 7.18-7.33 (m, 3H), 7.05-7.07 (d, 2H), 6.88-6.91 (d, 2H), 3.85 (s, 3H).

### Example 53

4-[1-(4-Hydroxy-phenyl)-1H-benzoimidazol-2-yl]-benzoic acid ethyl ester

To a solution of 4-[1-(4-methoxy-phenyl)-1H-benzoimidazol-2-yl]-benzoic acid methyl ester (0.38 g 0.11 mmol) in CH₂Cl₂ (2 ml) cooled to −78° C. was added boron tribromide (0.44 ml, 0.44 mol) as a 1.0M solution in methylene chloride. The mixture was allowed to stir over night, slowly warming to room temperature. Ethanol (1 ml) was added and stirring continued for 3 h. The reaction mixture was brought to pH7 with saturated sodium bicarbonate and extracted with methylene chloride (15 ml). The organic phase was washed once with saturated sodium bicarbonate (3 ml), once with brine (5 ml), and then dried (MgSO₄) and concentrated by vacuum. The crude residue was purified by preparatory thin layer chromatography (0.5 mm), eluting with 25% ethyl acetate in hexane, to give the title compound (0.010 g, 27.9 μmol). MS (MH)⁺ 358.8; ¹H NMR (CDCl₃) δ_{H} 7.90-7.91 (d, 2H), 7.75-7.77 (d, 1H), 7.56-7.58 (d, 2H), 7.14-7.29 (m, 3H), 7.02-7.04 (d, 2H), 6.84-6.86 (d, 2H), 4.25-4.31 (m, 2H), 1.28-1.31 (m, 3H).

### Example 54

4-[l-(4-Hydroxy-phenyl)-1H-benzoimidazol-2-yl]-benzoic acid isopropyl ester

Following the method outlined in Example 53, except replacing ethanol with isopropyl alcohol yielded title compound. MS (MH)⁺ 373; ¹H NMR (CDCl₃) δ_{H} 7.94-7.96 (d, 2H), 7.85-7.887 (d, 1H), 7.63-7.65 (d, 2H), 7.20-7.34 (m, 3H), 7.13-7.15 (d, 2H), 6.92-6.94 (d, 2H), 5.18-5.23 (m,1H), 1.32-1.34 (d, 6H).

Synthesis of hydroxy-protected 4-[1-(4-Hydroxy-phenyl)-1H-benzoimidazol-2-yl]-benzoic acids

4-[1-(4-Methoxy-phenyl)-1H-benzoimidazol-2-yl]-benzoic acid

To a solution of 4-[1-(4-methoxy-phenyl)-1H-benzoimidazol-2-yl]-benzoic acid methyl ester (0.45 g, 1.26 mmol THF/MeOH (1:1, 12 ml) was added aqueous sodium hydroxide (1 g/5 ml water). The solution was stirred at room temperature over night. To this solution was added 1N hydrochloric (25 ml). After 15 minutes of stirring, the white solid was filtered (348 mg, 80 % yield) giving 4-[1-(4-methoxy-phenyl)-1H-benzoimidazol-2-yl]-benzoic acid. MS (MH)⁺ 345; ¹H NMR (CDCl₃) δ_{H} 7.89-7.92 (d, 2H), 7.75-7.77 (d, 1H), 7.52-7.55 (d, 2H), 7.19-7.29 (m, 2H), 7.11-7.16 (m, 3H), 6.91-6.93 (d, 2H), 3.79 (s, 3H).

4-[1-(4-Benzyloxy-phenyl)-1H-benzoimidazol-2-yl]-benzoic acid methyl ester

To a mixture of 4-[1-(4-hydroxy-phenyl)-1H-benzoimidazol-2-yl]-benzoic acid methyl ester (0.185 g, 0.537 mmol), potassium carbonate (0.148 g, 1.07 mmol) and a catalytic amount of potassium iodide in butanone (8 ml) was added benzyl bromide (83 μL, 0.698 mmol). The reaction mixture was stirred at reflux for 3 h and then cooled to room temperature. The reaction material was diluted with 20 ml of H₂O was extracted with ethyl acetate (3 ×30 ml. The combined organic extracts were washed with H₂O and then brine, and dried over magnesium sulfate and concentrated. The resulting orange solid was triturated with diethyl ether to give 4-[1-(4-benzyloxy-phenyl)-1H-benzoimidazol-2-yl]-benzoic acid methyl ester (0.180 g, 0.415 mmol). MS (MH)⁺ 435; ¹H NMR (CDCl₃) δ_{H} 7.97-7.99 (d, 2H), 7.90-7.92 (d, 1H), 7.67-7.69 (d, 2H), 7.28-7.48 (m, 8H), 7.21-7.23 (d, 2H), 7.07-7.09 (d, 2H), 5.12 (s, 2H), 3.91 (s, 3H).

4-[1-(4-Benzyloxy-phenyl)-1H-benzoimidazol-2-yl]-benzoic acid

To a solution of 4-[1-(4-Benzyloxy-phenyl)-1H-benzoimidazol-2-yl]-benzoic acid methyl ester (0.148 g, 0.341 mmol) methanol/THF (1:1, 2 ml) was added 5N sodium hydroxide (700 μL) and the reaction mixture was stirred over night. The solution was acidified to pH 2 with 1 N hydrochloric acid. The reaction mixture was diluted to 30 ml with ethyl acetate and washed with H₂O and the brine. The organic extract was dried (MgSO₄) and concentrated by vacuum to give 4-[1-(4-benzyloxy-phenyl)-1H-benzoimidazol-2-yl]-benzoic acid (0.088 g, 0.210 mmol). MS (MH)⁺ 421; ¹H NMR (CDCl₃) δ_{H} 7.94-7.96 (d, 2H), 7.81-7.83 (d, 1H), 7.58-7.60 (d, 2H), 7.15-7.43 (m, 10H), 7.02-7.05 (d, 2), 5.07 (s, 2H).

4-{1-[4-(Tetrahydro-pyran-2-yloxy)-phenyl]-1H-benzoimidazol-2-yl}-benzoic acid methyl ester

To a suspension of 4-[1-(4-hydroxy-phenyl)-1H-benzoimidazol-2-yl]-benzoic acid methyl ester (0.150 g, 0.436 mmol) of 2,3-dihydropyran (0.5 ml) was added two drops of concentrated sulfuric acid. To the reaction material was added THF (3 ml) and the mixture was-stirred at room temperature under nitrogen over night. The reaction material was diluted with ethyl acetate (30 ml) and washed with saturated sodium bicarbonate and brine. The organic phase was dried (MgSO₄) and concentrated by vacuum. The residue was purified by silica gel chromatography to give 4-{1-[4-(tetrahydro-pyran-2-yloxy)-phenyl-1H-benzoimidazol-2-yl}-benzoic acid methyl ester (0.151 g, 0.352 mmol). MS (MH)⁺ 429; ¹H NMR (CDCl₃) δ_{H} 7.94-7.96 (d, 2H), 7.85-7.87 (d, 1H), 7.64 (d, 2H), 7.12-7.34 (m, 7H), 5.44-5.46 (m, 1H), 3.89-6.93 (m, 1H), 3.88 (s, 3H), 3.63-3.66 (m, 1H), 1.61-2.02 (m, 6H). MeOH (1 ml) and stirring was continued at RT for 15 minutes. Sat. NaHCO₃ was added adjusting the pH to ca. 7. The mixture was extracted with EtOAc (2×5 ml). The combined EtOAc layers were washed with brine (1×3 ml), dried (Na2SO4), filtered and concentrated. The residue was purified by reverse phase HPLC (MeCN2:98 H₂O) to give N-benzhydryl4-[1-(4-hydroxy-phenyl)-1H-benzoimidazol-2-yl]-benzamide. MS (MH)⁺ 496; ¹H NMR (CD₃OD) δ_{H} 7.81-7.83 (d, 2H), 7.74-7.76 (d, 1H), 7.63-7.65 (d, 2H), 7.22-7.35 (m, 13H), 7.14-7.16 (d, 2H), 6.90-6.92 (d, 2H), 6.41 (s, 1H).

### Example 56

4-[1-(4-Hydroxy-phenyl)-1H-benzoimidazol-2-yl]-N-isopropyl-benzamide

4-[1-(4-benzyloxy-phenyl)-1H-benzoimidazol-2-yl]-N-isopropyl-benzamide

To a solution of 4-[1-(4-Benzyloxy-phenyl)-1H-benzoimidazol-2-yl]-benzoic acid (0.66 g, 0.16 mmol), of isopropylamine hydrochloride (0.013 g, 0.13 mmol), of triethylamine (0.15 ml, 1.04 mmol), and 4-dimethylaminopyridine (0.005 g) in CH₂Cl₂ (5 ml) was added 1-propanephosphonic acid cyclic anhydride (50% in EtOAc, 0.12 ml, 0.200 mmol). The solution was stirred at room temperature and under nitrogen for 17 h. The reaction material was diluted to with ethyl acetate (40 ml) and washed with saturated sodium bicarbonate and then brine. The organic phase was dried (MgSO₄) and concentrated by vacuum to give giving 4-[1-(4-benzyloxy-phenyl)-1H-benzoimidazol-2-yl]-N-isopropyl-benzamide (0.055 g, 0.1 19 mmol). MS (MH)⁺ 462.

4-[1-(4-Hydroxy-phenyl)-1H-benzoimidazol-2-yl]-N-isopropyl-benzamide

A mixture of 4-[1-(4-benzyloxy-phenyl)-1H-benzoimidazol-2-yl]-N-isopropyl-benzamide (0.055 g, 0.119 mmol), ammonium formate (0.075 g, 1.27 mmol) and a catalytic amount of palladium black in methanol (5 ml) was heated at 60° C. under an atmosphere of nitrogen for 17 h. The reaction material was filtered through a plug of diatomaceous earth, which was then washed with methanol. The filtrate was diluted with of ethyl acetate (40 ml), and washed with saturated sodium bicarbonate, H₂O, and brine. The organic phase was dried (MgSO₄) and concentrated by vacuum to give 4-[l-(4-hydroxy-phenyl)-1H-benzoimidazol-2-yl]-N-isopropyl-benzamide (0.040 g, 0.108 mmol). MS (MH)⁺ 372; ¹H NMR (CD₃OD) δ_{H} 7.72-7.74 (d, 1H), 7.67-7.70 (d, 2H), 7.53-7.55 (d, 2H), 7.18-7.30 (m, 3), 7.03-7.06 (d, 2H), 6.84-6.86 (d, 2H), 4.13l-4.16 (m, 1H), 1.17-1.19 (d, 6H).

### Example 57

N-Benzyl-4-[1-(4-hydroxy-phenyl)-1H-benzoimidazol-2-yl]-benzamide

N-Benzyl4-{l -[4-(tetrahydro-pyran-2-yloxy)-phenyl]-1H-benzoimidazol-2-yl}-benzamide

To a solution of 4-{1-[4-(tetrahydro-pyran-2-yloxy)-phenyl]-1H-benzoimidazol-2-yl}-benzoic acid (0.044 g, 0.107 mmol), benzyl amine (0.010 g, 0.09 mmol), Et₃N (0.100 ml, 0.72 mmol) and DMAP (catalytic amount) in CH₂Cl₂ (3 ml) was added PPAA (0.082 ml, 0.135 mmol) as a 50% solution in EtOAc. The reaction mixture was stirred O/N at RT. The mixture was diluted with EtOAc (40 ml) and washed with sat. NaHCO₃ (2×15 ml) and brine (1×15 ml). The organic layer was dried (MgSO₄), filtered and concentrated to give N-benzyl-4-{1-[4-(tetrahydro-pyran-2-yloxy)-phenyl]-1H-benzoimidazol-2-yl}-benzamide (0.040 g, 79.5 1mol). MS (MH)⁺ 504; ¹H NMR (CDCl₃) δ_{H} 7.79-7.81 (d, 1H), 7.65-7.67 (d, 2H), 7.09-7.32 (m, 11H), 6.79-6.81 (m, 1H), 5.42-5.43 (m, 1H), 4.58-4.59 (d, 2H), 3.87-3.93 (m, 1H), 3.60-3.63 (m, 1H), 1.94-2.00 (m, 1H), 1.85-1.87 (m, 2H), 1.58-1.72 (m, 3H);

N-Benzyl-4-[1-(4-hydroxy-phenyl)-1H-benzoimidazol-2-yl]-benzamide

To a solution of N-benzyl-4-{1-[4-(tetrahydro-pyran-2-yloxy)-phenyl]-1H-benzoimidazol-2-yl}-benzamide (0.040 g, 79.5 μmol) and of TFA (0.5 ml) in of methylene chloride (1 ml) was added triethylsilane (0.13 ml, 0.8 mmol) and the reaction mixture was stirred at room temperature and under nitrogen for 17 h. The solvent was evaporated and purification by preparatory thin layer chromatography (1 mm), eluting with 10% methanol in methylene chloride, gave N-benzyl4-[1-(4-hydroxy-phenyl)-1H-benzoimidazol-2-yl]-benzamide (0.007 g, 16.7 μmol). MS (MH)⁺ 420; ¹H NMR (CDCl₃) δ_{H} 7.90-7.92 (d, 1H), 7.64-7.70 (m, 4H), 7.56-7.58 (d, 2H), 7.44-7.49 (m, 3H), 7.37-7.41 (t, 1H), 7.22-7.28 (m, 3H), 7.04-7.06 (d, 2H), 6.88-6.90 (d, 2H), 4.53 (s, 2H).

### Example 58

4-[1-(4-Hydroxy-phenyl)-1H-benzoimidazol-2-yl]-N-(1-phenyl-ethyl)-benzamide

4-[1-(4-Methoxy-phenyl)-1H-benzoimidazol-2-yl]-N-(1-phenyl-ethyl)-benzamide

To a solution of 4-[1-(4-methoxy-phenyl)-1H-benzoimidazol-2-yl]-benzoic acid (0.073 g, 0.212 mmol), 1-phenyl-ethylamine (0.023 ml, 0.177 mmol), Et₃N (0.200 ml, 1.68 mmol) and DMAP (catalytic amount) in CH₂Cl₂ (5 ml) was added PPAA (0.160 ml, 0.265 mmol) as a 50% solution in EtOAc. The reaction was stirred at RT for O/N, diluted with EtOAc (30 ml) and washed with sat. NaHCO₃ (2×15 ml) and brine (1×15 ml). The organic layer was dried (MgSO₄), filtered and concentrated. The crude mixture was used directly in the next step.

4-[1-(4-Hydroxy-phenyl)-1H-benzoimidazol-2-yl]-N-(1-phenyl-ethyl)-benzamide

To a solution of crude 4-[1-(4-methoxy-phenyl)-1H-benzoimidazol-2-yl]-N-(1-phenyl-ethyl)-benzamide (0.186 mmol) in CH₂Cl₂ (1.5 ml) cooled to −78° C. under an atmosphere of N₂ was added BBr₃ (0.5 ml, 0.50 mmol) as a 1.0 M solution in CH₂Cl₂. The reaction was stirred overnight slowly warming to RT. The reaction was quenched by the addition of MeOH (0.5 ml) and stirring was continued at RT for 15 minutes. Sat. NaHCO₃ was added adjusting the pH to ca. 7. The mixture was extracted with EtOAc (3×20 ml). The combined EtOAc layers were washed with brine (1×3 ml), dried (MgSO₄), filtered and concentrated to give 4-[1-(4-hydroxy-phenyl)-1H-benzoimidazol-2-yl]-N-(1-phenyl-ethyl)-benzamide (0.049 g, 0.113 mmol). MS (MH)⁺ 434; ¹H NMR (CD₃OD) δ_{H} 7.80-7.82(d, 2H), 7.75-7.77 (d, 1H), 7.21-7.38 (m, 7H), 7.16-7.18 (d, 2H), 6.90-6.93 (d, 2H), 5.18-5.22 (m, 1H), 1.52-1.54 (d, 3H).

### Example 59

4-[1-(4-Hydroxy-phenyl)-1H-benzoimidazol-2-yl]-N-thiophen-2-ylmethyl-benzamide

4-[1-(4-Methoxy-phenyl)-1H-benzoimidazol-2-yl]-N-thiophen-2-ylmethyl-benzamide

To a solution of 4-[1-(4-methoxy-phenyl)-1H-benzoimidazol-2-yl]-benzoic acid (0.058 g, 0.168 mmol), C-thiophen-2-yl-methylamine (0.023 g, 0.202 mmol), triethylamine (0.235 ml, 1.68 mmol), and 4-dimethylaminopyridine (catalytic amount) in methylene chloride (2 ml) was added 50% 1-propanephosphonic acid cyclic anhydride in ethyl acetate (0.150 ml, 0.252 mmol). The solution was stirred for 48 h at room temperature and under nitrogen. Saturated sodium bicarbonate (3 ml) was added and the reaction mixture was extracted with ethyl acetate. The combined ethyl acetate extracts were washed with brine, dried over sodium sulfate and concentrated. The crude 4-[1-(3-methoxy-phenyl)-1H-benzoimidazol-2-yl]-N-thiophen-2-ylmethyl-benzamide was used in the next step without further purification. MS (MH)⁺ 440.

4-[1-(4-Hydroxy-phenyl)-1H-benzoimidazol-2-yl]-N-thiophen-2-ylmethyl-benzamide

To a solution of crude 4-[1-(3-methoxy-phenyl)-1H-benzoimidazol-2-yl]-N-thiophen-2-ylmethyl-benzamide (0.168 mmol) in CH₂Cl₂ (1 ml) cooled to −78° C. under an atmosphere of N₂ was added BBr3 (1.0 ml, 1.0 mmol) as a 1.0 M solution in CH₂Cl₂. The reaction was stirred overnight slowly warming to RT. The reaction was quenched by the addition of MeOH (1 ml) and stirring was continued at RT for 15 minutes. Sat. NaHCO₃ was added adjusting the pH to ca. 7. The mixture was extracted with EtOAc (2×5 ml). The combined EtOAc layers were washed with brine (1×3 ml), dried (Na₂SO₄), filtered and concentrated. The residue was purified by reverse phase HPLC (MeCN2:98 H₂O) to 4-[1-(4-Hydroxy-phenyl)-1H-benzoimidazol-2-yl]-N-thiopen-2-ylmethyl-benzamide. MS (MH)⁺ 426; ¹H NMR (CD₃OD) δ_{H} 7.96-7.98 (d, 1H), 7.78-7.80 (d, 1H), 7.73-7.75 (d, 1H), 7.62-7.66 (m, 2H), 7.22-7.33 (m, 4H), 7.14-7.17 (d, 2H), 6.99 (d,,1H), 6.89-6.92 (m, 3H), 4.69 (s, 2H).

### Example 60

5-[l-(4-Hydroxy-phenyl)-1H-benzoimidazol-2-yl]-thiophene-2-carboxylic acid methyl ester

Thiophene-2,5-dicarboxylic acid dimethyl ester

A mixture of concentrated sulfuric acid (150 μL) and of 2,5-thiophenedicarboxylic acid (1.0 g, 5.81 mmol) in of methanol (15 ml) were heated to reflux under nitrogen over night. The reaction mixture was cooled to room temperature and a white crystalline solid was filtered and dried (MgSO₄) to give thiophene-2,5-dicarboxylic acid dimethyl ester (0.995 g, 4.97 mmol, 86% yield). ¹H NMR (CD₃OD) δ_{H} 7.73 (s, 2H), 3.87 (s, 6H).

Thiophene-2,5-dicarboxylic acid monomethyl ester

To a gently refluxing solution of thiophene-2,5-dicarboxylic acid dimethyl ester (0.430 g, 2.15 mmol) in of dioxane/methanol (1:2, 1.5 ml) was added an aqueous solution of sodium hydroxide (0.086 g, 2.15 mmol in 0.5 ml water). The reaction mixture was stirred for 5 h and then cooled to room temperature. After adjusting the solution to pH 2 with 1N hydrochloric acid, the reaction mixture was extracted with ethyl acetate (3×30 ml) which was then washed with brine (1×5 ml), dried (MgSO₄) and concentrated by vacuum to give thiophene-2,5-dicarboxylic acid monomethyl ester as a white solid (0.342 g, 86% yield). ¹H NMR (CD₃OD) δ_{H} 7.72-7.73 (d, 1H), 7.69-7.70 (d, 1H), 3.87 (s, 3H).

5-[2-(4-Methoxy-phenylamino)-phenylcarbamoyl]-thiophene-2-carboxylic acid methyl ester

To a solution of N-(4-methoxy-phenyl)-benzene-1,2-diamine bis hydrochloride (0.416 g, 1.45 mmol), thiophene-2,5-dicarboxylic acid monomethyl ester, (0.323 g, 1.74 mmol), triethylamine (1.7 ml, 11.6 mmol) and a catalytic amount of 4-dimethylaminopyridine in methylene chloride (10 ml), was added of 1-propanephosphonic acid cyclic anhydride (1.3 ml, 2.18 mmol) as 50% solution in ethyl acetate. The mixture was stirred under nitrogen over night at room temperature. The reaction mixture was diluted with ethyl acetate (30 ml) and washed first with saturated sodium bicarbonate (2 ×10 ml) and then with brine (1 ×10 ml). The organic phase was dried over magnesium sulfate and concentrated. The solid orange product was purified by refluxing in methanol followed by a hot filtration. Evaporation of the filtrate gave 5-[2-(4-methoxy-phenylamino)-phenylcarbamoyl]-thiophene-2-carboxylic acid methyl ester (0.309 g, 0.809 mmol). MS (MH)+383; ¹H NMR (CDCl₃) δ_{H} 8.26 (s, 1H), 8.08 (d, 1H), 7.66-7.67 (d, 1H), 7.22-7.23 (d, 1H), 7.13-7.15 (m, 3H), 6.77-6.82 (m, 4H), 5.36 (s, 1H), 3.89 (s, 3H), 3.75 (s, 3H).

5-[1-(4-Methoxy-phenyl)-1H-benzoimidazol-2-yl]-thiophene-2-carboxylic acid methyl ester

A mixture of 5-[2-(4-methoxy-phenylamino)-phenylcarbamoyl]-thiophene-2-carboxylic acid methyl ester (0.280 g, 0.732 mmol and glacial acetic acid (15 ml) was heated at 80° C. under an atmosphere of N₂. Heptane (50 ml) was added as an azeotrope and the acetic acid was concentrated by vacuum. Two more aliquots of heptane were added and the remaining acetic acid was evaporated, to give 5-[1-(4-methoxy-phenyl)-1H-benzoimidazol-2-yl]-thiophene-2-carboxylic acid methyl ester leaving (0.209 g, 0.574 mmol, 75% yield) as a white solid. MS (MH)⁺ 365; ¹H NMR (CDCl₃) δ_{H} 7.83-7.85 (d, 1H), 7.58-7.59 (d, 1H), 7.30-7.34 (m, 3H), 7.23-7.27 (m, 1H), 7.05-7.11 (m, 3H), 6.98-6.99 (d, 1H), 3.93 (s, 3H), 3.86 (s, 3H).

5-[l-(4-Hydroxy-phenyl)-1H-benzoimidazol-2-yl]-thiophene-2-carboxylic acid methyl ester

To a solution of 5-[1-(4-methoxy-phenyl)-1H-benzoimidazol-2-yl]-thiophene-2-carboxylic acid methyl ester (0.039 g, 0.107 mmol) in methylene chloride (1.5 ml) cooled to −78° C. under an atmosphere of nitrogen, was added) of boron tribromide (0.43 ml, 0.43 mmol) as a 1.0M solution in methylene chloride. The mixture was allowed to stir over night slowly warming to room temperature. Methanol (5 ml) was added to the reaction mixture and stirring continued for 3 h. The solution was neutralized (pH=7) with saturated sodium bicarbonate and was diluted with ethyl acetate (20 ml). The layers were separated and the aqueous layer was further extracted with EtOAc (2×10 ml). (MgSO₄) and concentrate by vacuum to give 5-[1-(4-hydroxy-phenyl)-1H-benzoimidazol-2-yl]-thiophene-2-carboxylic acid methyl ester (0.015 g, 0.427 mmol). MS (MH)⁺ 351; ¹H NMR (d₆-dmso) δ_{H} 10.13 (s, 1H), 7.71-7.73 (d, 1H), 7.65-7.66 (d, 1H), 7.32-7.34 (d, 2H), 7.21-7.29 (m, 2H), 6.92-7.02 (m, 4H), 3.77 (s, 3H).

### Example 61

5-[1-(4-Hydroxy-phenyl)-1H-benzoimidazol-2-yl]-thiophene-2-carboxylic acid benzhydryl-amide

5-[1-(4-Methoxy-phenyl)-1H-benzoimidazol-2-yl]-thiophene-2-carboxylic acid

To a solution of 5-[1-(4-Hydroxy-phenyl)-1H-benzoimidazol-2-yl]-thiophene-2-carboxylic acid methyl ester (0.777 g, 2.13 mmol) in THF/methanol (1:1, 10 ml) was added 5N sodium hydroxide (4.3 ml, 21.3 mmol). The reaction material was warmed until homogenous and then stirred at room temperature over night. The solution was acidified to pH 2 with 1N hydrochloric acid; and the light yellow solid was filtered and dried by vacuum to give 5-[1-(4-methoxy-phenyl)-1H-benzoimidazol-2-yl]-thiophene-2-carboxylic acid (0.672 g, 1.92 mmol). MS (MH)⁺ 351; ¹H NMR (dmso) δ_{H} 13.33 (s, 1H), 7.74-7.76 (d, 1H), 7.57-7.58 (d, 1H), 4.48-7.50 (d, 2H), 7.22-7.30 (m, 2H), 7.18-7.20 (d, 2H), 7.00-7.02 (d, 1H), 6.88-6.89 (d, 1H), 3.86 (s, 3H).

5-[1-(4-Hydroxy-phenyl)-1H-benzoimidazol-2-yl]-thiophene-2-carboxylic acid benzhydryl-amide

To a solution of 5-[l-(4-methoxy-phenyl)-1H-benzoimidazol-2-yl]-thiophene-2-carboxylic acid (0.043 g, 0.123 mmol), (0.019 g, 0.102 mmol), triethylamine (0.235 ml, 1.68 mmol), aminodiphenyl methane (0.019 g, 0.102 mmol and 4-dimethylaminopyridine (catalytic amount) in methylene chloride (2 ml) was added 50% 1-propanephosphonic acid cyclic anhydride in ethyl acetate (0.150 ml, 0.252 mmol). The solution was stirred for 48 h at room temperature and under nitrogen. Saturated sodium bicarbonate (3 ml) was added and the reaction mixture was extracted with ethyl acetate. The combined ethyl acetate extracts were washed with brine, dried over sodium sulfate and concentrated. The crude 5-[1-(4-methoxy-phenyl)-1H-benzoimidazol-2-yl]-thiophene-2-carboxylic acid benzhydryl-amide was used in the next step without further purification. MS (MH)⁺ 516; ¹H NMR (CDCl₃) δ_{H} 7.79-7.81 (d, 1H), 7.20-7.37 (m, 14H), 7.03-7.08 (m, 3H), 6.94-6.95 (d, 1H), 6.44-6.46 (d,1H), 6.31-6.33 (d,1H), 3.89 (s, 3H).

5-[1-(4-Hydroxy-phenyl)-1H-benzoimidazol-2-yl]-thiophene-2-carboxylic acid benzhydryl-amide

To a solution of 5-[1-(4-Methoxy-phenyl)-1H-benzoimidazol-2-yl]-thiophene-2-carboxylic acid benzhydryl-amide (0.022 g, 0.043 mmol) in CH₂Cl₂ (1 ml) cooled to −78° C. under an atmosphere of N₂ was added BBr₃ as a 1.0M solution in CH₂Cl₂ (0.14 ml, 0.14 mmol). The solution was stirred O/N slowly warming to RT. The reaction was quenched upon the addition of MeOH (0.5 ml). The reaction mixture was neutralized with NaHCO₃ solution and diluted with water (15 ml). The mixture was extracted with EtOAc (3×15 ml). The combined extracts were dried (MgSO₄), filtered and concentrated by vacuum. The residue was purified by preparatory TLC (SiO₂, eluting with 10% methanol in methylene chloride), to give 5-[l-(4-hydroxy-phenyl)-1H-benzoimidazol-2-yl]-thiophene-2-carboxylic acid benzhydryl-amide (0.016 g, 31.9 μmol). MS (MH)⁺ 502; ¹H-NMR (CD₃OD) δ_{H} 7.67-7.69 (d, 1H), 7.65-7.66 (d, 1H), 7.21-7.32 (m, 14H), 7.09-7.10 (d, 1H), 7.05-7.07 (d, 1H), 6.98-6.01 (d, 2H), 6.34 (s,1H).

### Example 62

4-(4-Phenyl-5-trifluoromethyl-isoxazol-3-yl)-phenol

1-(4-Methoxy-phenyl)-2-phenyl-ethanone oxime

To a solution of deoxy-4-methoxybenzoin (2.00 g, 8.88 mmol) in EtOH (50 ml) and pyridine (30 ml) was added hydroxylamine hydrochloride (3.08 g, 44.4 mmol). The reaction was stirred at room temperature overnight. The reaction mixture was concentrated in vacuo and diluted with Et₂O (50 ml). The mixture was washed with 10% HCl (2×50 ml) and brine (1×50 ml). The organic layer was dried (Na₂SO₄), filtered and concentrated in vacuo to give 1-(4-methoxy-phenyl)-2-phenyl-ethanone oxime (1.4 g, 5.82 mmol).

3-(4-Methoxy-phenyl)-4-phenyl-5-trifluoromethyl4, 5,-dihydro-isoxazol-5-ol

To a solution of 1-(4-methoxy-phenyl)-2-phenyl-ethanone oxime (1.4 g, 5.82 mmol) in THF (30 ml) cooled to 0 ° C. was added n-BuLi (n-butyl lithium 4.65 ml, 0.012 mol) as a 2.5M solution in hexanes in a dropwise manner. Ethyl trifluoroacetate (1.24 g, 8.72 mmol) was then added to the reaction mixture. The reaction mixture was allowed to stir overnight. The reaction mixture was carefully quenched with acetic acid (3.0 g). The mixture was concentrated in vacuo and after washing the residual solid with toluene 3-(4-methoxy-phenyl)4-phenyl-5-trifluoromethyl-4,5,-dihydro-isoxazol-5-ol was obtained (1.84 g, 5.47 mmol).

3-(4-Methoxy-phenyl)-4-phenyl-5-trifluoromethyl-isoxazole

A mixture of 3-(4-Methoxy-phenyl)4-phenyl-5-trifluoromethyl4,5,-dihydro-isoxazol-5-ol (0.75 g, 2.22 mmol) and p-toluene sulphonic acid (0.08 g, 0.443 mmol) was heated in toluene (300 ml) at reflux with a Dean-Stark trap for 5 hours. The reaction mixture was washed with sat. NaHCO₃ (1×50 ml) and brine (1×50 ml), dried (Na₂SO₄) and concentrated in vacuo. The residue was purified by flash chromatography (SiO₂ 1:20 EtOAC:petroleum ether) to give 3-(4-methoxy-phenyl)-4-phenyl-5-trifluoromethyl-isoxazole (0.297 g, 0.957 mmol).

4-(4-Phenyl-5-trifluoromethyl-isoxazol-3-yl)-phenol

To a solution of 3-(4-methoxy-phenyl)-4-phenyl-5-trifluoromethyl-isoxazole (0.297 g, 0.957 mmol) in CH₂Cl₂ (2.8 ml) cooled to 0° C. was added BBr₃ as a 1.0M solution in CH₂Cl₂ (2.8 ml, 2.8 mmol). The reaction mixture was slowly allowed to warm to room temperature and stirring was continued overnight. MeOH (2 ml) was carefully added to the reaction mixture and stirring was continued for a further two hours at room temperature. The mixture was concentrated in vacuo and the residue purified by flash chromatography (SiO₂, 20% to 50% EtOAc/petroleum ether) to give 4-(4-Phenyl-5-trifluoromethyl-isoxazol-3-yl)-phenol (0.251 g, 0.822 mmol). MS 306 (M+1).

1. A compound of formula (I) 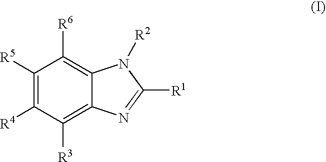
or the pharmaceutically acceptable salts thereof; wherein:
R¹ and R² are each independently selected from the group consisting of (C₁-C₆)alkyl; phenyl; (C₂-C₆)heteroaryl; (C₃-C₈)cycloalkyl, and (C₄-C₈)cycloalkenyl;
wherein the (C₁-C₆)alkyl; phenyl; (C₂-C₆)heteroaryl; (C₃-C₈)cycloalkyl; or (C₄-C₈)cycloalkenyl groups of R¹ or R² are optionally substituted by from 1 to 3 substituents independently selected from the group consisting of:
halogen; (C₁-C₆)alkyl; (C₃-C₈)cycloalkyl; (C₄-C₈)cycloalkenyl; (C₁-C₆)alkoxy; hydroxy; R¹² CO₂, R¹²R¹³NCO, R¹²R¹³N; (C₁-C₆)alkylcarbonyl, —CHO, cyano, thio; (C₁-C₆)alkylthio; (C₁-C₆)alkylsulfonyl; (C₁-C₆)alkylsulfinyl; hydroxy(C₁-C₆)alkyl; (C₁-C₆)alkoxycarbonylamino; (C₁-C₆)alkylcarbonylamino; (C₁-C₆)alkenylcarbonylamino; (C₁-C₆)alkoxycarbonyloxy; R¹² R¹³ N(C₁-C₆); R¹²R¹³N(C₁-C₆)alkoxy; R^{12l R}¹³N(C₁-C₆alkyl)S; N-morpholino(CH₂)ₙO; or —R¹²R¹³N(CH₂)ₙS(O)ₓ; wherein the (C₁-C₆)alkyl; (C₃-C₈)cycloalkyl; (C₄-C₈)cycloalkenyl; (C₁-C₆)alkoxy; (C₁-C₆)alkylcarbonyl; (C₁-C₆)alkylthio; (C₁-C₆)alkylsulfonyl; (C₁-C₆)alkylsulfinyl; (C₁-C₆)alkoxycarbonylamino; (C₁-C₆)alkylcarbonylamino; (C₁-C₆)alkenylcarbonylamino; or (C₁-C₆)alkoxycarbonyloxy groups are each optionally further substituted by from 1 to 3 substituents independently selected from the group consisting of:
halogen, (C₁-C₆)alkyl; (C₃-C₈)cycloalkyl; (C₄-C₈)cycloalkenyl; (C₁-C₆)alkoxy, hydroxy, R¹² CO₂, R¹²R¹³NCO, R¹²R¹³N;(C₁-C₆)alkylcarbonyl, —CHO, cyano, thio; R¹² SO₂(C₁-C₆)alkyl; R¹² CO₂(C₁-C₆)alkyl; R¹²R¹³NCO(C₁-C₆)alkyl; R¹² CO(C₁-C₆)alkyl; R¹² SO₂(C₁-C₆)alkoxy; R¹² CO₂(C₁-C₆)alkoxy; R¹²R¹³NCO(C₁-C₆)alkoxy; R¹²CO(C₁-C₆)alkoxy; R¹²R¹³ N SO₂(C₁-C₆)alkyl; and R¹²R¹³N SO₂(C₁-C₆) alkoxy; wherein:
R¹² and R¹³ are each independently selected from the group consisting of hydrogen; (C₁-C₇)alkyl; (C₃-C₈)cycloalkyl; (C₄-C₈)cycloalkenyl;(C₆-C₁₀) aryl; (C₂-C₁₀)alkenyl, (C₂-C₁₀)alkynyl; (C₂-C₄)heteroaryl; (C₁-C₆)alkylaryl; (C₁-C₆) alkyl(C₂-C₆)heteroaryl; (C₂-C₆)alkoxyaryl; (C₂-C₆) alkoxy(C₂-C₆)heteroaryl; or R¹² and R¹³ taken together form a three to eight membered heterocyclic ring having 1 to 3 heteroatoms; n is from 0 to 5; and x is 1 or 2;
or R¹ and R² are each independently a group of the formula: 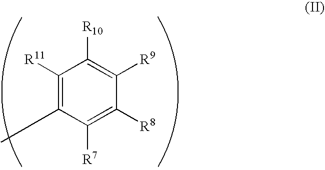
wherein R⁷, R⁸ R¹⁰ and R¹¹ are each independently hydrogen; hydroxy; (C₁-C₆) alkyl; (C₁-C₆)alkoxy; or halogen;
R⁹ is hydroxy; (C₁-C₆) alkoxy; (C₁-C₆)alkoxycarbonyloxy; (C₁-C₆)alkylcarbonyloxy; (C₃-C₈)cycloalkoxy; (C₄-C₈)cycloalkenyloxy; or (C₆-C₁₂) aryloxy; and
R³, R⁴, R⁵ and R⁶ are each independently hydrogen, hydroxy; (C₁-C₆)alkyl; (C₁-C₆)alkoxy; or halogen;
with the proviso that at least one of R¹ or R² must be the group of formula (II)

2. A compound according to claim 1, wherein R¹ is phenyl or (C₂-C₆) heteroaryl.

3. A compound according to claim 2, wherein the (C₂-C₆) heteroaryl is thienyl; furyl;pyrrolyl; isoxazolyl; isothiazolyl or thiodiazolyl.

4. A compound according to claim 1, wherein R¹ is phenyl optionally substituted by R¹²CO₂ or R¹²R¹³NC(O).

5. A compound according to claim 1, wherein R² is a group of formula (II).

6. A compound according to claim 5,wherein R⁷, R⁸ R¹⁰ and R¹¹ are hydrogen and R⁹ is hydroxy or (C₁-C₆)alkoxy.

7. A compound according to claim 1, wherein R³, R⁴, R⁵ and R⁶ are hydrogen.

8. A compound according to claim 1, wherein R¹ is phenyl or (C₂-C₆)heteroaryl; R² is a group of formula (II); and R³, R⁴, R⁵ and R⁶ are hydrogen.

9. A compound according to claim 8, wherein (C₂-C₆)heteroaryl is thienyl; furyl; pyrrolyl; isoxazolyl; isothiazoyl or thiodiazolyl; R⁷, R⁸, R¹⁰ and R¹¹ are hydrogen; and R⁹ is hydroxy or (C₁-C₆)alkoxy.

10. A compound according to claim 1, wherein R¹ is phenyl optionally substituted by R¹²CO₂ or R¹²R¹³NC(O); R² is a group of formula (II); and R³, R⁴, R⁵ and R⁶ are hydrogen.

11. A compound according to claim 1, wherein the compound of Formula (I) is selected from the group consisting of:
(±)4-(2-sec-butyl-benzoimidazol-1-yl)-phenol;
4-(2-cyclopropyl-benzoimidazol-1-yl)-phenol 4-[2-(4-iodo-phenyl)-benzoimidazol-1-yl]-phenol;
4-(2-thiophen-3-yl-benzoimidazol-1-yl-phenol; 4-(2-thiophen-2-yl-benzoimidazol-1-yl)phenol;
4-[2-(1-methyl-1H-pyrrol-2-yl)-benzoimidazol-1-yl]-phenol;
4-[2-(3,5-dimethyl-isoxazol-4-yl)-benzoimidazol-1-yl)-phenol;
4-[2-(3-bromo-thiophen-2-yl)-benzoimidazol-1-yl]-phenol;
4-(2-isothiazol-4-yl-benzoimidazol-1-yl)-phenol;
4-[2-(4-methyl-isothiazol-5-yl)-benzoimidazol-1-yl]-phenol;
4-[2-(4-methyl-[1,2,3]thiadiazol-5-yl)-benzoimidazol-1-yl]-phenol;
4-[2-(3-chloro-thiophen-2-yl)-benzoimidazol-1-yl]-phenol;
4-[2-(1-ethyl-1H-pyrrol-2-yl)-benzoimidazol-1-yl]-phenol;
4-(2-furan-3-yl-benzoimidazol-1-yl-phenol;
4-[2-(3-methyl-furan-2-yl)-benzoimidazol-1-yl]-phenol;
4-(2-furan-2-yl-benzoimidazol-1-yl)-phenol;
4-[2-(3-ethyl-isoxazol4-yl)-benzoimidazol-1-yl-phenol;
4-[2-(3-cyclopropyl-isoxazol-4-yl)-benzoimidazol-1-yl]-phenol;
4-[2-(3-ethyl-5-methyl-isoxazol-4-yl)-benzoimidazol-1-yl]-phenol;
4-[2-(5-methyl-isoxazol4-yl)-benzoimidazol-1-yl]-phenol;
4-[2-(3-methyl-isoxazol-4-yl]-phenol;
4-[2-(2-methyl-thiophen-3-yl)-benzoimidazol-1-yl]-phenol;
4-[2-(2-methyl-furan-3-yl)-benzoimidazol-1-yl]-phenol;
4-[2-(2,5-dimethyl-furan-3-yl)benzoimidazol-1-yl]-phenol;
4-[2-(2,5-dimethyl-furan-3-yl)benzoimidazol-1-yl]-phenol;
4-[2-(1-propyl-1H-pyrrol-2-yl)-benzoimidzol-1-yl]-phenol;
4-[2-(1-isopropyl-1H-pyrrol-2-yl)-benzoimidazol-1-yI]-phenol;
3-methyl-4-[2-( 1-methyl-1H-pyrrol-2-yl)-benzoimidazol-1-yl]-phenol;
4-[2-(3,5-dimethyl-isoxazol4-yl)-benzoimidazol-1-yl]-3-methyl-phenol;
4-[2-(3-methyl-thiophen-2-yl)-benzoimidazol-1-yl]-phenol;
4-(2-isothiazol-5-yl-benzoimidazol-1-yl)-phenol;
4-[1-(4-Hydroxy-phenyl)-1H-benzoimidazol-2-yl]-benzoic acid methyl ester;
4-[l-(4-Hydroxyl phenyl)-l H-benzoimidazol72-yl]-benzoic acid ethyl ester;
4-[1-(4-Hydroxy-phenyl)-1H-benzoimidazol-2-yl]-benzoic acid isopropyl ester;
4-[1-(47Hydroxy-phenyl)-1H-benzoimidazol-2-yl]-N-isopropyl-benzamide;
4-[1-(4-Hydroxy-phenyl)-1H-benzoimidazol-2-yl]-N-(1-phenyl-ethyl)-benzamide;
4-[1-(4-Hydroxy-phenyl)-1H-benzbimidazol-2-yl]-N-(1-pheny-ethyl)-benzamide; and
4-[1-(4-Hydroxy-phenyl)-1H-benzoimidazol-2-yl]-N-thiophen-2-ylmethyl-benzamide, or the pharmaceutically accepted salts thereof.

12. A pharmaceutical composition for antagonizing or agonizing an estrogen receptor in a mammal comprising an estrogen receptor antagonizing or agonizing effective amount of a compound of formula (I) according to claim 1, or a pharmaceutically accepted salt thereof, and a pharmaceutically acceptable carrier.

13. A pharmaceutical composition for selectively antagonizing or agonizing an ERβ estrogen receptor in a mammal comprising an ERβ estrogen receptor antagonizing or agonizing effective amount of a compound of formula (I) as defined in claim 1, or the pharmaceutically acceptable salts thereof and a pharmaceutically acceptable carrier.

14. A pharmaceutical composition comprising an agent selected from the group consisting of an anabolic agent; a growth hormone; a growth hormone secretagogue; a prostaglandin agonist/antagonist; a parathyroid hormone; sodium fluoride; and a mixture thereof; the pharmaceutical composition further comprising a compound of formula (I) according to claim 1.

15. A method of treating a condition which presents with low bone mass in a mammal comprising administering to the mammal a compound of formula (I) according to claim 1, a prodrug thereof or a pharmaceutically acceptable salt, or a stereoisomeric mixture of said compound, salt or prodrug.

16. The method of claim 15 wherein the condition is osteoporosis.

17. A kit comprising: a) an amount of a compound of Formula (I) as defined in claim 1; b) an amount of a second compound comprising an anabolic agent; a growth hormone; a growth hormone secretagogue; a prostaglandin agonist/antagonist; a parathyroid hormone; sodium fluoride; or a mixture thereof; and c) a container.

18. A method of treating a disease mediated by the estrogen receptor in a mammal, comprising administering to the mammal a therapeutically effective amount of a compound of formula (I) according to claim 1 in a pharmaceutically effective carrier.

19. The method of claim 18 wherein the disease is selected from the group consisting of perimenopausal or postmenopausal syndrome, osteoporosis, atrophy of skin or vagina, elevated serum cholesterol levels, cardiovascular disease, Alzheimer's disease, estrogen dependent cancers, including breast or uterine cancer, a prostatic disease, benign prostatic hyperplasia, prostate cancer, obesity, endometriosis, bone loss, uterine fibrosis, aortal smooth muscle cell proliferation, acne, hirsutism, dysfunctional uterine bleeding, dysmenorrehea, male infertility, MED, psychological and behavioral symptoms during menstruation, ulcerative mucositis, uterine fibroid disease, restenosis, atherosclerosis, musculoaponeurotic fibromatosis, alopecia, autoimmune disease, cartilage degeneration, delayed puberty, demyelinating disease, dysmyelinating disease, hypoglycemia, lupus erythematosus, myocardial infection, ischemia, thromboembolic disorder, obsessive compulsive disorder, ovarian dysgenesis, post menopausal central nervous system (CNS) disorder, pulmonary hypertension, reperfusion damage, resistant neoplasm, rheumatoid arthritis, seborrhea, sexual precocity, thyroiditis, Turner's syndrome, and hyperlipidemia and female sexual dysfunction.

20. A method for selectively antagonizing or agonizing the ERβ estrogen receptor in a mammal comprising an ERβ estrogen receptor antagonizing or agonizing effective amount of a compound of formula (I) according to claim 1.

21. A pharmaceutical composition for antagonizing or agonizing the estrogen receptor in a mammal comprising an estrogen receptor antagonizing or agonizing effective amount of a compound selected from the group consisting of: 4-(5-phenyl-2-trifluoromethyl-3H-imidazol-4-yl)-phenol; 4-[5-[(4-hydroxy-phenyl)2-trifluoromethyl-3-H-imidazol4-yl]-phenol; 4-[5-[(4-methoxy-phenyl)-2-trifluoromethyl-1H-imidazol-4-yl]-phenol; and 4-(4-phenyl-5-trifluoromethyl-isoxazol-3-yl)-phenol, or a pharmaceutically accepted salt thereof, and a pharmaceutically acceptable carrier.
